SEQUENCE LISTING (48 sequences)

SEQ ID NO: 1 - DNA, 20 bp, Artificial Sequence
  synthetic oligonucleotide primer
atgcaaggattcaccgaaag

SEQ ID NO: 2 - DNA, 20 bp, Artificial Sequence
  synthetic oligonucleotide primer
atagcggcctttaccaacct

SEQ ID NO: 3 - DNA, 20 bp, Artificial Sequence
  synthetic oligonucleotide primer
gtcgtgacactcccattcct

SEQ ID NO: 4 - DNA, 20 bp, Artificial Sequence
  synthetic oligonucleotide primer
atagcggccttttccaatct

SEQ ID NO: 5 - DNA, 20 bp, Artificial Sequence
  synthetic oligonucleotide primer
atggaacataccgcttttgc

SEQ ID NO: 6 - DNA, 20 bp, Artificial Sequence
  synthetic oligonucleotide primer
gtgagcctctcgtctccaac

SEQ ID NO: 7 - DNA, 20 bp, Artificial Sequence
  synthetic oligonucleotide primer
gtttcccacatcaagctggt

SEQ ID NO: 8 - DNA, 20 bp, Artificial Sequence
  synthetic oligonucleotide primer
gtttcccacatcaagctggt

SEQ ID NO: 9 - DNA, 20 bp, Artificial Sequence
  synthetic oligonucleotide primer
acacagcccacttcaaatcc

SEQ ID NO: 10 - DNA, 20 bp, Artificial Sequence
  synthetic oligonucleotide primer
ggtgcctcttttctctgcac

SEQ ID NO: 11 - DNA, 20 bp, Artificial Sequence
  synthetic oligonucleotide primer
catcatcacgtggaacgaac

SEQ ID NO: 12 - DNA, 20 bp, Artificial Sequence
  synthetic oligonucleotide primer
cttgtggacaaccacctcct

SEQ ID NO: 13 - DNA, 20 bp, Artificial Sequence
  synthetic oligonucleotide primer
aactgcttgcagtgggaagt

SEQ ID NO: 14 - DNA, 20 bp, Artificial Sequence
  primer
ggaaacttccggttcctctc

SEQ ID NO: 15 - DNA, 20 bp, Artificial Sequence
  primer
tctggtctgcctctcttggt

SEQ ID NO: 16 - DNA, 20 bp, Artificial Sequence
  primer
tctcgtgtctcagcatcacc

SEQ ID NO: 17 - DNA, 20 bp, Artificial Sequence
  primer
caaggtttacgggctcatgt

SEQ ID NO: 18 - DNA, 20 bp, Artificial Sequence
  primer
gagaaagccagcgatgtagg

SEQ ID NO: 19 - DNA, 20 bp, Artificial Sequence
  primer
ttagcggcctgagagatgtt

SEQ ID NO: 20 - DNA, 20 bp, Artificial Sequence
  primer
caggagagacggttgagagc

SEQ ID NO: 21 - DNA, 20 bp, Artificial Sequence
  primer
tgggttggaagcaaaaagac

SEQ ID NO: 22 - DNA, 20 bp, Artificial Sequence
  primer
cctgctttgcttctttggtc

SEQ ID NO: 23 - DNA, 20 bp, Artificial Sequence
  primer
ctgcagcatgcctaatgaga

SEQ ID NO: 24 - DNA, 20 bp, Artificial Sequence
  primer
cgtctgcctagatgcaatca

SEQ ID NO: 25 - DNA, 20 bp, Artificial Sequence
  primer
tgtgttctgggcagtttctg

SEQ ID NO: 26 - DNA, 20 bp, Artificial Sequence
  primer
ggcagtacagggctgagaag

SEQ ID NO: 27 - DNA, 20 bp, Artificial Sequence
  primer
ggcgaaggcattacagtgtt

SEQ ID NO: 28 - DNA, 20 bp, Artificial Sequence
  primer
tgcacatacaaatggcctgt

SEQ ID NO: 29 - DNA, 20 bp, Artificial Sequence
  primer
gcaagttttgcgatgtgaga

SEQ ID NO: 30 - DNA, 20 bp, Artificial Sequence
  primer
ggcatcttccagagtgaagc

SEQ ID NO: 31 - DNA, 20 bp, Artificial Sequence
  primer
caggtgcttgtgtggagaga

SEQ ID NO: 32 - DNA, 20 bp, Artificial Sequence
  primer
catgttggtgtccaggtcag

SEQ ID NO: 33 - DNA, 20 bp, Artificial Sequence
  primer
ggtgtcaaagaagcctctgc

SEQ ID NO: 34 - DNA, 20 bp, Artificial Sequence
  primer
acctcaatgcgatttgttcc

SEQ ID NO: 35 - DNA, 20 bp, Artificial Sequence
  primer
cactccacagcaggaagtga

SEQ ID NO: 36 - DNA, 20 bp, Artificial Sequence
  primer
ctcagtgaaagctccggttc

SEQ ID NO: 37 - DNA, 20 bp, Artificial Sequence
  primer
tggaagtggcccatttagag

SEQ ID NO: 38 - DNA, 20 bp, Artificial Sequence
  primer
tgacgcttttctcgtttgtg

SEQ ID NO: 39 - DNA, 20 bp, Artificial Sequence
  primer
tgctgtggctctatgacagg

SEQ ID NO: 40 - DNA, 20 bp, Artificial Sequence
  primer
accccttcgtttgaggagtt

SEQ ID NO: 41 - DNA, 20 bp, Artificial Sequence
  primer
tgagcctttccagcaagttt

SEQ ID NO: 42 - DNA, 20 bp, Artificial Sequence
  primer
cttcccggtctcaggtatca

SEQ ID NO: 43 - DNA, 20 bp, Artificial Sequence
  primer
ttcaaggcaagcgaggtact

SEQ ID NO: 44 - DNA, 20 bp, Artificial Sequence
  primer
aatgcagcatacccttctgg

SEQ ID NO: 45 - DNA, 20 bp, Artificial Sequence
  primer
ttcttcaaggtgagcgaggt

SEQ ID NO: 46 - DNA, 20 bp, Artificial Sequence
  primer
tagttggcagcgtagccttt

SEQ ID NO: 47 - DNA, 20 bp, Artificial Sequence
  primer
gaaaacagcagcagtgacca

SEQ ID NO: 48 - DNA, 20 bp, Artificial Sequence
  primer
ggtggcgttcatgtaggagt

(19) United States
(12) Patent Application Publication
(10) Pub. No.: US 2015/0184247 A1
(43) Pub. Date: Jul. 2, 2015

(54) GENE EXPRESSION PROFILES ASSOCIATED WITH METASTATIC BREAST CANCER

(71) Applicant: MEMORIAL SLOAN-KETTERING CANCER CENTER, New York, NY (US)

(72) Inventor: Filippo G. Giancotti, New York, NY (US)

(73) Assignee: MEMORIAL SLOAN-KETTERING CANCER CENTER, New York, NY (US)

(21) Appl. No.: 14/395,759

(22) PCT Filed: Apr. 22, 2013
(86) PCT No.: PCT/US13/37609

§ 371 Date: Oct. 20, 2014

Related U.S. Application Data
(60) Provisional application No. 61/636,215, filed on Apr. 20, 2012.
(60) Provisional application No. 61/668,805, filed on Jul. 6, 2012.

(51) Int. Cl. C12Q 1/68

(52) U.S. Cl. CPC C12Q 1/6886; C12Q 2600/158

(57) ABSTRACT

A gain-of-function retroviral cDNA screen reveals that Coco, a secreted antagonist of TGF-β ligands, induces solitary mammary carcinoma cells, which have disseminated to others sites and undergone an extended period of dormancy, to exit from dormancy at lung metastatic sites. Evidence indicates that Coco awakens tumor progenitor cells that have extravasated into the lung by inhibiting stroma-derived Bone Morphogenetic Proteins (BMP). Whereas Coco enhances the manifestation of traits associated with cancer stem cells, activation of canonical BMP signaling suppresses these processes. Expression of Coco correlates with lung metastatic capacity in a number of human breast cancer cell lines and induces a gene expression signature that predicts metastatic relapse to the lung but not to the bone in advanced primary tumors. Disclosed herein is the gain-of-function retroviral cDNA screen and a gene signature predictive of selective relapse and gene expression microarrays or panels based thereon for use in prognostication of relapse.

### CROSS-REFERENCE TO RELATED APPLICATIONS

This application claims the priority of U.S. provisional application Ser. No. 61/636,215 filed Apr. 20, 2012 and Ser. No. 61/668,805 filed Jul. 6, 2012. The contents of each is incorporated herein by reference in its entirety.

### STATEMENT REGARDING FEDERALLY SPONSORED RESEARCH OR DEVELOPMENT

This invention was made under grant number NIH grant P01 CA094060 from the National Institutes of Health. The government has certain rights in the invention.

### FIELD OF THE INVENTION

The present invention relates generally to gene expression profiles that are informative of metastasis and in particular, to gene expression panels and arrays as well as methods for predicting metastasis of cancer cells and overall survival.

### BACKGROUND OF THE INVENTION

The process through which cancer cells acquire metastatic capacity is complex. Unrestrained proliferation, resistance to pro-apoptotic insults, and invasion through tissue boundaries are not sufficient for metastatic dissemination. In order to colonize distant organs, tumor cells must also adapt to the local microenvironment of the target organ (Fidler, 2003; Nguyen et al., 2009; Valastyan and Weinberg, 2011). The large majority of tumor cells that extravasate in the parenchyma of a distant organ undergo cell death or enter into proliferative quiescence, making adaptation one of the most inefficient steps of metastasis (Cameron et al., 2000; Luzzi et al., 1998; Wong et al., 2001). The mechanisms that endow metastatic tumor cells with the properties required for adaptation are poorly understood. Recent studies on breast cancer metastasis have emphasized the importance of initial survival signals (Chen et al., 2011) and adhesive and signalling interactions with components of the extracellular matrix (Malanchi et al., 2012; Oskarsson et al., 2011; Shibue and Weinberg, 2009).

Metastatic relapse usually occurs at a significant time following surgical resection of the primary tumor. In breast cancer, this lag time often ranges from 5 to 10 years but can be as long as 20 years (Karrison et al., 1999). Early dissemination followed by a period of tumor cell dormancy provides a plausible explanation for this prevalent clinical behavior (Aguirre-Ghiso, 2007; Weinberg 2008). In agreement with this model, disseminated cancer cells can be detected in the bone marrow of patients with early-stage breast cancer (Pantel et al., 2008). In addition, premalignant lesions occurring in mouse models as well as ductal carcinomas in situ in women release potentially metastatic cells into the circulation (Husemann et al., 2008; Podsypanina et al., 2008). These observations suggest that a fraction of tumor cells that have extravasated in the target organ remain dormant for extended periods as a consequence of their inability to exit from proliferative quiescence (solitary tumor cell dormancy) or that they give rise to micrometastatic lesions that are unable to outgrow until they avert immunosurveillance and elicit a supportive angiogenic response (micrometastatic dormancy) (Aguirre-Ghiso, 2007).

The cancer stem cell model postulates that tumor growth is fueled by a limited, although not necessarily small, number of dedicated cancer stem cells, which undergo self-renewal as well as generate rapidly dividing cells and aberrantly differentiated post-mitotic cells (Clevers, 2011; Gupta et al., 2009; Shackleton et al., 2009). It has been proposed that the cancer stem cells are endowed with metastatic capacity, whereas the remaining tumor cells lack the migratory and the self-renewal capability necessary to colonize distant organs (Brabletz et al., 2005). In agreement with this model, re-expression of the luminal cell fate determinant GATA3 induces tumor cell differentiation and blocks dissemination and metastasis in MMTV-PyMT mice (Kouros-Mehr et al., 2008). In addition, phenotypic analysis suggests that the metastatic capacity of human pancreatic and colorectal cancers is restricted to a subpopulation of tumor cells that include cancer stem cells (Hermann et al., 2007; Pang et al., 2010). Finally, the Epithelial to Mesenchymal Transition (EMT) program that facilitates tumor dissemination produces cells endowed with the capacity to self-renew, suggesting that these two cellular processes are intimately linked (Mani et al., 2008).

Recent studies have linked the behavior of cancer stem cells to metastatic colonization of breast cancer. For example, the Inhibitors of differentiation (Id) 1 and 3 transcription factors and the miR200 and miR335 families of microRNAs promote the post-dissemination phase of breast cancer metastasis at least in part by modulating cancer stem cell function (Gupta et al., 2007b; Korpal et al., 2011; Shimono et al., 2009; Tavazoie et al., 2008). Furthermore, the extracellular matrix components tenascin C and osteonectin nurse outgrowing micrometastases by enhancing signalling pathways, such as Wnt and Notch, which enhance stem cell behavior (Malanchi et al., 2012; Oskarsson et al., 2011). These studies suggest that the tumor cells that are fated to colonize distant organs, the metastasis-initiating cells, share functional properties with the cancer stem cells.

The ability of metastasis-initiating cells to enter into, and eventually exit from, proliferative quiescence suggests an additional commonality with adult tissue stem cells. However, the relationship between cancer stem cell behavior and dormancy at metastatic sites is poorly understood.

A marker-based approach to tumor identification and characterization promises improved diagnostic and prognostic reliability. Gene microarrays have been used to identify diagnostic and prognostic biomarkers and to decipher the molecular mechanisms behind the clinical outcome or phenotype in various types of cancers. Increasingly, gene signatures are being identified and exploited as tools for disease detection as well as for prognosis and prospective assessment of therapeutic success. Genetic profiling of cancers, including breast cancer, may provide a more effective approach to cancer management and/or treatment. Currently, there exists a need for improved prognostic methods so that appropriate courses of prophylaxis and/or therapy may be provided for cancer patients.

### SUMMARY OF THE INVENTION

The present invention provides gene and protein expression profiles and methods of using them to identify those patients who are likely to experience metastasis of cells from the original tumor to the lung, with subsequent exit of those cells from dormancy to establish a new tumor. The present invention allows a treatment provider to identify those patients who may require more aggressive treatment or a particular treatment to specifically target potentially metastatic tumor cells.

The choice of genes of the array or panel is based on the identification of a gene signature associated with breast cancer metastasis. It is based on the observation that a particular set of genes that are differentially expressed in certain breast cancer cells mediate extravasation, metastasis and ultimately, the exit of metastatic tumor cells from dormancy, primarily in the lung. Accordingly, the present invention relates to gene expression profiles useful in predicting metastasis of cancer, e.g. breast cancer, assessing prognosis and/or metastasis-free survival and guiding treatment.

In a related aspect, the present invention relates to a method for evaluating the expression of a set of genes, the expression of which in breast cancer cells of a patient correlates with the likelihood of metastasis and poor prognosis.

In a related aspect, the present invention relates to a method for predicting the clinical outcome in a subject diagnosed with breast cancer, comprising contacting a breast cancer derived sample from the subject that contains a gene expression product with a gene expression array or panel consisting essentially of nucleic acids that are capable of hybridizing with a gene expression product of from two to 14 genes to determine the expression level of one or more predictive genes, or of their expression products, in a biological sample obtained from cancer cells from said subject.

In another aspect, the present invention relates to a method for the monitoring of breast cancer patients to determine the likelihood of metastasis to the lung. The method involves obtaining cancer cells from the patient, for example, during biopsy or at the time of surgical resection to remove a tumor, determining the expression level in the tumor cell(s) of at least two genes, in some embodiments from 2-5 genes, in other embodiments from 5-8 genes, in still other embodiments from 8-11 genes, in other embodiments up to 14 genes (13 unique genes), and comparing the expression levels of those genes with a reference expression level in known metastatic breast cancer cells, wherein a correlation between expression levels in the biological sample from the patient and reference expression levels in the metastatic breast cancer cells indicates a high likelihood of relapse/metastasis in the patient.

In yet another aspect, the present invention is directed to a method for predicting the likelihood of metastasis of breast cancer to the lung, said method comprising: contacting a breast cancer cell or tissue-derived sample containing a gene product with a gene panel or array consisting essentially of nucleic acids capable of hybridizing to a one or more genes selected from the group consisting of: ALDH6A1, NDRG1, PTDSS1, SCD, MYO5C, KIAA1199, TFPI, PNMA2, TMEM45A, PLAT, HSD17B10, EVI2B, PLAT and JAK1 to determine the expression level of said gene(s) in a breast cancer cell from the patient; and comparing said expression level with a reference expression level of said genes in metastatic breast cancer cells, wherein a correlation between expression levels in the breast cancer cell from the patient and expression levels in the metastatic breast cancer cells indicates a high likelihood of cancer metastasis to the lung.

In another embodiment, the present invention is directed to a method for predicting the likelihood of metastasis of breast cancer to the lung, said method comprising contacting a breast cancer cell or tissue-derived sample containing a gene product with a gene panel or array consisting essentially of nucleic acids capable of hybridizing to genes KIAA1199 and NDRG1 to determine the expression level of said genes in a breast cancer cell from the patient, and comparing said expression level with a reference expression level of said genes in metastatic breast cancer cells, wherein a correlation between expression levels in the biological sample from the patient and expression levels in the metastatic breast cancer cells indicates a high likelihood of cancer relapse.

In another embodiment, the present invention is directed to a method for predicting the likelihood of relapse in a breast cancer patient, said method comprising: determining the level of a Coco gene product (e.g. Coco protein or Coco mRNA) in a tissue or cell sample from a breast tumor of a patient, and comparing the level in the patient sample with a reference level of Coco gene product in normal individuals, wherein increased expression of Coco is positively correlated with increased likelihood of breast cancer relapse in the patient. Methods for determining the level of Coco expression in a cell or tissue are known in the art and include, for example, Q-PCR, microarray, immunoassay such as ELISA, Western blot and the like.

In a related aspect, the present invention relates to an expression panel or array comprising nucleic acids capable of hybridizing to an expression product of each of genes KIAA1199 and NDRG1. In one embodiment, the gene expression panel or array further comprises at least one additional nucleic acid that is capable of hybridizing to an expression product of an additional gene selected from the group consisting of ALDH6A1, PTDSS1, SCD, MYO5C, TFPI, PNMA2, TMEM45A, PLAT, HSD17B10, PLAT, EVI2B, and JAK1. In yet another embodiment, the gene expression panel or array comprises nucleic acid probes each of which is capable of hybridizing to an expression product of from 2-12 genes selected from the group consisting of ALDH6A1, PTDSS1, SCD, MYO5C, TFPI, PNMA2, TMEM45A, PLAT, HSD17B10, PLAT, EVI2B, and JAK1. Accordingly, cells of interest can be interrogated using an expression panel or array containing any combination of nucleic acid probes that are selective for genes KIAA1199 and NDRG1. Optionally, in addition to KIAA1199 and NDRG1, one or more genes from the group consisting of ALDH6A1, PTDSS1, SCD, MYO5C, TFPI, PNMA2, TMEM45A, PLAT, HSD17B10, PLAT, EVI2B, and JAK1 may be included in the panel or array. In one embodiment, the gene array or panel includes nucleic acids capable of hybridizing with a housekeeping gene, such as GAPDH.

The nucleic acids of the gene expression panel or array are about 10 to about 80 nucleotides in length; in some embodiments, about 20 to about 70 nucleotides in length.

In yet another aspect, the invention provides a plurality of polynucleotide probes, each complementary and hybridizable to a gene product of KIAA1199 and NDRG1 and one or more additional genes selected from ALDH6A1, SCD, MYO5C, TFPI, PNMA2, TMEM45A, PLAT, HSD17B10, PLAT, EVI2B, and JAK1. In one embodiment, the polynucleotide probes are immobilized on a solid support, such as, for example, a microarray.

In a related aspect, therefore, the invention relates to a gene expression panel or array indicative of the likelihood of relapse of breast cancer, said panel or array comprising or consisting essentially of primers and/or probes capable of evaluating the expression of genes, that is, capable of hybridizing under stringent conditions to expression products selected from the group consisting of ALDH6A1, NDRG1, PTDSSI, SCD, MYO5C, KIAA1199, TFPI, PNMA2, TMEM45A, PLAT, HSD17B10, PLAT, EVI2B, and JAK1.

In yet another aspect, the invention relates to a gene expression panel or array indicative of the likelihood of relapse/metastasis of breast cancer, said panel or array comprising or consisting essentially of primers and/or probes complementary to and capable of hybridizing under stringent conditions to an expression product of genes KIAA1199 and NDRG, thereby enabling the evaluation of the expression of genes KIAA1199 and NDRG1.

In one embodiment, the present invention is directed to a method of suppressing the metastatic ability of breast cancer cells, said method comprising: contacting said cells with a therapeutic agent, optionally in combination with other agents, targeted or not, that reduces the expression and/or activity of Coco in the cells.

In one embodiment, the invention is directed to a method for reducing the risk of relapse in a breast cancer patient comprising: identifying a patient at risk for relapse using the method(s) of the disclosure; and administering a therapeutic amount of an inhibitor of Coco or an agonist of bone morphogenic protein (BMP).

In yet another aspect, the invention relates to a method of identifying genes involved in metastasis and outgrowth, the method comprising: (a) transducing a poorly metastatic cell with a retroviral cDNA library from highly metastatic cells; (b) introducing transduced cells into a first site of a syngeneic non-human mammal; (c) recovering cells that have colonized a second site remote from the first site; (d) rescuing and sequencing integrated provirus recovered from a cell from said second site; and (e) identifying a cDNA from said provirus that confers a highly metastatic phenotype on poorly metastatic cells when said cDNA is introduced into untransduced cells.

### BRIEF DESCRIPTION OF THE DRAWINGS

FIGS. 1A-H show that the secreted BMP inhibitor, Coco, mediates mammary carcinoma metastasis to the lung. (A) is a schematic showing the gain-of-function retroviral cDNA screen. (B) shows a table that describes the metastatic capabilities of the mouse mammary carcinoma progression series used here and the results of the first series of screens. Note that the 4T1 cells express a limited number of cDNAs that can confer metastatic ability upon the 4T07 cells but not the 168FARN or 67NR cells. (C) The indicated cells were grown in serum-free medium. Proteins precipitated from supernatants (4 ml) or extracted from cell layers (30 μg) were subjected to immunoblotting with anti-Coco or anti-β-actin (top). Cells layers were treated with or without trypsin for 30 minutes, washed, lysed and then subjected to immunoblotting (bottom). (D) 4T07-TGL cells transduced with Coco-myc or empty vector (top) and 4T1 cells infected with lentiviral vectors encoding two shRNAs targeting Coco (sh #1 and #3) or a control shRNA (sh-Control) were subjected to immunoblotting as indicated. (E) 4T07-TGL cells transduced with Coco or not were inoculated into the fourth mammary fat pads of syngeneic mice. Tumor volumes were calculated at the indicated time points by caliper measurement. Each data point represents the mean±SD. (F) 4T1 cells infected with lentiviral vectors encoding two shRNAs targeting Coco (sh #1 and #3) or a control shRNA (sh-Control) were inoculated into the fourth mammary fat pads of syngeneic mice. Tumor volumes were calculated at the indicated time points by caliper measurement. Each data point represents the mean±SEM. (G) 4T07-TGL cells stably expressing Coco or not were inoculated intravenously into syngeneic mice. Lung metastasis was measured by bioluminescent imaging. The panels show representative images (left) and the graph shows the normalized photon flux at the indicated times (right). Note that the scale for normalized photon flux is logarithmic. (H) 4T1 cells infected with lentiviral vectors encoding a control shRNA (sh-Control) or two shRNAs targeting Coco (sh #1 and #3) were inoculated intravenously into syngeneic mice. Lung metastasis was measured by bioluminescent imaging. The panels show representative images (left) and the graph shows the normalized photon flux at the indicated times (right).

FIGS. 2A-J show that the secreted BMP inhibitor Coco induces solitary tumor cells to exit from dormancy in the lung. (A) 4T07-TGL cells stably expressing Coco or not were inoculated intravenously into syngeneic mice. Mice were sacrificed at the indicated times and 50 μm lung sections were processed for immunofluorescent detection of GFP (tumor cells) and PECAM-1 (endothelial cells). (B) The graph shows the number of GFP-positive solitary tumor cells per microscopic field (left vertical axis) and of metastatic lesions per lung section (right vertical axis) at the indicated times in mice injected with 4T07-TGL cells transduced with empty vector (top) or stably expressing Coco (bottom). Metastatic outgrowths could only be detected in the lungs of mice injected with 4T07 cells expressing Coco (red bars; P<5.6E-4 at 21 days and 3.1E-05 at 35 days). (C) Outline of the EdU incorporation assay. 4T07-TGL cells stably expressing Coco or not were inoculated intravenously into syngeneic mice at day 0. Mice were injected for 3 consecutive days with EdU and sacrificed at the indicated times. (D) Lung sections were processed for simultaneous detection of GFP (tumor cells) and EdU. The panels show representative images. (E) The graph shows the percentage of control 4T07 cells or 4T07 cells expressing Coco exhibiting nuclear EdU staining at the indicated times. Cells expressing Coco were categorized as solitary (S) or outgrowing (O). Total values (T) were calculated by combining the values for solitary and outgrowing cells. **p<0.01. (F) 4T1 cells infected with lentiviral vectors encoding a control shRNA (sh-Control) or two shRNAs targeting Coco (sh #1 and #3) were inoculated intravenously into syngeneic mice. Lung sections were subjected to double immunostaining with antibodies to Ki-67 and GFP. Control cells were categorized as solitary (S) or outgrowing (O). The graph shows the percentage of cells exhibiting nuclear anti-Ki-67 staining at the indicated times. **p<0.01. (G) Semiquantitative RT-PCR analysis of BMP receptors in 4T07 and 4T1 cells (top). 4T07 cells stably expressing Coco or not were treated with or without BMP4 (1 ng/ml) for 30 minutes and subjected to immunoblotting with anti-P-Smad (1,5,8) antibody (bottom). (H) 4T07-TGL cells stably expressing Coco or not were inoculated intravenously into syngeneic mice. Mice were sacrificed at the indicated times and lung sections were subjected to double immunostaining with anti-P-Smad (1,5,8) and anti-GFP. The graph shows the percentage of P-Smad (1,5,8) negative 4T07 tumor cells at the indicated times. Cells expressing Coco were categorized as solitary or outgrowing. Metastatic outgrowths could only be detected in the lungs of mice injected with 4T07 cells expressing Coco (red bars). (I) The panels show the positive staining pattern generated by the anti-P-Smad (1,5,8) antibody on dormant tumor cells present in the lungs of mice injected with control 4T07-TGL cells and the lack of reactivity of the same antibody with a subpopulation of dormant tumor cells present in the lungs of mice injected with 4T07-TGL cells expressing Coco. Metastatic lesions arising in mice injected with 4T07-TGL cells stably expressing Coco were counterstained with hematoxylin (bottom right). (J) The indicated cells were inoculated intravenously into syngeneic mice. Lung sections were subjected to double immunostaining with antibodies to P-Smad (1,5,8) and GFP. Control cells were categorized as solitary (S) or outgrowing (O). The graph shows the percentage of cells negative for P-Smad staining at the indicated times.

FIGS. 3A-J show that Coco promotes formation of tumor spheres in vitro and tumor initiation in vivo. (A) 4T07-TGL cells stably expressing Coco or not were subjected to tumor sphere assay. At weekly intervals, the cells were dissociated, counted, and replated. The graph shows the number of tumorspheres formed at each of three subsequent passages per 10³ cells seeded. (B) 4T07 cells were plated in the presence of the indicated concentrations of recombinant Coco and subjected to tumor sphere assay. SDS-PAGE and Coomassie Blue staining were used to assess the purity of recombinant Coco (left). The graph shows the number of tumor spheres formed per 10³ cells seeded (right). (C) 4T07 cells were subjected to tumor sphere assay in medium supplemented with 10 ng/ml BMP4 combined with the indicated concentrations of recombinant Coco. The graph shows the efficiency of tumor sphere formation as compared to 4T07 control cells. ***p<0.001. (D) The pictures show images of tumor spheres generated by 4T07 cells left untreated or treated with 0.9 μg/ml Coco, 10 ng/ml BMP4, or the combination of the two. (E) Outline of PKH-26 staining and serial tumor sphere assay. ErbB2-transformed cells were stained with PKH-26, examined by FACS (left), and subjected to primary tumor sphere assay. The primary spheres were dissociated and their constituent cells were sorted according to staining intensity (PKH^{HIGH}, PKH^{LOW}, and PKH^{NEG}) (center) and subjected to secondary tumor sphere assays. Finally, the PKH^{HIGH} cells from secondary tumor spheres were subjected to the same protocol to derive tertiary tumor spheres. (F) Control and Coco-silenced ErbB2-transformed cells were subjected to staining with PKH-26 and serial tumor sphere assay as outlined above. The graph shows the number of tumor spheres formed at each passage by the indicated subpopulations of cells. *p<0.05, **p<0.01, ***p<0.001, NS—not significant. (G) 4T07 cells stably expressing Coco and relative controls were inoculated into the fourth mammary fat pad of syngeneic mice. Numbers of cells injected were as follows: 1×10⁴ (left), 5×10⁴ (middle), and 2×10⁵ (right). Tumor volumes were calculated at the indicated time points by calliper measurement. Each data point represents the mean±SEM. The right graph was designed by replotting the results of FIG. 1E. (H) 4T1 cells infected with lentiviral vectors encoding two shRNAs targeting Coco (sh #1 and #3) or a control shRNA (sh-Control) were inoculated into the fourth mammary fat pad of syngeneic mice. Numbers of cells injected were as follows: 1×10³ (left), 1×10⁴ (middle), and 1×10⁵ (right). Tumor volumes were calculated at the indicated time points by calliper. Each data point represents the mean±SEM. The right graph was designed by replotting the results of FIG. 1F. (I and J) The indicated cell lines were subjected to Q-PCR analysis of Nanog, Sox2, Oct4 and Taz expression. BMP4 was used at 10 ng/ml for 2 days.

FIGS. 4A-E show the inhibition of BMP signaling formation of tumor spheres and lung colonization. (A) 4T07 cells were transduced with retroviral vectors encoding inhibitory Smad6, stabilized β-catenin (β-cat-S4A), or both signaling proteins (Sm6/βcat) and subjected to immunoblotting with the indicated antibodies (left) and to a tumor sphere-forming assay. The graph shows the number of tumor spheres formed per 103 cells seeded (right). (B) The panels show representative pictures for (A). (C) 4T07 cells stably transduced with retroviral vectors encoding Smad6 or β-catenin-S4A or with a control vector were inoculated in the tail vein of syngeneic mice. Lung metastasis was measured by bioluminescent imaging. The panels show representative images (top) and the graph shows the normalized photon flux at the indicated times (bottom). (D) 4T1 cells stably transduced with a retroviral vector encoding a constitutively activated BMPR (CA-BMPR) or with a control vector were inoculated into the fourth mammary fat pad of mice. Numbers of cells injected were as follows: 1×10³ (top), 1×10⁴ (middle), 1×10⁵ (bottom). Tumor volumes were measured at the indicated times by caliper. The graph shows the mean±SD. (E) 4T1 cells stably transduced with a retroviral vector encoding a constitutively activated BMPR (CA-BMPR) or with a control vector were inoculated into the tail vein of syngeneic mice. Lung metastasis was measured by bioluminescent imaging. The panels show representative images (top) and the graph shows the normalized photon flux at the indicated times (bottom).

FIGS. 5A-H show that expression of Coco promotes human breast cancer metastasis. (A) 12 human breast cancer cell lines classified according to tumorigenic potential, ability to invade through Matrigel in vitro, expression of a stem cell and mesenchymal phenotype, metastatic ability (Bo: bone only; L*: predominantly lung), transcriptomic subtype (BaA and BaB: basal A and B; Lu: luminal), ER and HER2 status were subjected to immunoblotting with anti-Coco and anti-β-actin. (B) MDA-MB-231 cells stably transduced with lentiviral vectors encoding a control sh-RNA (sh-Control) or two sh-RNAs targeting Coco (sh-Coco #2 and #4) were subjected to immunoblotting with the indicated antibodies (top left) and inoculated into the tail vein of nude mice. Lung metastasis was measured by bioluminescent imaging. The graph shows the normalized photon flux at the indicated times (bottom left) and the panels show representative images (right). (C) MDA-MB-231 cells stably transduced with lentiviral vectors encoding a control sh-RNA (sh-Control) or two sh-RNAs targeting Coco (sh-Coco #2 and #4) were subjected to FACS analysis with antibodies to CD44 and CD24. (D) MDA-MB-231 cells stably transduced with lentiviral vectors encoding a control sh-RNA (sh-Control) or two sh-RNAs targeting Coco (sh-Coco #2 and #4) were subjected to tumor sphere assay. The graph at the left shows the number of tumor spheres formed per 10³ cells seeded and the graph at the right the number of viable cells at the end of the assay as measured by MTT staining. (E) MDA-MB231 cells infected with lentiviral vectors encoding two shRNAs targeting Coco (sh #2 and #4) or a control shRNA (sh-Control) were inoculated into the fourth mammary fat pad of NOD/SCID/IL2Rγ−/− mice. Numbers of cells injected were as follows: 1×10³ (left), 1×10⁴ (middle), and 1×10⁶ (right). Tumor volumes were calculated at the indicated time points by calliper. Each data point represents the mean±SEM. *p<0.05, **p<0.01, n.s.—not significant. (F) Normal breast tissue and a case of invasive ductal carcinoma were subjected to staining with monospecific antibodies to Coco followed by counterstaining with Hematoxylin. Note the positivity of nests of tumor cells and scattered stromal cells (high magnification inset). (G) Tissue microarrays comprising 126 primary breast tumors were subjected to staining with monospecific antibodies to Coco. The pictures show representative images of cases exhibiting varying levels of positivity. The graph shows the frequency distribution of staining intensity across all samples. (H) Kaplan-Meier analysis of overall survival based on Coco expression in primary tumors. Patients were divided according to the intensity of Coco staining.

FIGS. 6A-H show a Coco gene expression signature predicts metastatic relapse. (A) Triplicate samples of MDA-MB-231 cells stably transduced with lentiviral vectors encoding a control sh-RNA (sh-Control) or two sh-RNAs targeting Coco (sh-Coco #2 and #4) were subjected to DNA microarray analysis. The genes that were concordantly upregulated or downregulated (>2 fold) in Coco-silenced cells as compared to controls were subjected to hierarchical clustering. Components of the 2-gene signature are underlined. (B) Kaplan-Meier analysis of metastasis-free survival in the NKI295 dataset (left) or MSK82, EMC192 and EMC286 combined dataset (right). Patients were divided according to the expression of a 14-gene Coco signature (red line, positive; blue line, negative). (C) MDA-MB231 cells and their lung and bone metastatic derivatives were subjected to immunoblotting with anti-Coco and anti-β-actin or anti-α-tubulin. (D) Kaplan-Meier analysis of lung only metastasis-free survival (left), bone only metastasis-free survival (middle), and brain only metastasis-free survival (right) in the MSK82, EMC192 and EMC286 combined dataset. Patients were divided according to the expression of a 14-gene Coco signature (red line, positive; blue line, negative). (E) Kaplan-Meier analysis of lung only metastasis-free survival (left), bone only metastasis-free survival (middle), and brain only metastasis-free survival (right) in the MSK82, EMC192 and EMC286 combined dataset. Patients were divided according to the expression of a 2-gene Coco signature (red line, positive; blue line, negative). (F) MDA-MB-231 cells stably transduced with lentiviral vectors encoding a control sh-RNA (sh-Control) or two sh-RNAs targeting NDRG1 (sh-NDRG #3 and #5) were inoculated into the tail veins of nude mice. Lung metastasis was measured by bioluminescent imaging. The graph shows the normalized photon flux at the indicated times. (G) MDA-MB-231 cells stably transduced with lentiviral vectors encoding a control sh-RNA (sh-Control) or two sh-RNAs targeting KIAA1199 (sh-KIAA #1 and #5) were inoculated into the tail veins of nude mice. Lung metastasis was measured by bioluminescent imaging. The graph shows the normalized photon flux at the indicated times. (H) MDA-MB-231 cells stably transduced with lentiviral vectors encoding a control sh-RNA (sh-Control) or sh-RNAs targeting NDRG1 and KIAA1199 (sh-KIAA #1/NDRG #3 and KIAA #5/NDRG #5) were inoculated into the tail veins of nude mice. Lung metastasis was measured by bioluminescent imaging. The graph shows the normalized photon flux at the indicated times (left) and the panels show representative images (right).

FIG. 7A-E show that Coco-mediated blockage of BMP is not required for metastasis to the bone or brain. (A) 4T1 cells infected with lentiviral vectors encoding a control shRNA (sh-Control) or two shRNAs targeting Coco (sh #1 and #3) were inoculated intracardiacally in syngeneic mice. Bone, brain and adrenal gland metastases were measured by bioluminescent imaging. Bone metastases were confirmed by X-ray radiography. The panels show representative images. Yellow dotted lines delineate the boundaries of osteolytic lesions in the radiograms. (B) The graph shows the photon flux signal of metastases in the hind limbs, brains and adrenal glands of mice inoculated with indicated cell lines. (C) 4T1-TGL cells were inoculated intracardiacally into syngeneic mice and sacrificed 7 days later. Bone and brain sections were subjected to double immunostaining with anti-P-Smad 1,5,8 and anti-GFP. The graph shows the percentage of P-Smad+ and P-Smad− tumor cells present in the bones (left column) and the brains (right column). The images show examples of P-Smad− and P-Smad+ solitary tumor cells present in the bone (top and middle right) and of P-Smad− solitary tumor cells present in the brain (bottom right). (D) 4T1 and 4T07 cells were inoculated intracardiacally into syngeneic mice. Mice were sacrificed at the indicated times. Parallel bone sections were subjected to H&E staining or immunostaining with anti-P-Smad 1,5,8. The graph shows the percentage of P-Smad+ and P-Smad− tumor cells in micrometastases and macroscopic metastatic lesions generated by 4T1 cells and in micrometastatic clusters generated by 4T07 cells (top left). The pictures show representative images. Insets show anti-P-Smad staining. (E) The model illustrates the mechanism underlying the capacity of Coco to promote exit from dormancy at lung metastatic sites. BMP proteins produced by lung-resident cells inhibit the ability of tumor progenitor cells that have extravasated in the lung stroma to undergo self-renewal and thereby induce them to enter into proliferative quiescence. Pericellular accumulation of Coco shields tumor progenitor cells from the inhibitor action of BMP proteins.

FIGS. 8A-N show that Coco promotes the homing and outgrowth step of metastasis in multiple mouse models. (A) 4T07 cells expressing the TGL vector encoding luciferase (4T07-TGL) were transduced with the retroviral pEYK vector encoding a FLAG-tagged form of cDNA1 or with empty vector, subjected to immunoblotting with anti-FLAG or anti-β-actin (left), and inoculated in the fourth mammary fat pads of syngeneic mice. Lung colonization was monitored by bioluminescent imaging over 7 weeks. At the end of the experiment, 2 out of 5 mice injected with 4T07-TGL cells expressing FLAG-cDNA1 had developed lung metastases (middle left). Primary tumor growth by weight (middle right) and lung colonization was quantified by bioluminescent imaging (left). Note that the scale for normalized photon flux is logarithmic. (B) Structure of the cDNA isolated from the in vivo metastasis screen. The cDNA corresponds to Dante, a fusion transcript first described by Rossant and colleagues during studies on early mouse development (Pearce et al., 1999). Dante does not contain sequences encoding an initiator Met residue and RACE experiments have failed to extend its sequence at the 5′ end (Pearce et al., 1999) (data not shown). Whereas the first 69 nucleotides of the transcript (light purple) are encoded by a sequence present on the non-coding strand of the GADD45gip1 gene located approximately 10 Kb upstream of the gene encoding Coco (DAND5), the remaining part (dark purple) is encoded by exon 2 of DAND5 and directs the synthesis of the cysteine knot domain of Coco, which is thought to mediate inhibition of BMP proteins. Vector sequences are in green. An antibody directed to a peptide comprising the amino acid sequence encoded by the 69 nucleotides that distinguish Dante from the mRNA encoding Coco reacts well in blotting with the recombinant protein encoded by the vector containing the fusion transcript but fails to detect an endogenously expressed protein in 4T1 cells, although these cells express the fusion transcript (data not shown). Furthermore, the human and the frog genomes do not contain a sequence homologous to the 69 nucleotides that encode the unique 5′ portion of Dante. (C) 4T07-TGL transduced with pEYK encoding FLAG-cDNA1 or empty vector were inoculated in the tail vein of syngeneic mice. Lung colonization was measured by bioluminescent imaging. The graph shows the normalized photon flux at the indicated times (left). The panels show representative images (right). (D) Transcription assays for BMP, Nodal and Wnt activity. 4-cell stage Xenopous embryos were injected with the indicated RNAs and luciferase activity was measured in extracts from stage 10 embryos. Left: activity of the BRE reporter gene induced by BMP4. Middle: activity of the A3Luc reporter gene induced by Nodal (Xnr1). Right: activity of the TOP-FLASH reporter gene induced by Wnt 8. (E) Ventral injection of 1 ng of the indicated constructs in 4-cell stage embryos. The pictures show tadpole stage embryos. The arrow indicates the partial secondary axis. (F) cDNA1 expressed as a secreted protein in 4T07 cells. Cell layers that were exposed to trypsin for the indicated times were subjected to immunoblotting with anti-FLAG and anti-β-actin. (G) 4T07 cells stably transduced with control vector or cDNA1 were treated with or without BMP4 (1 ng/ml) for 30 minutes and subjected to immunoblotting with anti-pSmad 1, 5, 8 and anti-Smad 1. (H) 4T07-TGL cells stably expressing Coco or empty vector were inoculated in the fourth mammary fat pads of syngeneic mice. Lung colonization was monitored by bioluminescent imaging over 7 weeks. At the end of the experiment, 2 out of 5 mice injected with 4T07-TGL cells expressing Coco had developed lung metastases. Lung colonization was quantified by bioluminescent imaging. Note that the scale for normalized photon flux is logarithmic. (I) 4T07 cells stably transduced with a control vector or Coco were cultured in DMEM with 10% FBS. Triplicate samples were counted at the indicated times. (J) NMuMG cells stably transduced with a control vector or Coco were cultured in DMEM with 10% FBS for 2 days, and then subjected to double staining with anti-β-cat and anti-ZO-1 followed by counterstaining with DAPI (left) or to immunoblotting with antibodies to epithelial proteins, including E-cadherin, α-catenin, β-catenin, and γ-catenin, and mesenchymal proteins including fibronectin, vimentin, and N [symbol] cadherin. β-actin was used as a loading control (right). (K) 4T07 cells stably transduced with a control vector or Coco were cultured in DMEM with 10% FBS for 2 days, and then subjected to staining with anti-E-cadherin (E-cad), anti-β-catenin (13-cat), and anti-ZO-1 followed by counterstaining with DAPI or to immunoblotting as in (J). (L) 4T07 cells stably transduced with a control vector or expressing Coco were subjected to Matrigel invasion assay in response to FBS. The graph shows the mean number of invaded cells (±SD) per transwell insert from triplicate samples. (M) 66cl4 cells infected with lentiviral vectors encoding two shRNAs targeting Coco (sh #1 and #3) or a control shRNA (sh-Control) were subjected to immunoblotting as indicated (left top) and were inoculated intravenously in syngeneic mice. Lung metastasis was measured by bioluminescent imaging. The graph shows the normalized photon flux at the indicated times (left bottom) and the pictures show representative images (right). (N) ErbB2-transformed mammary tumor cells from MMTV-Neu(YD) mice were infected with lentiviral vectors encoding two shRNAs targeting Coco (sh #1 and #3) or a control shRNA (sh-Control) and subjected to immunoblotting as indicated (left top) or were inoculated intravenously into nude mice. Lung metastasis was measured by bioluminescent imaging. The graph shows the normalized photon flux at the indicated times (left bottom) and the pictures show representative images (right).

FIGS. 9A-N show phenotypic and functional characterization of extravasated tumor cell. (A) Mice were inoculated intravenously with 4T07 cells expressing Coco or not. Lung sections obtained at the indicated times were subjected to double immunostaining with antibodies to Ki-67 and GFP or to single immunostaining with anti-Ki67 followed by counterstaining with Hematoxylin. The pictures show representative images of control and Coco-expressing solitary tumor cells as well as metastatic lesions arising from Coco-expressing cells. (B) The graph shows the percentage of control 4T07 cells or 4T07 cells expressing Coco exhibiting positivity for anti-Ki67 staining at the indicated times. Cells expressing Coco were categorized as solitary (S) or outgrowing (O). Total values (T) were calculated by combining the values for solitary and outgrowing cells. *p<0.05, **p<0.01. (C) Lung sections from mice that had been injected intravenously with 4T07-TGL cells stably expressing Coco or not as described in FIG. 2G were subjected to double immunostaining with antibodies to cleaved caspase 3 and GFP. Metastatic lesions arising in mice injected with 4T07-TGL cells stably expressing Coco were stained with an antibody to cleaved caspase 3 followed by counterstaining with hematoxylin. Isolated apoptotic cells detected within primary tumors generated by 4T07 cells served as a positive control. (D) 4T1 cells infected with lentiviral vectors encoding a control shRNA (sh-Control) or two shRNAs targeting Coco (sh #1 and #3) were inoculated intravenously into syngeneic mice. Lung sections were subjected to immunostaining with antibody to GFP. The graphs show the number of GFP-positive solitary tumor cells per microscopic field (left) and of metastatic lesions per lung section (right) at the indicated times. (E) Semiquantitative RT-PCR was used to examine expression of Nodal receptors and co-receptor Cripto in 4T07 and 4T1 cells. (F) 4T07 cells were transfected with the Nodal-responsive luciferase reporter gene A3Luc, treated with the indicated amounts of Nodal, and subjected to luciferase assay. (G) Semiquantitative RT-PCR was used to examine expression of Wnt receptors and co-receptors in 4T07 and 4T1 cells. (H) 4T07 cells stably expressing a control vector or Coco were treated with Wnt-3A for 3 hrs at the indicated concentrations. Expression of β-catenin or Coco was examined by immunoblotting with anti-β-cat or anti-myc antibody. β-actin was used as a loading control. (I) 4T07 cells stably expressing a control vector or Coco were treated with Wnt3A for 3 hrs at the indicated concentrations, and then subjected to staining with anti-β-catenin followed by counterstaining with DAPI. (J) The graph shows the percentage of nuclear β-catenin-positive cells. (K) 4T07 cells expressing a control vector or Coco were transfected with the Wnt-responsive luciferase reporter gene TOP-FLASH, treated with the indicated amounts of Wnt1, and subjected to luciferase assay. (L) Identical amounts of total RNA extracted from mouse lung, 4T07 and 4T1 cells, mammary fat pad, and the indicated primary tumors were subjected to semiquantitative RT-PCR to examine the level of expression of the indicated BMP proteins, BMP15 (which acts as an inhibitor of BMP signalling (Di Pasquale and Brivanlou, 2009) and GDF5. GAPDH was used as a control. (M) Lung sections were subjected to immunohistochemical staining for β-catenin and GFP. Isolated and outgrowing tumor cells lacking nuclear β-catenin were quantified at the indicated times. (N) Representative picture of metastatic lesion generated by 4T07 cells expressing Coco stained with anti-β-catenin and counterstained with Hematoxylin.

FIGS. 10A-M show that Coco promotes the manifestation of stem cell traits. (A) The expression of stem cell markers CD24, CD29, and CD49f was examined by FACS analysis of the indicated cells. (B) Representative images of tumor spheres formed by 4T07 cells expressing Coco or not. (C) 4T07 cells were treated with Coco, BMP4, or Coco and BMP4, stained with PI and subjected to FACS analysis. (D) ErbB2-transformed cells were subjected to tumor sphere assay in medium supplemented with 50 ng/ml BMP4 or the combination of 50 ng/ml BMP4 and 0.9 μg/ml Coco. The graph shows the number of tumor spheres formed per 10³ cells seeded. (E) Control, Coco-silenced, and BMP4-treated ErbB2-transformed cells were stained with PI and subjected to FACS analysis. (F) 4T07 cells were treated with the indicated concentrations of BMP4, alone or in combination with 300 ng/ml Coco, and subjected to immunoblotting with the indicated antibodies. (G) Lung sections from mice injected intravenously with 4T07 cells expressing Coco or not were subjected to immunohistochemical staining for GATA3 followed by counterstaining with Hematoxylin. Solitary dormant and outgrowing tumor cells exhibiting nuclear staining for GATA3 were quantified at the indicated times (left). The pictures show the positive staining pattern generated by anti-GATA3 antibodies on dormant tumor cells at the indicated times and the negative staining of metastatic lesions arising in mice injected with 4T07 cells expressing Coco (right). ***p<0.001. (H) 4T07 cells stably expressing a control vector or Coco were subjected to clonogenic assay. The graph shows the mean number of colonies (±SD) per 1000 cells from triplicate samples (left). The pictures show representative plates (middle) and individual colonies (right). (I) 4T07 cells stably expressing a control vector or Coco were subjected to soft agar assay. The graph shows the mean number of colonies (±SD) per field from triplicate samples. (J and K) The relative expression levels of Nanog, Sox2, Oct4 and Taz were examined by Q-PCR in the indicated cell. (L) Control and Coco-silenced ErbB2-transformed cells were subjected to Q-PCR analysis of Nanog, Sox2, Oct4 and Taz expression. (M) ErbB2-transformed cells were cultured in 3D Matrigel in the presence or the absence of 100 ng/ml BMP4 for 7 days and were subjected to Q-PCR analysis.

FIGS. 11A-I shows that inhibition of BMP signaling inhibits commitment to differentiation and promotes lung colonization. (A) 4T07 cells stably expressing Smad6 or DN-BMPR-IB (K231R) were subjected to immunoblotting as indicated. (B) 4T07 cells stably expressing Smad6, DN-BMPR-IB (K231R) or not were treated with BMP4 at the indicated concentrations and subjected to immunoblotting as indicated. (C) 4T07 cells stably transduced with a retrovirus encoding β-cat-S4A or a control vector were subjected to immunoblotting as indicated. (D) 4T07 cells transduced with a control vector or activated β-catenin were transfected with TOP-FLASH, treated with Wnt1 at the indicated concentrations, and subjected to TOP-FLASH luciferase assay. (E) 4T07 cells stably transduced with a retrovirus encoding Smad6 or control vector were cultured for 48 hours in serum-free medium supplemented with the indicated concentrations of BMP4 and subjected to immunoblotting with the indicated antibodies. (F) 4T07-TGL cells stably expressing DN-BMPR-IB (K231R) and relative controls were inoculated intravenously into syngeneic mice. Lung metastasis was measured by bioluminescent imaging. The graph shows the normalized photon flux at the indicated times (left) and the panels representative images (right). (G) Coco-silenced 4T1 cells (4T1-shCoco #1 and 4T1-shCoco #3) as well as their derivatives co-expressing DN-BMPR-IB (K231R) were inoculated intravenously into syngeneic mice. Lung metastasis was measured by bioluminescent imaging. The graph shows the normalized photon flux at the indicated times. (H) 4T1 cells stably co-transduced with retroviruses encoding Myc-BMPR-IB(Q203D) and Flag-BMPR-II (collectively indicated as CA-BMPR) or a control vector were subjected to immunoblotting as indicated. (I) 4T07-Coco cells stably expressing CA-BMPR and relative controls were inoculated intravenously into syngeneic mice. Lung metastasis was measured by bioluminescent imaging. The graph shows the normalized photon flux at the indicated times (left) and the panels representative images (right).

FIGS. 12A-I show that Coco promotes human breast cancer metastasis to lung. (A) Q-PCR analysis of Cerberus1, Chordin, Chordin-like1, Chordin-like2, DAN, Gremlin, Gremlin2, Noggin, and SOSTDC1 in 12 human breast cancer cell lines. Note that the scale of the y-axis is different for DAN and SOSTDC1. (B) Q-PCR analysis of Cerberus1, Chordin, Chordin-like2, Gremlin, Gremlin2, and SOSTDC1 in MDA-MB231 cells and Coco, Chordin-like1, DAN and Noggin in MDA-MB-231 cells stably transduced with lentiviral vectors encoding a control sh-RNA (sh-Control) or two sh-RNAs targeting Coco (sh-Coco #2 and #4). (C) Immunoblotting was used to estimate the relative levels of expression of Coco, Noggin, and DAN in 30 μg of total proteins from MDA-MB231 cells. The indicated amounts of purified recombinant human Coco, Noggin, and a DAN-IgG1 fusion protein were used as reference. DAN-IgG1 has a larger molecular weight as compared to wild type DAN in MDA-MB231 cells. The two samples were run alongside each other, but the lanes are shown separated by a dotted line because they were realigned to allow direct comparison of the intensity of the two bands. The graph shows the concentration of each inhibitor in MDA-MB231 lysates as estimated by densitometry (bottom right). (D) MDA-MB-231 cells stably transduced with lentiviral vectors encoding a control sh-RNA (sh-Control) or two sh-RNAs targeting Coco (sh-Coco #2 and #4) were inoculated into the tail veins of nude mice. Lung metastasis was measured by bioluminescent imaging. The graph shows the normalized photon flux at the indicated times (left). The 0 to 10 weeks datapoints were derived from the results of FIG. 5B. Control mice had to be sacrificed at 10 weeks because of metastatic burden. Note that the vertical axis is linear. Sections of lung metastatic lesions were subjected to immunohistochemical staining with affinity-purified antibodies to Coco. The panels show representative pictures (right). (E) MCF7 cells stably expressing Myc-tagged Coco or control vector were subjected to immunoblotting with anti-Myc or anti-β-actin (left). The indicated cells were inoculated in the fourth mammary fat pad of nude mice. Sections of primary tumors were subjected to immunohistochemical staining with affinity-purified antibodies to Coco. The panels show representative pictures (middle and right). (F) CN34.2a cells infected with lentiviral vectors encoding two shRNAs targeting Coco (sh #2 and #4) or a control shRNA (sh-Control) were subjected to immunoblotting as indicated (top left) and were inoculated intravenously into NOD/SCID/IL2Rγ−/− mice. Lung metastasis was measured by bioluminescent imaging. The graph shows the normalized photon flux at the indicated times (left bottom) and the pictures show representative images (right). (G) MDA-MB231 cells infected with lentiviral vectors encoding two shRNAs targeting Coco (#2 and #4) or a control shRNA (sh-Co.) were subjected to BrdU incorporation assay to estimate their rate of proliferation in vitro. (H) The panels show representative images of control and Coco-silenced MDA-MB231 cells subjected to tumor sphere assay. (I) Low magnification images of tissue microarray cores comprising cases characterized by varying degrees of Coco positivity.

FIGS. 13A-G show derivation and validation of the 14-gene and 2-gene Coco signatures. (A) Kaplan-Meier analysis of metastasis-free survival in the EMC286 datasets. Patients were divided according to the expression of a 14-gene signature (red line, positive; blue line, negative). (B) Kaplan-Meier analysis of metastasis-free survival in the NKI295, EMC286 and MSK82+EMC192+EMC286 combined datasets. Patients were divided according to the expression of a 2-gene signature (red line, positive; blue line, negative). (C) Kaplan-Meier analysis of lung metastasis-free survival, bone metastasis-free survival, and brain metastasis-free survival in the MSK82, EMC192 and EMC286 datasets. Patients were divided according to the expression of a 14-gene or a 2-gene Coco signature (red line, positive; blue line, negative). (D) Kaplan-Meier analysis of metastasis-free survival, lung metastasis-free survival, bone metastasis-free survival, and brain metastasis-free survival in the combined MSK82+EMC192+EMC286+NKI295 dataset. Patients were divided according to the expression of a 56-gene signature (red line, positive; blue line, negative). The association with brain relapse may be due to the frequent co-occurrence of lung and brain metastasis in the unselected cohort. (E) MDA-MB-231 cells stably transduced with lentiviral vectors encoding a control sh-RNA (sh-Control) or two sh-RNAs targeting NDRG1 (sh-NDRG #3 and #5) were subjected to immunoblotting as indicated (left) and inoculated into the tail veins of 5 nude mice. Lung metastasis was measured by bioluminescent imaging. The pictures show representative images (right). (F) MDA-MB-231 cells stably transduced with lentiviral vectors encoding a control sh-RNA (sh-Control) or two sh-RNAs targeting KIAA1199 (sh-KIAA #1 and #5) were subjected to immunoblotting as indicated (left) and inoculated into the tail veins of nude mice. Lung metastasis was measured by bioluminescent imaging. The pictures show representative images (right). (G) MDA-MB-231 cells stably transduced with lentiviral vectors encoding a control sh-RNA (sh-Control) or sh-RNAs targeting NDRG1 and KIAA1199 (sh-KIAA #1/NDRG #3 and KIAA #5/NDRG #5) were subjected to immunoblotting.

FIGS. 14A-B show histological confirmation of bone metastasis and validation of P-Smad staining of bone sections. (A) Sections of the rear limb from a control mouse (left) or one inoculated 5 weeks earlier intracardiacally with 4T1 cells (right) were subjected to H&E staining. The yellow dotted line demarcates the boundary of an osteolytic lesion. (B) Parallel sections of the rear limb of a control mouse were subjected to staining with H&E (left) or anti-P-Smad 1,5,8 antibodies (right). The arrows point to chondrocytes exhibiting accumulation of P-Smad 1,5,8 in their nuclei.

### DETAILED DESCRIPTION OF THE INVENTION

All patents, published applications and non-patent references cited in this disclosure are hereby expressly incorporated herein by reference in their entirety.

In practicing the present invention, many conventional techniques in molecular biology are used, which are within the skill of the ordinary artisan. These techniques are described in greater detail in, for example, Molecular Cloning: a Laboratory Manual 3rd edition, J. F. Sambrook and D. W. Russell, ed. Cold Spring Harbor Laboratory Press 2001; “Oligonucleotide Synthesis” (M. J. Gait, ed., 1984); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988) and DNA Microarrays: A Molecular Cloning Manual. D. Bowtell and J. Sambrook, eds. Cold Spring Harbor Laboratory Press 2002. The contents of these references and other references containing standard protocols, widely known to and relied upon by those of skill in the art, including manufacturers' instructions are hereby incorporated by reference as part of the present disclosure.

Technical and scientific terms used herein are intended to have their normal meaning as commonly understood by one of ordinary skill in the art to which this invention pertains.

Therapeutic agents for practicing a method of the present invention may include inhibitors of Coco expression or Coco activity. An “inhibitor,” as that term is known in the art, is any substance which retards, prevents or otherwise interferes with a chemical, physiological or biological reaction or activity. Common inhibitors include but are not limited to antisense molecules, siRNAs, miRNAs, shRNAs, antibodies, and other antagonists.

Oligonucleotide “probes” have long been used to detect complementary nucleic acid sequences in a nucleic acid of interest (the “target” nucleic acid). In some assay formats, the oligonucleotide probe is tethered, i.e., by covalent attachment, to a solid support, and arrays of oligonucleotide probes immobilized on solid supports have been used to detect specific nucleic acid sequences in a target nucleic acid.

“Probes” may be derived from naturally occurring or recombinant single- or double-stranded nucleic acids or may be chemically synthesized. They are useful in detecting the presence of identical or similar sequences. Nucleic acid probes used in practicing the disclosed methods can be designed based on the sequence of a gene encoding a known protein. One skilled in the art can readily design such probes based on the known sequence using methods of computer alignment and sequence analysis known in the art (e.g. “Molecular Cloning: A Laboratory Manual”, second edition, edited by Sambrook, Fritsch, & Maniatis, Cold Spring Harbor Laboratory, 1989; Ausubel, et al, Short Protocols in Molecular Biology, 3rd ed. 1995). The probe can comprise any number of nucleotides but will preferably be not fewer than 10 nucleotides and preferably not more than about 80 nucleotides in length; in some embodiments, probe length will be about 20 to 60 nucleotides; in still other embodiments, probe length will be about 20 to 40 nucleotides.

The probes of the invention can be labeled by standard labeling techniques such as with a radiolabel, enzyme label, fluorescent label, biotin-avidin label, chemiluminescent label, and the like in accordance with methods known to those of skill in the art. After hybridization, the probes may be detected using known methods.

Nucleic acid probes of the present invention include RNA and DNA probes as well as nucleic acids modified in the sugar, phosphate or even the base portion as long as the probe retains the ability to specifically hybridize under conditions as disclosed herein. Such probes are generated using techniques known in the art.

“Hybridization” refers to a reaction in which one or more polynucleotides or nucleic acids react to form a complex that is stabilized via hydrogen bonding between the bases of the nucleotide residues. The hydrogen bonding may occur by Watson-Crick base pairing, or in any other sequence-specific manner. If a nucleic acid binds to the DNA or RNA molecule with sufficiently high affinity, it is said to “hybridize” to the DNA or RNA molecule. The strength of the interaction between the probing sequence and its target can be manipulated by varying the stringency of the hybridization conditions. Various low to high stringency hybridization conditions may be used depending upon the specificity and selectivity desired. Those skilled in the art readily recognize how such conditions can be varied to vary specificity and selectivity. For example, under highly stringent hybridization conditions only highly complementary nucleic acid sequences hybridize. Preferably, such conditions prevent hybridization of nucleic acids having even one or two mismatches out of 20 contiguous nucleotides.

The term “a reference expression level of genes characteristic of metastatic breast cancer cells” refers to the determined level of gene expression for a breast cancer cell or cell line that is known to possess certain attributes including the ability to extravasate to the lung stroma and exit dormancy to form a secondary tumor site. Examples include the breast cancer cell line AT1 but not cell types that lack one of intravasation, lung homing and metastasis.

Primary breast cancers that express a discrete Coco signature of gene expression are fated to give rise to clinically detectable lung metastases. The data disclosed herein reveal that a multiplex Q-PCR assay or a customized DNA microarray platform can distinguish patients who are destined to develop lung metastases from those who are not. This provides a mechanism for predicting relapse/metastasis to the lung in breast cancer patients, overall survival, and informs physicians decisions with regard to treatment.

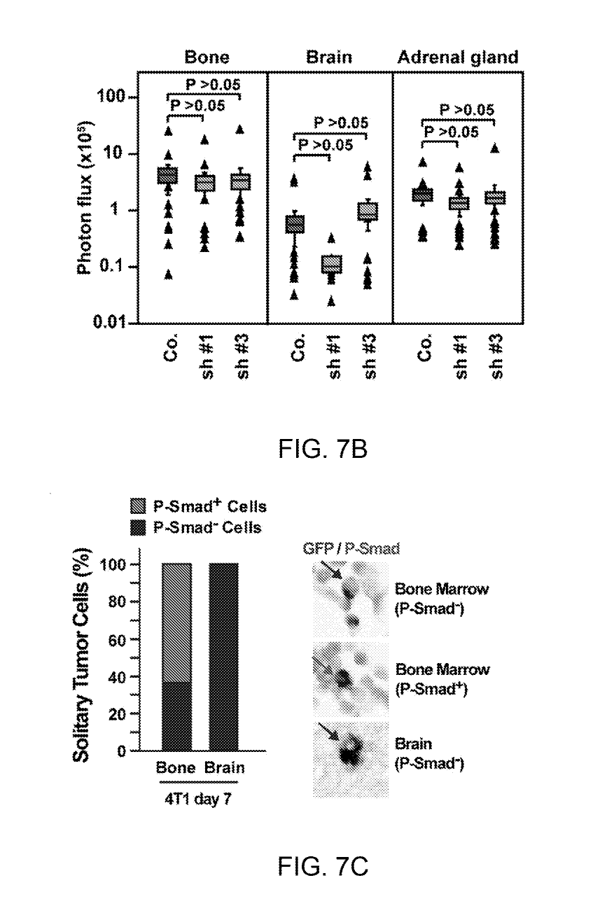

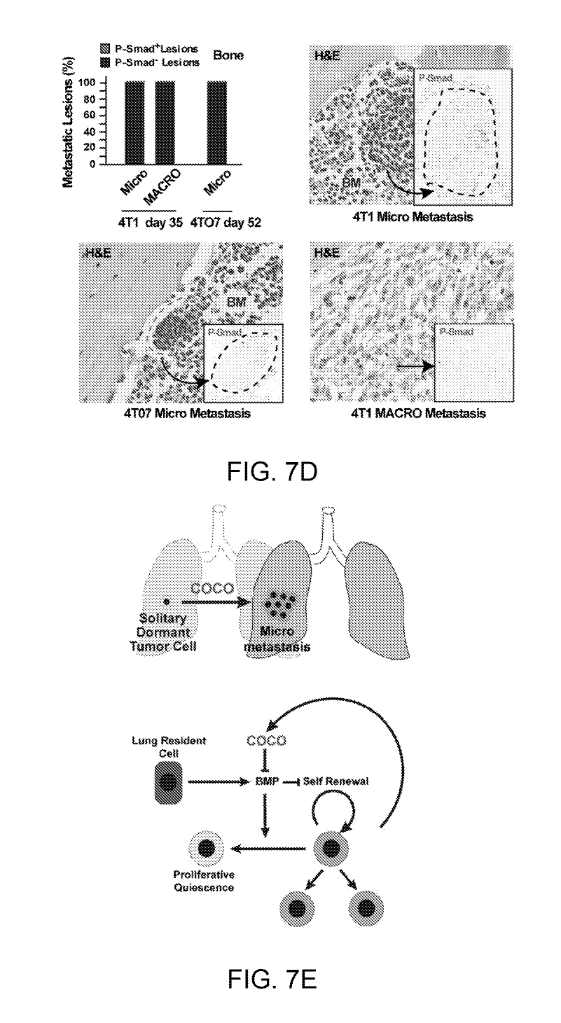

The mechanisms that underlie the reactivation of disseminated breast cancer cells are poorly understood. It has been argued that target organs, such as lung, bone, and brain, represent inhospitable environments for transformed derivatives of epithelial cells of the breast. However, it is unclear to what extent this mismatch arises from tumor cell-intrinsic defects or by barriers imposed by the local microenvironment of the target organ (Nguyen et al., 2009; Weinberg, 2008). We have found that breast cancer cells that have successfully extravasated in the lung parenchyma and survived initial attrition remain dormant for an extended period because stroma-derived BMP proteins limit their ability to proliferate. Production of Coco enables a fraction of these cells to overcome inhibitory BMP signaling and to outgrow into macroscopic metastases. These findings suggest that, although most disseminated tumor cells may possess intrinsic defects that preclude them from surviving or undergoing active proliferation in the lung, those that are fated to give rise to clinical metastases, the metastasis-initiating cells, have to overcome strong anti-metastatic signals which originate in the parenchyma of this organ (FIG. 7).

Several lines of evidence indicate that Coco induces breast cancer cells to exit from dormancy by blocking paracrine BMP signalling. First, whereas both the dormant 4T07 cells and the lung metastatic 4T1 cells are able to produce modest amounts of BMP, the normal cells residing in the mammary gland and lung express significant levels of BMP. Expression of Coco inhibited paracrine BMP signalling in cancer cells, enabling their initial outgrowth in the mammary gland as well as re-activation at lung metastatic sites. Second, virtually all the 4T07 cells that had extravasated in the lung were quiescent and displayed BMP-responsive P-Smads in their nuclei, suggesting that stroma-derived BMP proteins were sufficient to activate BMP signaling in all extravasated tumor cells. Expression of Coco enabled a small fraction of these cells to neutralize stroma-derived BMP proteins, as judged from depletion of C-terminal P-Smads from their nuclei, and to outgrow into macroscopic metastasis. Third, silencing of Coco reinstated the dormant state and thereby suppressed the ability of 4T1 cells to colonize the lung. Fourth, a dominant negative form of the BMP-R and Smad6, which selectively inhibits canonical BMP signaling, replicated the prometastatic effect of Coco in 4T07 cells. Conversely, expression of a constitutively activated BMP receptor suppressed the metastatic ability of 4T1 cells. Finally, further attesting to the generality of the identified mechanism, silencing of Coco suppressed or inhibited the ability of several breast cancer cell lines to colonize the lung. These results provide strong evidence that neutralization of stroma-derived BMP proteins is necessary for metastatic reactivation in the lung.

The lag phase that separates the entry of tumor cells producing Coco into the lung stroma and their outgrowth is consistent with the hypothesis that additional adaptive mechanisms mediate lung colonization. In fact, signals initiated by β1 integrins and mediated by Focal Adhesion Kinase mediate colonization of the lung, seemingly by enabling the initial outgrowth of tumor cells that have established productive interactions with the interstitial matrix of this organ (Shibue and Weinberg, 2009). In addition, recent studies have revealed that the extracellular matrix proteins Tenascin C and Periostin organize niches that nurse outgrowing micrometastases by modulating Notch and Wnt signalling, respectively (Malanchi et al., 2012; Oskarsson et al., 2011). Although these signaling mechanisms have not been specifically linked to exit from solitary tumor cell dormancy, it is possible that they cooperate with Coco to drive this process. Alternatively, they may act following initial outgrowth to foster the expansion of micrometastatic lesions, similar to the role of VCAM-1 in the progression of osteolytic bone lesions (Lu et al., 2011). Our observation that activated β-catenin does not mediate exit from solitary tumor cell dormancy is consistent with the hypothesis that Periostin falls in this latter class of prometastatic entities.

It has been proposed that only a subpopulation of tumor cells—the so called cancer stem cells—possess the extensive self-renewal capability necessary for successful colonization of target organs (Clevers, 2011; Shackleton et al., 2009; Valastyan and Weinberg, 2011). Our results suggest that BMP enforces tumor dormancy by repressing two key cancer stem cell traits: self-renewal in vitro and tumor initiation in vivo. They further suggest that Coco induces exit from dormancy by reversing the ability of BMP to inhibit cancer stem cell function. This model is consistent with previous studies indicating that activation of the BMP pathway inhibits self-renewal and promotes differentiation in pluripotent stem cells (Spagnoli and Hemmati-Brivanlou, 2006) and various adult stem cells (He et al., 2004; Lim et al., 2000). In addition, it is in general agreement with the recent finding that inhibition of BMP signaling contributes to induce and maintain mesenchymal and stem cell states in immortalized human mammary epithelial cells and their Ras-transformed derivatives (Scheel et al., 2011).

Of interest, BMP profoundly inhibited the expression of the transcription factors Nanog, Sox-2, and Oct-4, which comprise the core regulatory circuit that sustains embryonic stem cells (Young, 2011), and of the Hippo transducer Taz, which confers stem cell-related traits on breast cancer cells (Cordenonsi et al., 2011). In contrast, Coco enhanced the expression of these transcriptional regulators by reversing the effect of BMP. Although we have not investigated the interrelationships between these transcriptional regulators, we presume that they are complex, and that each component contributes to sustain a cancer stem cell program of gene expression, underlying the functions necessary for metastatic reactivation. In fact, the transcriptional modules that include as core components Nanog, Sox-2 and Oct-4 as well as Taz/Yap are overexpressed in aggressive and metastatic solid tumors, including breast cancer (Ben-Porath et al., 2008; Wong et al., 2008).

Coco belongs to a large family of secreted BMP antagonists, which play key roles during embryonic development and lineage specification (De Robertis and Kuroda, 2004). The present findings support the model that Coco has a unique ability to induce breast cancer cells to exit from dormancy at lung metastatic sites. In fact, analysis of a panel of human breast cancer cells indicated that Coco was the only BMP inhibitor whose expression correlated with lung metastatic capacity. Furthermore, although the MDA-MB231 cells expressed Chordin-like 1, DAN, and Noggin at levels similar to those of Coco, knock down of Coco was sufficient to block the lung metastatic capacity of these cells. Although all secreted BMP inhibitors function as ligand traps, some have additional functions: Chordin assists in the proteolytic processing and activation of BMP proteins, and Noggin and Sclerostin neutralize each other. Furthermore, individual members differ in oligomerization state, affinity for individual BMP proteins, and ability to bind to the extracellular matrix (Walsh et al., 2010). Therefore, Coco might be a particularly potent mediator of lung colonization because it has a very high affinity for BMP proteins or because it binds to the pericellular matrix and therefore reaches a very high concentration near the cell surface. However, we cannot exclude the possibility that Coco induces metastatic reactivation in the lung also through BMP-independent mechanisms, which remain to be defined.

The observation that Coco promotes colonization of the lung, but not of the bone or brain, reveals the existence of organ-specific barriers to metastatic reactivation. A large fraction of breast cancer cells that infiltrated the bone marrow stroma and virtually all of those that lodged in the brain parenchyma to give rise to metastatic outgrowths did not display nuclear accumulation of BMP-responsive P-Smads, suggesting that they had not been exposed to high levels of bioactive BMP. Thus, although they may face additional barriers, tumor cells that infiltrate the bone or the brain do not need to neutralize locally produced BMP in order to outgrow. Since expression of recombinant BMP or even pre-treatment with BMP blocks the ability of MDA-MB231 cells to colonize the bone after intracardiac injection (Buijs et al., 2007; Buijs et al., 2011), we speculate that breast cancer cells that infiltrate the bone are sensitive to the inhibitory action of BMP. They simply do not need to avert it through production of Coco.

In agreement with its potential additional role in tumor initiation, Coco was not detectable in normal breast tissue but was produced by tumor cells in both lobular and ductal invasive carcinomas. A 14-gene Coco signature predicted relapse to the lung but not to the bone or brain, in agreement with the notion that Coco specifically promotes colonization of the lung. Provocatively, further distillation of the signature led to the identification of 2 genes that maintained an intact ability to predict relapse to the lung and, in fact, participated in this process in a mouse model. Based on these observations, the 14-gene and 2-gene signatures may be used to identify patients with a significant risk to develop lung metastases. In conjunction with similarly developed signatures predicting relapse to other organs, the Coco signatures might enable a more precise characterization of metastatic risk than currently available methodologies. Furthermore, whereas administration of monoclonal antibodies or other biological agents blocking Coco may specifically interfere with lung relapse, BMP receptor agonists might exhibit a broader antimetastatic activity.

Coco emerged from a gain-of-function retroviral cDNA screen in a mouse model of lung metastatic dormancy. The identification of Coco demonstrates that such screens could be used to define the genes that mediate the exit of solitary tumor cells from dormancy in the lung. Furthermore, similar screens using genome-wide shRNA libraries enable the identification of genes that promote dormancy. Finally, both cDNA and shRNA screens can be applied to other mouse models to identify the mechanisms that regulate tumor dormancy at other metastatic sites or to investigate other steps of the metastatic cascade. By affording the advantage of rapid biological validation of single entities that are able to enforce dormancy or mediate exit from it, such gain-of-function genetic screens will lead to the identification of additional potential therapeutic targets for the treatment of metastatic disease.

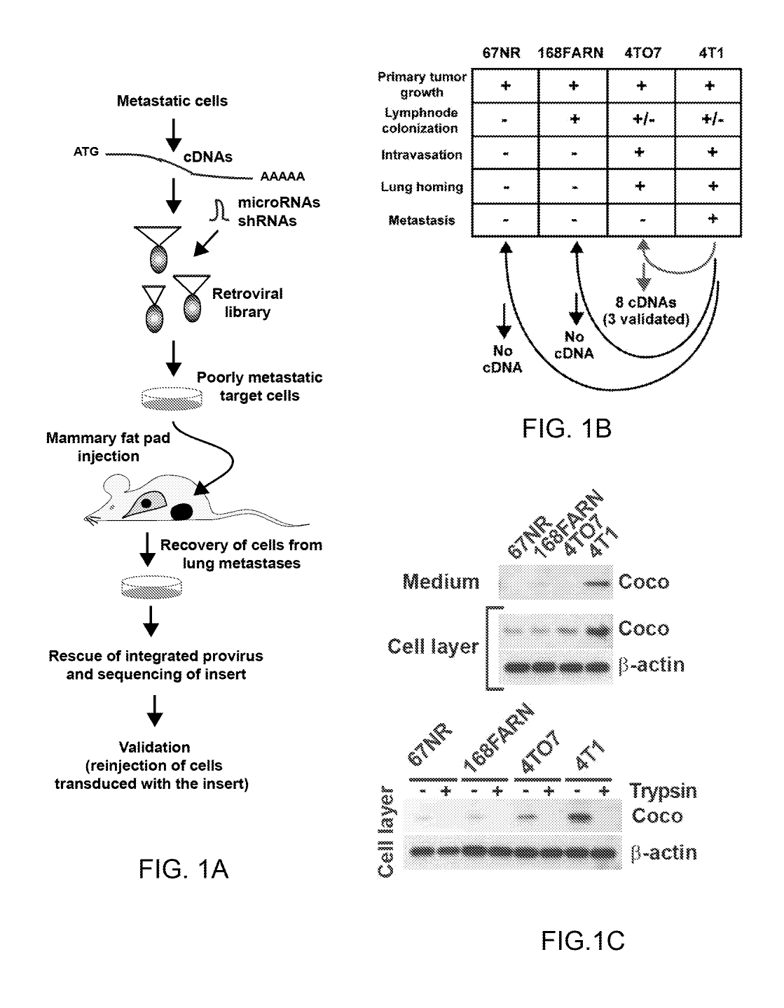

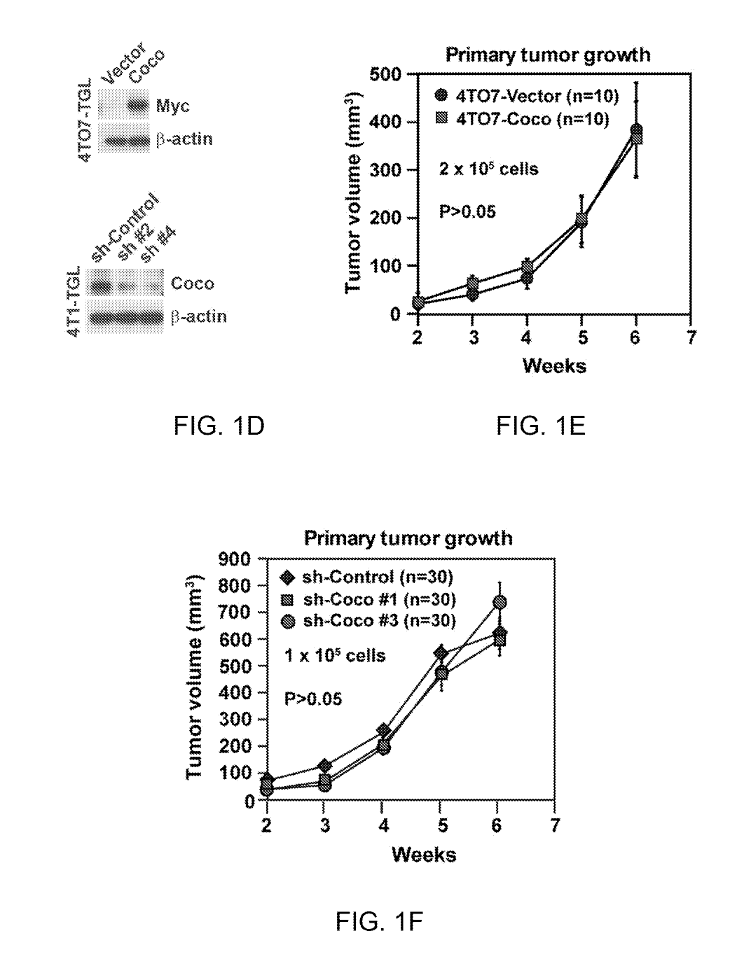

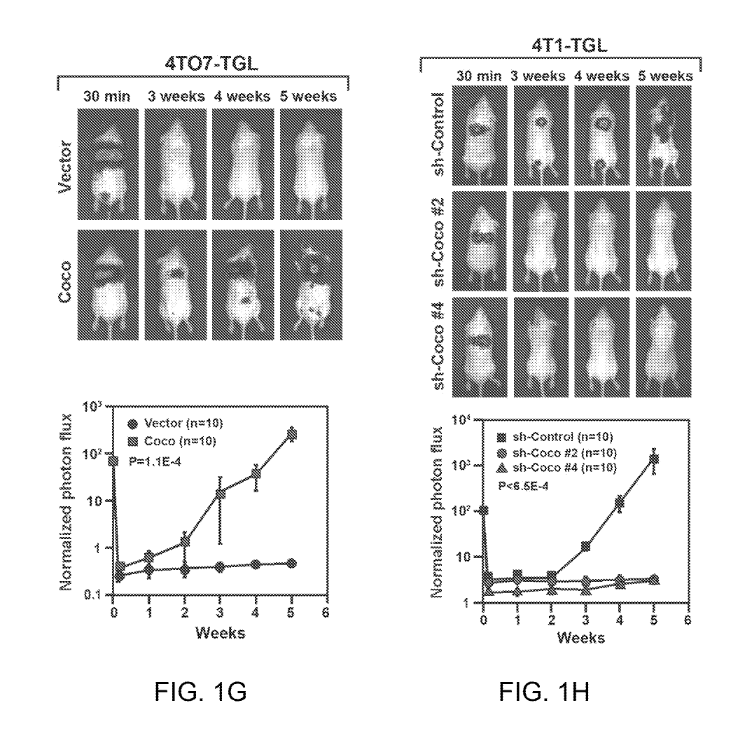

The present invention, therefore, provides a novel discovery platform, a gain-of-function genetic screen that uses the mouse as a filter to identify/isolate genes that mediate metastasis (FIG. 1A). Retroviral libraries consisting of cDNAs, or alternatively, microRNAs or shRNAs from highly metastatic cancer cells, are transduced into poorly metastatic target cells. The transduced cells are then injected into a relevant site, for example, the mammary fat pad of a mouse. After a lag time, the mice are sacrificed and cancer cells that have acquired the ability to colonize a target organ are isolated from individual lesions. Finally, rescue and sequencing of the integrated provirus allows identification of the cDNA, miRNA, or shRNA and confirmation of their pro-metastatic capacity (FIG. 1).

The present invention also provides gene expression profiles and methods of using them to identify the likelihood of recurrence of breast cancer, in particular with metastasis to the lung. In particular, the present application discloses a set of genes, the expression of which in breast cancer cells of a patient correlates with the likelihood of metastasis to the lung. The novel gene signature is based on the observation that particular genes that are differentially expressed in certain breast cancer cells mediate the exit of tumor cells from dormancy, primarily in the lung. Accordingly, the present invention relates to gene expression profiles useful in assessing prognosis and/or predicting the progression of cancer, e.g. breast cancer.

To examine the levels of gene expression of one or more sequences of interest, a biological sample obtained from a patient that is suffering from cancer is typically assayed. A biological sample typically starts with cells from a tumor or cancerous tissue, that has been obtained by biopsy or surgical excision. The cells are then assayed for the presence of one or more gene expression products such as RNA, cDNA, cRNA, protein, etc.

In one embodiment, mRNA from a biological sample is used directly in determining the levels of expression of a group of genes. In another embodiment, RNA is obtained from the biological sample and reverse transcribed to generate a cDNA (complementary DNA) copy using methods well known in the art. In particular embodiments, the cDNA is labeled with a fluorescent label or other detectable label. The cDNA is then hybridized to a substrate containing a plurality of probes of interest. A probe of interest typically hybridizes under stringent hybridization conditions to at least one DNA sequence of a gene signature. In certain embodiments, the plurality of probes are capable of hybridizing to the sequences of at least one of the group of DNA sequences of groups (d)-(f) under the hybridization conditions of 6.times.SSC (0.9 M NaCl, 0.09 M sodium citrate, pH 7.4) at 65.degree. C. The probes may comprise nucleic acids. An example of a nucleic acid is DNA. The term “nucleic acid” refers to deoxyribonucleotides or ribonucleotides and polymers thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, peptide-nucleic acids (PNAs).

In certain cases, the probes will be from about 15 to about 50 base pairs in length. The amount of cDNA hybridization can be measured by assaying for the presence of the detectable label, such as a fluorophore. The quantification of the hybridization signal can be used to generate a score for a particular sequence or set of sequences in the gene signature for a particular patient or plurality of patients.

Included within the scope of the invention are DNA microarrays containing a plurality of sequences that hybridize under stringent hybridization conditions to one or more of the gene sequences in a gene signature. An example of a substrate containing one or more probes of interest is a plurality of DNA probes that are affixed to a substrate. In certain embodiments, the substrate may comprise one or more materials such as gel, nitrocellulose, nylon, quartz, glass, metal, silica based materials, silica, resins, polymers, etc., or combinations thereof. Typically, the DNA probes comprise about 10-50 bp of contiguous DNA. In certain embodiments, the DNA probes are from about 20 to about 50 bp of contiguous DNA. In certain embodiments, the present invention relates to kits which comprising a microarray directions for its use. The kit may comprise a container which comprises one or more microarrays and directions for their use.

The biological sample may also be analyzed for gene expression of one or more genes in a signature using methods that can detect nucleic acids including, but not limited to, PCR (polymerase chain reaction); RT-PCT (reverse transcriptase-polymerase chain reaction); quantitative PCR, etc.

In certain embodiments, the levels of gene expression are measured by detecting the protein expression products of the genes or DNA sequences. The levels of protein products may be measured using methods known in the art including the use of antibodies which specifically bind to a particular protein. These antibodies, including polyclonal or monoclonal antibodies, may be produced using methods that are known in the art. These antibodies may also be coupled to a solid substrate to form an antibody chip or antibody microarray. Antibody or protein microarrays may be made using methods that are known in the art.

Once the levels of gene expression have been measured then a signature score is created. Examples of how to create a signature score are described herein. The signature score is then correlated with a predicted response to cancer treatment. Typically, a Kaplan-Meier curve may be generated to determine if the signature score is associated with a higher or lower survival rate. In particular embodiments, a positive or negative numerical weight may be assigned to a sequence or Unigene ID No. in the creation of a signature score. If the signature score is associated with a lower survival rate, then aggressive cancer treatment may be indicated. If the signature score is associated with a higher survival rate then less aggressive cancer treatment may be indicated.

### EXAMPLES

### Example 1

### Gain of Function for Genes that Mediate the Post-Dissemination Phase of Metastasis

Gain of Function Using cDNA

A gain-of-function genetic screen was designed that uses the mouse as a filter to isolate genes that mediate metastasis (FIG. 1A). In this system, retroviral libraries consisting of cDNAs from highly metastatic cancer cells were transduced into poorly metastatic target cells. The transduced cells were then injected into the mammary fat pad of mice. After a lag time, the mice were sacrificed and cancer cells that had acquired the ability to colonize the lung were isolated from individual lesions. Finally, rescue and sequencing of the integrated provirus allowed identification of the cDNA and confirmation of its pro-metastatic capacity.

This screening strategy was applied to a series of mammary carcinoma cell lines, which were independently derived from a single spontaneous tumor arising in Balb/C mice and appear to be arrested at defined steps of metastasis (FIG. 1B). Upon orthotopic injection in syngeneic mice, 67NR cells give rise to non-invasive primary tumors, 168FARN cells also colonize locoregional lymphnodes but do not gain access to the vasculature, and 4T07 cells enter the vasculature and seemingly home to the lung but do not produce visible metastatic lesions. In contrast, 4T1 cells complete all these steps and produce macroscopic metastases in the lung (Aslakson and Miller, 1992). Interestingly, upon transduction with 4T1 libraries, 67NR or 168FARN cells did not give rise to macroscopic lung metastases in 8 weeks, suggesting that the introduction of a single gene did not enable these cells to penetrate into the bloodstream and acquire all the additional capabilities required for metastatic colonization (FIG. 1B). In contrast, the 4T07 cells infected with the 4T1 libraries produced a total of 8 lung nodules in multiple mice after the same lag time.

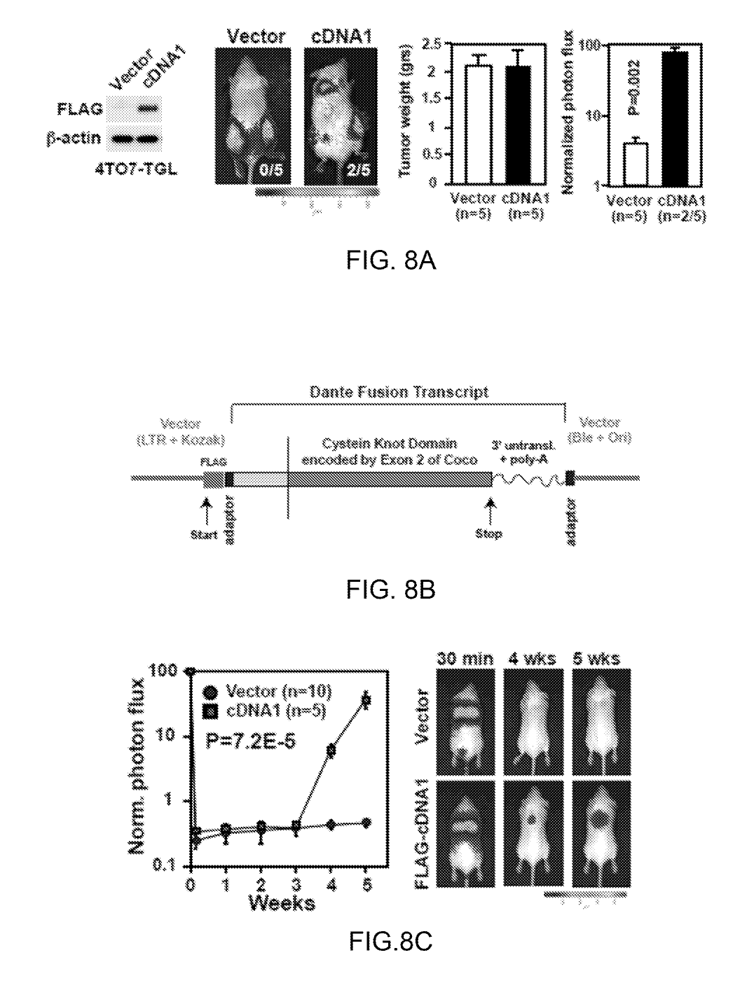

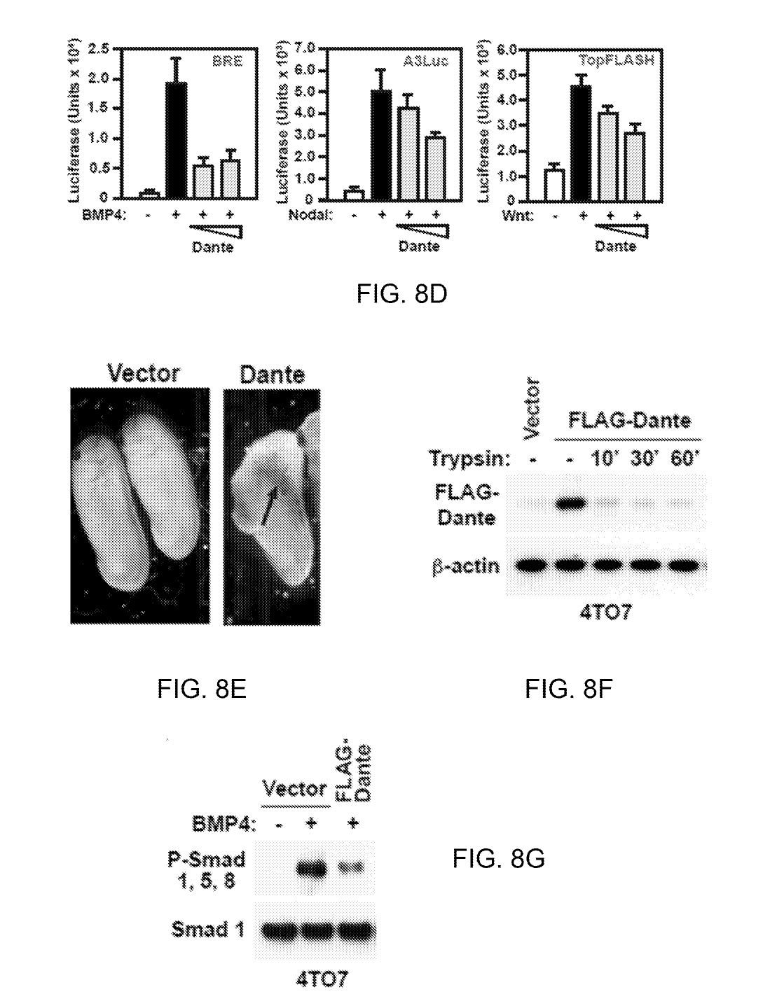

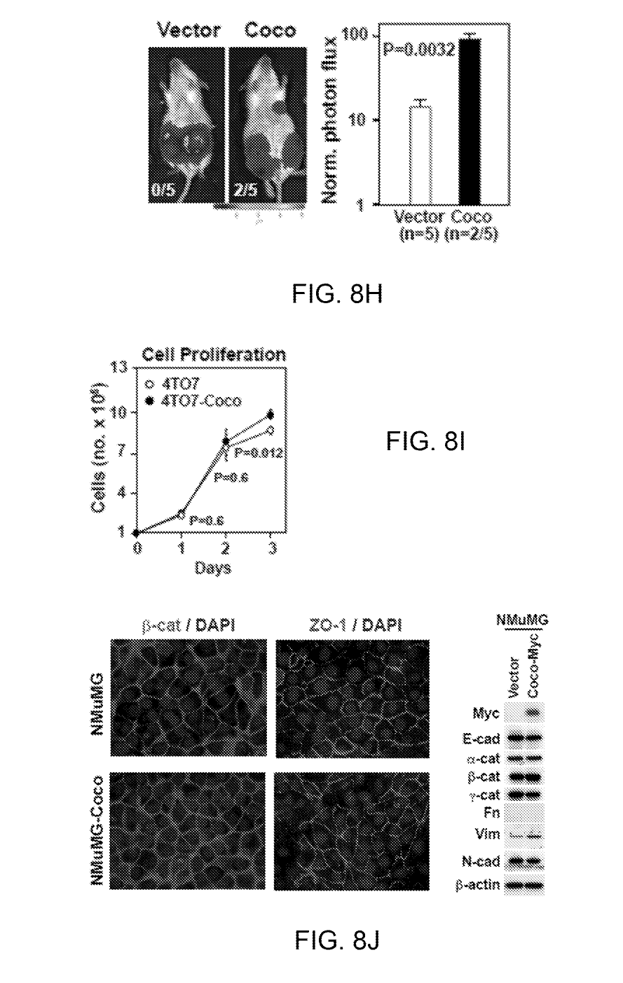

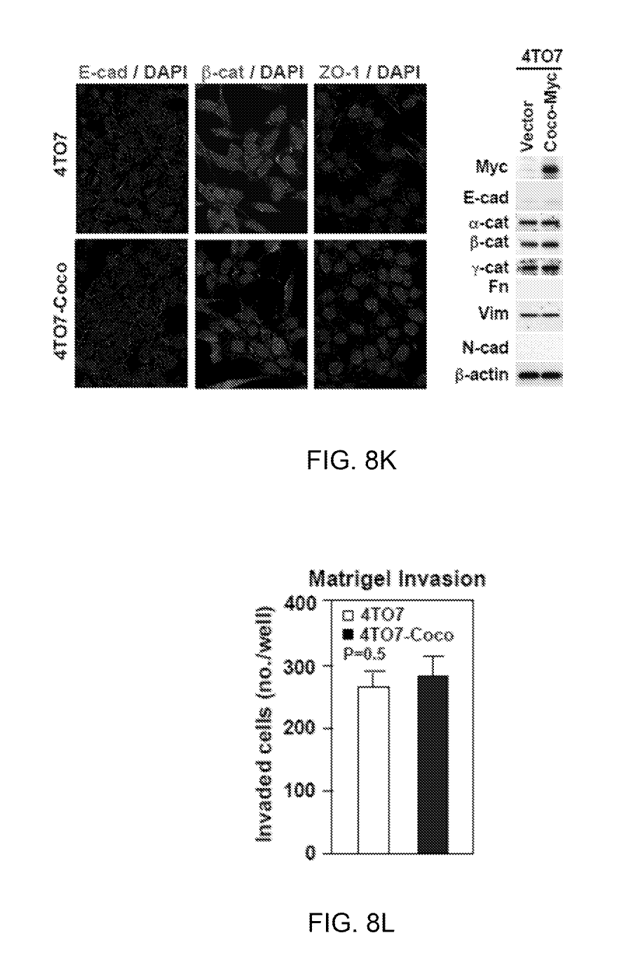

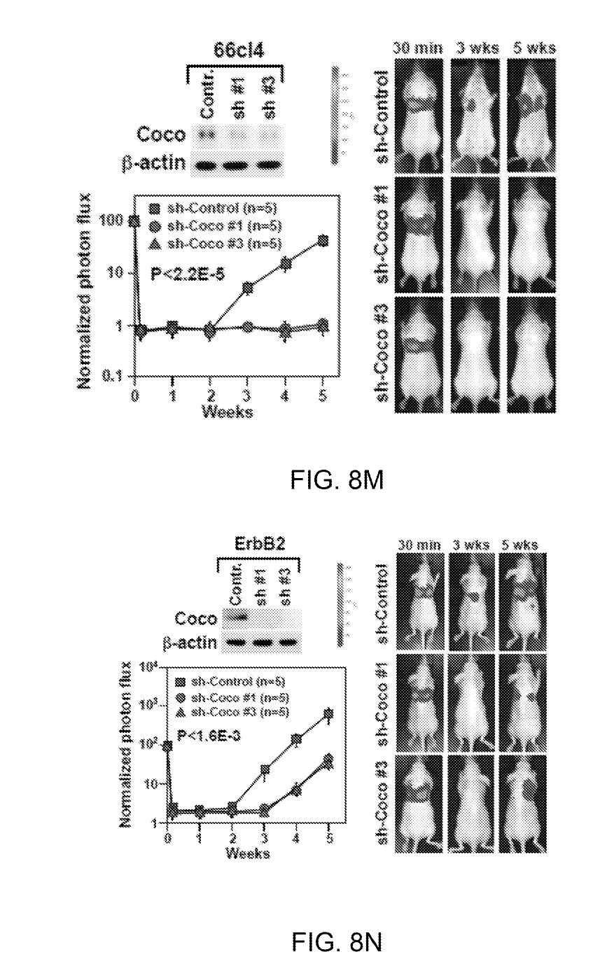

Clonogenic cancer cells were isolated from each of the lesions and the proviruses that had integrated into their genomes were rescued and sequenced. Upon re-introduction in 4T07 cells, 3 of the 8 isolated cDNAs were able to promote lung metastasis without affecting primary tumor growth in a fraction of injected mice (FIG. 8A/S1A). In agreement with the hypothesis that insertional mutagenesis does not contribute to metastasis in this system, 4T07 cells infected with empty vector did not produce macroscopic lung metastases upon injection in 30 mice. These results indicate that retroviral transduction of cDNA libraries into 4T07 cells could be used to isolate cDNAs potentially involved in the homing and outgrowth step of metastasis.

Gain of Function Using miRNA

Prior studies have implicated mir-335, mir-200c, and mir-31 in breast cancer dissemination, although not specifically in reactivation at metastatic sites (35-37). To identify microRNAs (miRNA) able to promote exit from tumor dormancy, a library of mouse microRNAs (˜300) cloned in the retroviral vector pMSCVF was obtained and supplemented with 25 additional microRNAs, which had been identified as upregulated >2-fold in 4T1 cells as compared to 4T07 cells by using microRNA microarray analysis and subsequently cloned (not shown). To identify microRNAs able to promote reactivation in the lung, the library was subdivided into 6 pools and each pool was used to infect 4T07 cells at a low MOI. Each of the 6 populations of transduced 4T07 cells was injected in the tail vein of 5-8 syngeneic mice. Five of the 33 mice developed multiple metastases in their lungs and 2 developed single lesions. Genomic sequencing on tumor cells isolated from these lesions revealed that they harbored 9 integrated microRNA genes. miR-340 and miR-346 were identified in multiple lesions from different mice, suggesting that they are causally implicated in exit from dormancy. The combinations of miR-25 and miR-27a and of miR-138 and miR-223 were identified in a single lesion. To validate these 6 hits, retroviral vectors encoding each of the 9 microRNAs were constructed and used to infect 4T07-TGL cells using single vectors or combinations of vectors. Tail-vein injection followed by bioluminescent imaging indicated that expression of miR-346 or miR-138 efficiently induces lung colonization in the majority of mice, indicating that these microRNAs promote exit from dormancy in the lung. Taken together, these results strongly suggest that the disclosed genetic screen allows the isolation of microRNAs that mediate exit from dormancy.

To determine whether or not miR-138 and miR-346 promote dormancy through an established target or mechanism, the following approach is used to identify the targets that mediate the effect of these microRNAs on dormancy. Sequence analysis with TargetScan and other programs is used in combination with DNA microarray analysis to define potential targets of each microRNA. Additionally, DNA microarray analysis will be performed after transient infection with a high-titer retrovirus encoding the microRNA under analysis. Furthermore, genes suppressed by expression of the microRNA in 4T07 cells are compared with those induced by antagomir-mediated repression of the microRNA in 4T1 cells, focsing on the genes rhat are regulated in a reciprocal manner by more than 2-fold in the two comparisons. Lastly, Q-PCR is used to confirm that the targets are in fact downregulated by the microRNA and luciferase-3′ UTR reporter assays are used to demonstrate that they are bona fide targets.

Gain of Function Using sh-RNA

sh-RNA screens enable the identification of genes, such as Numb, that are involved in enforcing dormancy.

To identify genes that antagonize exit from tumor dormancy, a modified version of the Cancer 1000 shRNA library (39) was screened. The Cancer 1000 library targets ˜1,000 pre-selected ‘cancer genes’, including potential drug targets and many kinases and phosphatases. Depending on initial ‘reconstruction’ studies, this library was subdivided into 6 pools, each containing 288 shRNAs, and was with a seventh pool of 960 additional shRNAs against cancer-related genes (40). To isolate shRNAs that mediate reactivation in the lung, 4T07-TGL cells were transduced with the 7 pools of shRNAs from the modified version of the Cancer 1000 library and the cells expressing each pool were injected into the tail vein of 4-10 syngeneic mice. Metastatic cells were isolated from lung lesions and the potentially pro-metastatic shRNAs that they harbored were sequenced and identified. The screen was then repeated. The hits were prioritized based on the observed frequency of isolation.

The top 20 hits from the Cancer 1000 shRNA Screen, which yielded a total of 51 hits are shown in Table 1.

TABLE 1
Top 20 Hits from the Cancer 1000 shRNA Screen

| | | Origin | |
|---|---|---|---|
| | Lesions | Pools | Mice |
| Identified Genes | (#) | (#) | (#) |
| Cart 1 (Cartilage homeo protein 1/Alx1:ALX | 9 | 5 | 6 |
| homeobox protein 1) | | | |
| Pcna (Proliferating cell nuclear antigen) | 6 | 3 | 5 |
| Cd3e (Mouse CD3 antigen epsilon | 4 | 1 | 1 |
| polypeptide) | | | |
| Cd3g (Mouse CD3 antigen gamma (T3- | 4 | 1 | 1 |
| gamma)) | | | |
| Nek4 (NIMA (never in mitosis gene-a)- | 4 | 3 | 3 |
| related expressed kinase 4) | | | |
| Numb (Numb gene homolog (Drosophila)) | 4 | 3 | 4 |
| Itga3 (Integrin alpha 3) | 3 | 3 | 3 |
| Pdcd5 (Programmed cell death 5) | 3 | 1 | 1 |
| Smyd5 (Mouse SET and MYND domain | 3 | 3 | 3 |
| containing 5) | | | |
| Smurf2 (SMAD specific E3 ubiquitin protein | 3 | 3 | 3 |
| ligase 2) | | | |
| Erbb4 (v-erb-a erythroblastic leukemia viral | 2 | 2 | 2 |
| oncogene homolog 4) | | | |
| Fgf2 (Fibroblast growth factor 2) | 2 | 2 | 2 |
| Gpr34 (G protein-coupled receptor 34) | 2 | 2 | 2 |
| IL2 (Mouse Interleukin 2) | 2 | 1 | 2 |
| Lyn (Lyn Yamaguchi sarcoma viral (v-yes-1) | 2 | 1 | 1 |
| oncogene homolog) | | | |
| March5 (membrane associated ring finger | 2 | 2 | 2 |
| (C3HC4) 5) | | | |
| Sfrs6 (Splicing factor, arginine/serine-rich 6) | 2 | 2 | 2 |
| Shbg (Sex hormone binding globulin) | 2 | 1 | 1 |
| Src (Rous sarcoma oncogene) | 2 | 1 | 2 |
| Yes1 (Yamaguchi sarcoma viral (v-yes-1) | 2 | 2 | 2 |
| oncogene homolog) | | | |

As shown in Table 1, shRNAs targeting 3 genes were recovered in both rounds of screening, indicating that these genes must be inactivated for exit from dormancy. The genes targeted by these shRNAs comprise the Notch inhibitor Numb, which is involved in asymmetric stem cell division (41), the uncharacterized gene Smyd5, and the Smad E3 ubiquitin ligase Smurf2, which opposes TGF-β signaling (42) (Table I). This latter finding is in agreement with the ability of TGF-β to induce a cancer stem cell phenotype and promote metastatic capability (2,3).

Alternative methods for identifying microRNA targets include crosslinking the microRNAs to their target mRNAs, doing a pull down, amplifying by PCR and finally sequencing the target mRNA. (see Chi et al., Nature 460(7254)479-486)

### Example 2

### Secreted TGF-β Ligand Inhibitor Coco Promotes Lung Colonization

One of the 3 pro-metastatic cDNAs identified, herein referred to as cDNA1, which encodes an N-terminally truncated but potentially active version of Coco, a secreted inhibitor of TGF-β ligands was chosen for further evaluation. The same transcript had been isolated during studies on mouse development and referred to as Dante (Pearce et al. 1999) (FIG. 8B/S1B). Bioluminescent imaging indicated that expression of this cDNA enables 4T07 cancer cells injected in the tail vein to metastasize to the lung, indicating that this cDNA promotes the homing and outgrowth step of metastasis (FIG. 8C/S1C). Experiments in Xenopus laevis have shown that Coco binds directly to BMP and Nodal proteins blocking their ability to bind to their cognate receptors (Bell 2003). In addition, expression of Coco interferes with Wnt signaling during early Xenopus embryogenesis. Reporter assays indicated that expression of cDNA1, which comprise the cysteine knot domain of Coco, inhibits BMP signaling and, to a much smaller extent, Nodal and Wnt signaling in Xenopus embryos (FIG. 8D/S1D), in agreement with the model that the cystein knot domain is sufficient for antagonism of BMP proteins. In addition, albeit of different quality, expression of cDNA1 induced axis duplication (FIG. 8E/S1E), similarly to Coco (Bell 2003). Finally, this cDNA directed expression of a secreted protein in 4T07 cells (FIG. 8G/S1G) and expression of this truncated protein interfered with BMP-induced phosphorylation of Smad 1, 5, and 8 in these cells (FIG. 8G/S1G). These results indicate that cDNA1 encodes a truncated but biologically active form of Coco and suggest that expression of Coco promotes metastasis.

To examine if Coco promotes metastasis, Coco expression in the mammary carcinoma metastatic progression series was assessed. Immunoblotting indicated that the 67NR, 168FARN, and 4T07 cells express low levels of Coco, whereas the highly metastatic 4T1 cells express high levels of this inhibitor (FIG. 1C, top). Interestingly, a large fraction of Coco remained associated with the cell layer rather than diffusing into the medium (FIG. 1C, top). Treatment of intact, live cells with modest amounts of highly purified trypsin led to the disappearance of Coco from the cell layer (FIG. 1C, bottom), indicating that this inhibitor associates with the pericellular matrix and might therefore accumulate at high concentrations near the cell surface.

To study the effect of Coco on tumor progression, we expressed full-length Coco in 4T07 cells and silenced Coco in 4T1 cells (FIG. 1D). As anticipated, expression of Coco enabled the 4T07 cells to colonize the lung after orthotopic injection at a rate similar to that of cDNA1 (Figure S1H; compare to Figure S1A). Interestingly, expression of Coco did not affect the ability of these cells to proliferate in culture (Figure S1I) or to grow as primary tumors upon injection of 2×105 cells in the mammary fat pad (FIG. 1E). Conversely, silencing of Coco did not inhibit the capacity of 4T1 cells to produce tumors when 1×105 cells were injected at the orthotopic site (FIG. 1F). Furthermore, expression of Coco did not destabilize the adherents or tight junctions or promote completion of an EMT program in normal mammary epithelial cells and 4T07 cells (Figures S1J and S1K). Finally, expression of Coco did not enhance the ability of 4T07 cells to invade through Matrigel in vitro (FIG. 8L/S1L). These results suggest that Coco does not affect primary tumor growth or the general ability of tumor cells to invade through tissue boundaries. However, expression of Coco enabled the 4T07 cells to metastasize efficiently to the lung upon injection in the tail vein (FIG. 1G). Conversely, silencing of Coco using two distinct shRNAs suppressed the ability of the highly metastatic 4T1 cells to colonize the lung in the same assay (FIG. 1H). These results suggest that expression of Coco is necessary and sufficient to promote the homing and outgrowth step of metastasis.

To examine if Coco enables other mouse mammary carcinoma cells to colonize the lung, we silenced its expression in the 66cl4 cells, which were derived from the same spontaneous tumor as the 4T1 cells but are less aggressive (Aslakson and Miller, 1992), and in primary mammary tumor cells isolated from MMTV-Neu(YD) mice, which represents a model for metastatic HER2+ breast cancer (Dankort et al., 2001; Guo et al., 2006). Immunoblotting indicated that the 66cl4 cells express Coco at levels similar to those in 4T1 cells, whereas the ErbB2-transformed cells express somewhat lower levels of Coco (not shown). Silencing of Coco completely abrogated the lung colonization capacity of 66cl4 cells (FIG. 8M/S1M) and inhibited that of ErbB2-transformed mammary tumor cells by a very large extent (>90% inhibition at 5 weeks) (FIG. 8N/S1N), pointing to a general requirement for Coco during lung colonization.

### Example 3

### Coco Induces Tumor cells to Exit from Dormancy at Lung Metastatic Sites

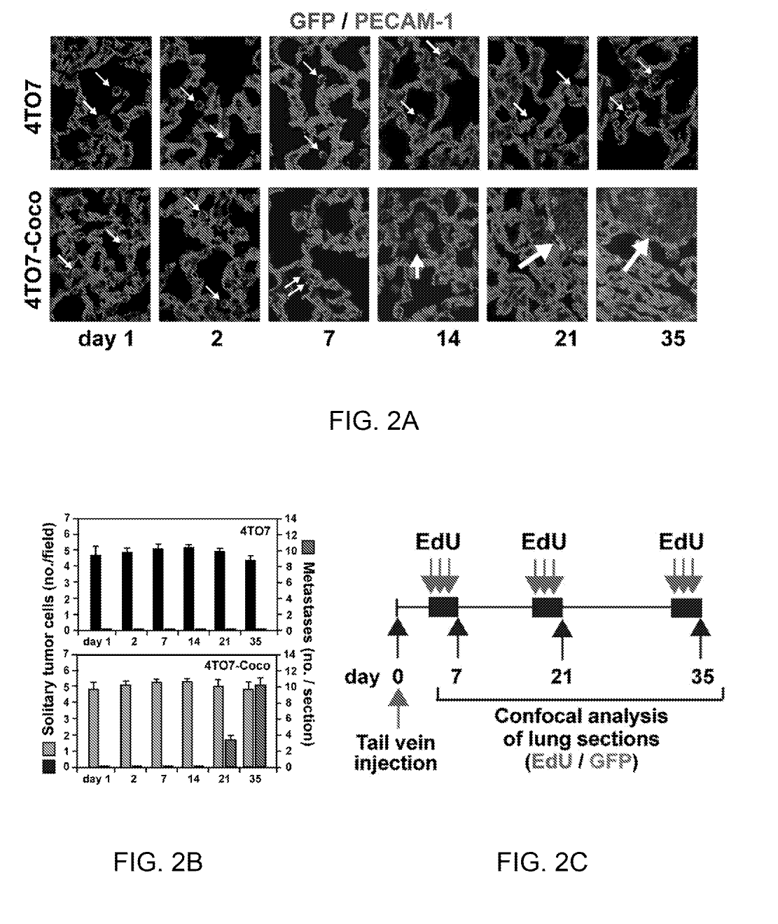

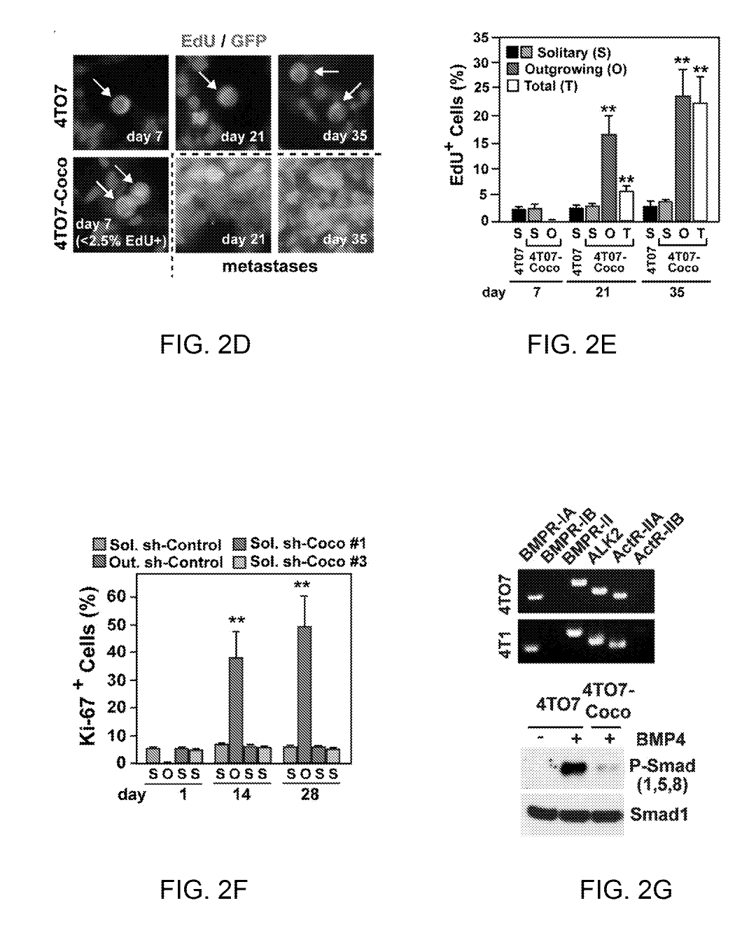

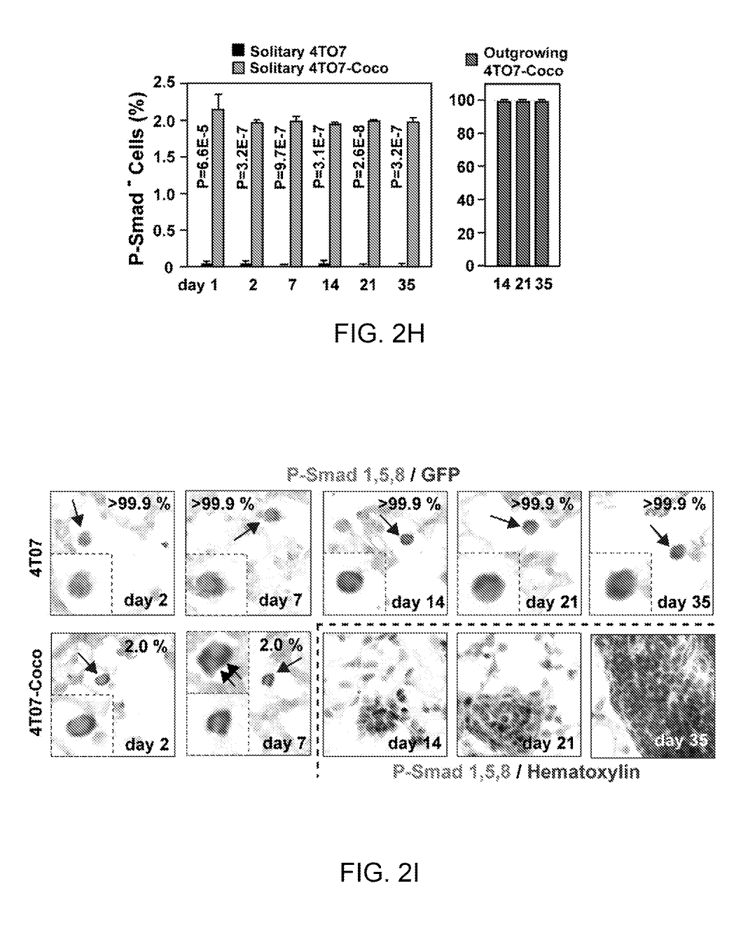

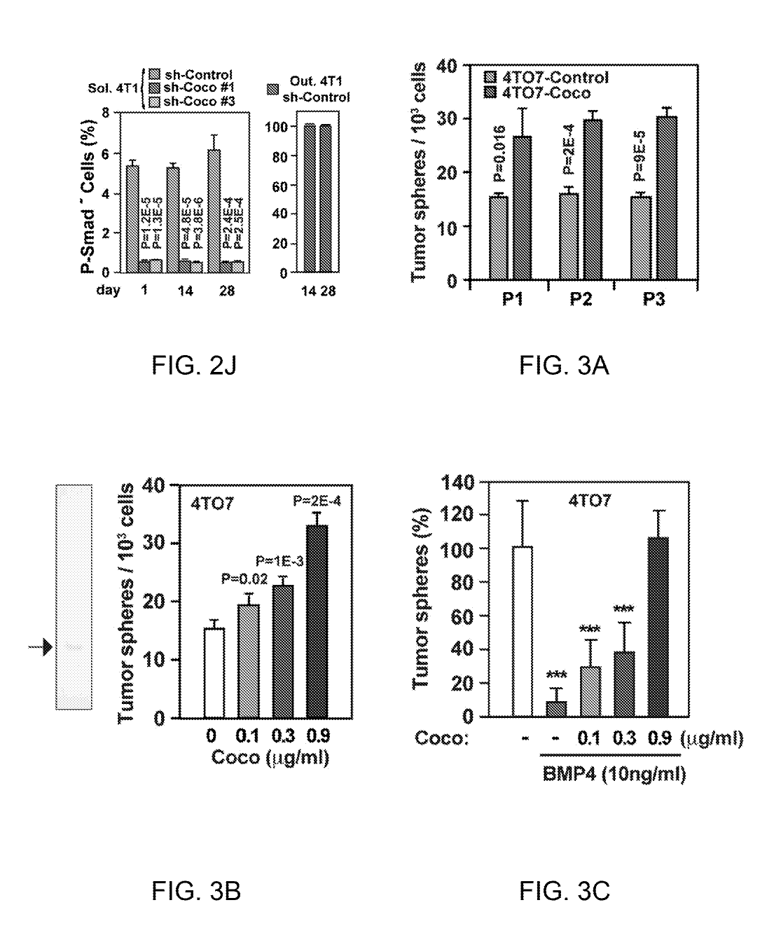

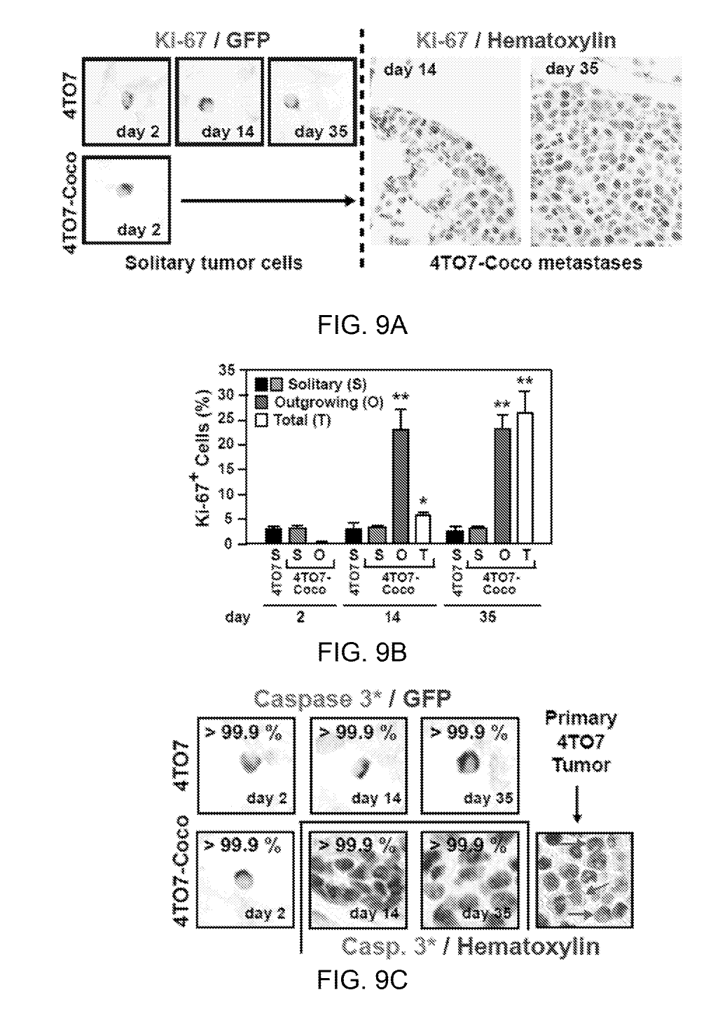

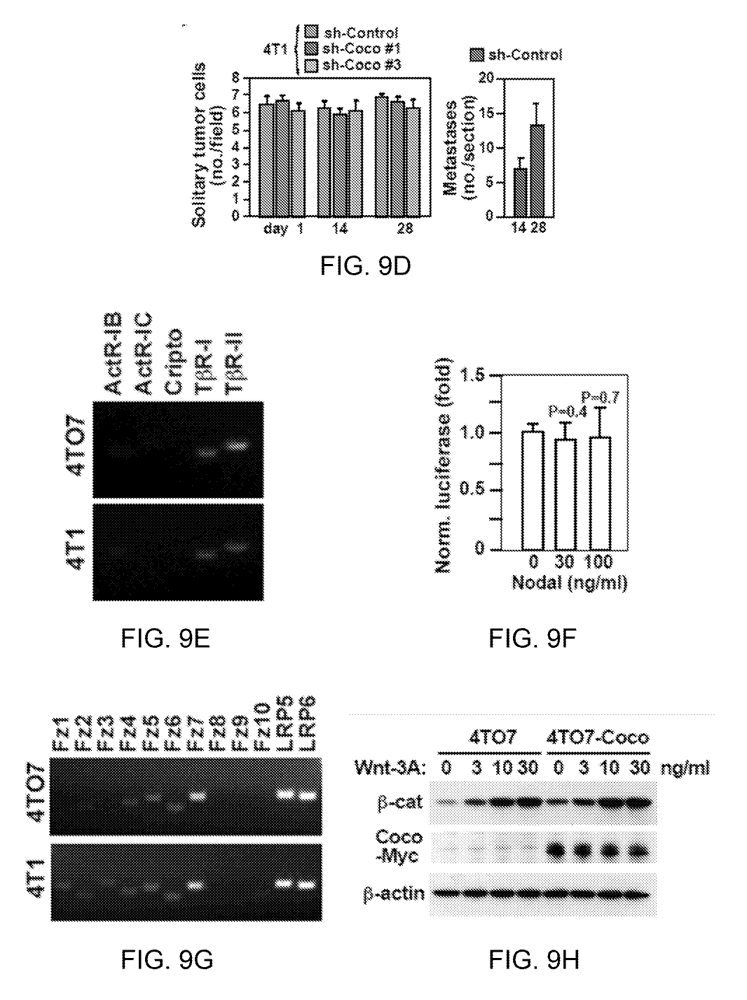

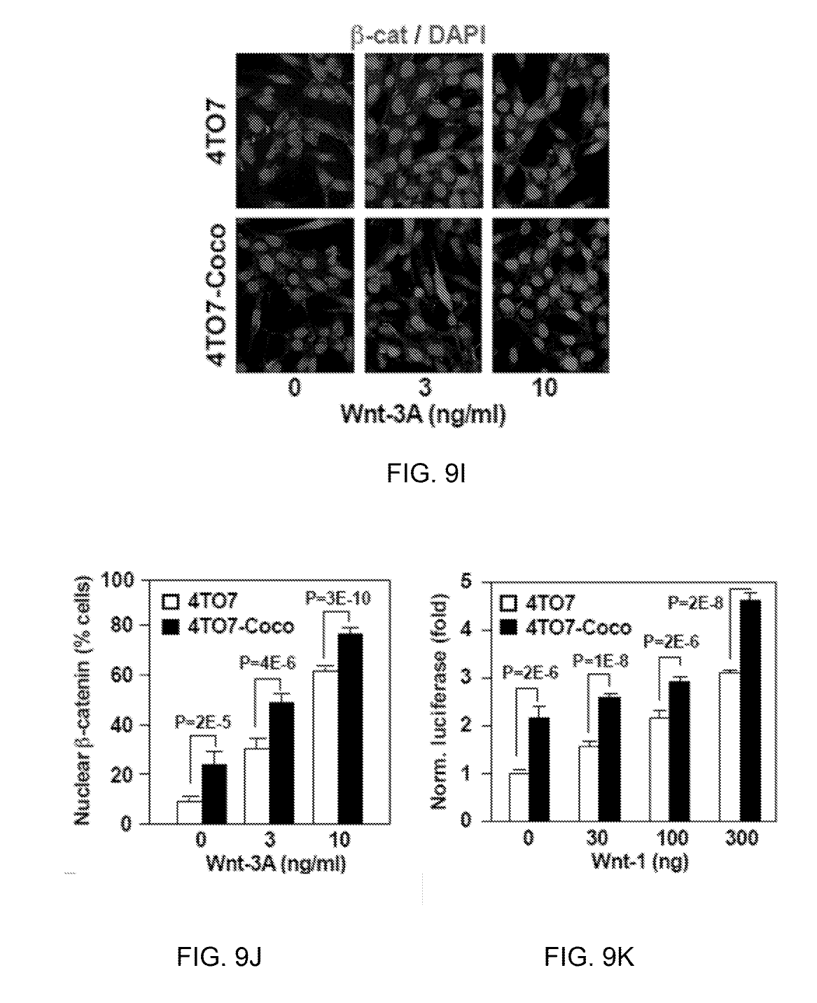

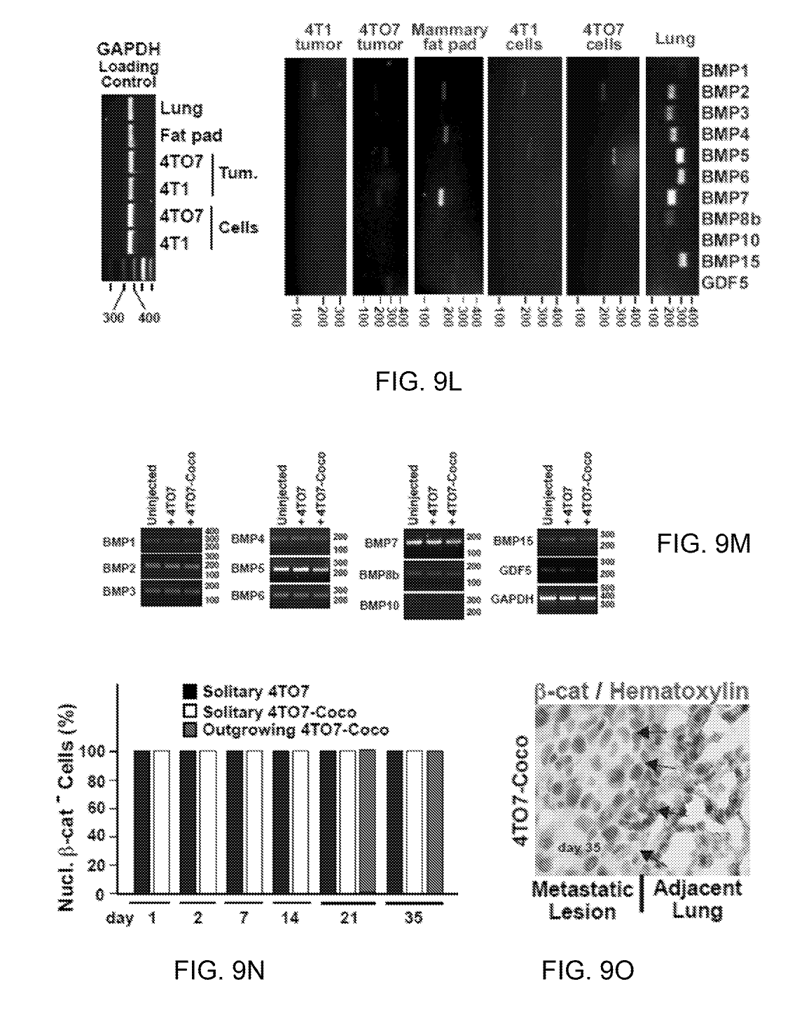

Confocal imaging of lung sections revealed that the 4T07 cells extravasate efficiently in the stroma of the lung within 1 day after injection in the tail vein (FIGS. 2A and 2B). Expression of Coco did not enhance the ability of 4T07 cells to infiltrate the lung. However, whereas control 4T07 cells remained solitary and seemingly quiescent in the stroma of this organ, a small fraction of those expressing Coco started to proliferate from around day 14 and gave rise to metastatic outgrowths (FIGS. 2A and 2B). Most of the lesions detected at day 21 were relatively small and had not yet undergone neoangiogenesis, but those present at day 35 were large and vascularized (FIG. 2A). While the number of solitary tumor cells was comparable to that of outgrowing tumor cells within micrometastases at day 21 (737+18 solitary 4T07; 762+70 solitary 4T07-Coco; 707+22 outgrowing 4T07-Coco per lung section), the solitary tumor cells were far outnumbered by tumor cells within macro-metastases at day 35 (648+40 solitary 4T07; 732+74 solitary 4T07-Coco; 6979+122 outgrowing 4T07-Coco per lung section). Staining with antibodies to Ki67, which accumulates in the S, G2, and M phases of the cell cycle, suggested that a large majority of the solitary 4T07 cells or 4T07 expressing Coco were not actively cycling (>97%). In contrast, the micrometastatic lesions and the large metastases arising from 4T07 cells expressing Coco contained a large fraction of actively proliferating cells (FIGS. 8A/S2A and 8B/S2B). As it could be anticipated from their persistence in the lung and nuclear morphology, anti-cleaved caspase 3 staining indicated that the solitary 4T07 cells and 4T07 cells expressing were not apoptotic at any time point examined (up to 35 days; FIG. 9C).

To confirm that the solitary tumor cells that had extravasated in the lung were quiescent, we performed 5-ethynyl-2′-deoxyuridine (EdU) incorporation experiments as outlined in FIG. 2C. Mice were administered EdU over 3-days periods ending at day 7, day 21 and day 35 following tail-vein injection of 4T07 cells expressing Coco or not. Lung sections were subjected to click chemistry to conjugate the EdU moieties incorporated in DNA to fluorescent azide as well as to anti-GFP staining. Confocal imaging indicated that more than 95% of solitary 4T07 or 4T07-Coco cells did not enter into or traverse the S-phase over each of the 3-days EdU incorporation periods. In contrast, a large fraction of 4T07 cells expressing Coco within outgrowing micrometastic lesions and overt metastases transited through the S phase over the same times (FIGS. 2D and 2E). Taken together, these observations indicated that the 4T07 cells undergo a protracted period of solitary tumor dormancy upon extravasating into the lung stroma. Expression of Coco enabled a fraction of these cells to exit from quiescence and to give rise to metastatic outgrowths.

To examine if silencing of Coco suppresses lung colonization by inducing dormancy, we compared the number of solitary tumor cells in the lungs of mice injected with control and Coco-silenced 4T1 cells at various times after tail-vein injection. As anticipated, silencing of Coco did not inhibit extravasation or decrease the number of solitary tumor cells present on lung sections at day 14 and 28 (FIG. 9D). Furthermore, whereas a fraction of control 4T1 cells initiated proliferation to give rise to metastatic outgrowths, virtually all Coco-silenced cells remained quiescent (FIGS. 2F and 9D). These results suggest that depletion of Coco suppresses lung colonization by preventing the reactivation of solitary tumor cells that have infiltrated this organ.

### Example 4

### Coco Inhibits BMP Signaling in Solitary Tumor Cells Fated to Produce Metastatic Outgrowths

To define the molecular mechanism through which Coco promotes metastatic re-activation, we examined its ability to regulate BMP, Nodal, and Wnt signaling in mammary carcinoma cells. Semiquantitative RT-PCR assays indicated that the 4T07 and 4T1 cells express several type I and type II BMP receptors (FIG. 2G, top), but they do not express nodal receptors or Cripto (FIG. 9E). Accordingly, immunoblotting with antibodies to C-terminal phosphorylated Smad 1, 5, 8 indicated that BMP4 induces robust activation of BMP-responsive Smad proteins in the 4T07 cells and that Coco reverses this process (FIG. 2G, bottom). In contrast, as anticipated from the absence of appropriate membrane receptors, the 4T07 cells did not respond to Nodal in reporter assays (FIG. 9F).

Since Coco can inhibit also Wnt signaling in Xenopus embryos (Bell et al., 2003), Coco's affects on Wnt signaling in 4T07 cells was examined. RT-PCR assays indicated that both 4T07 and 4T1 cells express significant levels of Frizzled7 and LRP5 and 6 (FIG. 9G). Interestingly, expression of Coco did not inhibit but partially enhanced Wnt-induced stabilization and nuclear translocation of β-catenin in 4T07 cells (FIGS. 9H-J). In addition, Coco enhanced Wnt-induced activation of the Wnt-responsive TopFLASH reporter in these cells (FIG. 9K). These results indicate that Coco suppresses BMP signaling but partially enhances Wnt signaling. Genetic studies have shown that the BMP-RIA stabilizes PTEN and thereby inhibit AKT-dependent activation of β-catenin in intestinal and hair follicle stem cells (He et al., 2004; Kobielak et al., 2007). This raises the possibility that Coco reduces the threshold for the propagation of Wnt signaling by alleviating the intracellular inhibitory crosstalk that BMP receptors exert on β-catenin.

To examine the potential contribution of inhibition of BMP signaling to tumor dormancy, sections of lungs infiltrated by GFP-tagged 4T07 cells expressing Coco or not were stained with antibodies to P-Smad 1, 5, 8 and to GFP. Virtually all the solitary, dormant 4T07 cells displayed strong nuclear accumulation of P-Smad proteins (>99%; FIGS. 2H and 2I), suggesting that BMP signaling was robustly activated in these cells. Semiquantitative RT-PCR indicated that the 4T07 and 4T1 cells express low levels of BMP proteins, whereas the cellular elements of the normal lung express significant levels of several BMP proteins, in particular BMP5 and 7 (FIG. 9L). Metastatic seeding of the lung by control 4T07 cells expressing Coco or not did not lead to detectable changes in the expression of BMP proteins (FIG. 9M). In general agreement with these results, prior studies have indicated that the mesenchymal compartment of developing lung expresses high levels of BMP4 and BMP5, whereas the epithelial compartment expresses BMP7 and lower levels of BMP4. Whereas BMP4 is downregulated after its inductive action is no longer required, BMP5 and BMP7 remain elevated (Bellusci et al., 1996; Danesh et al., 2009; King et al., 1994). It was inferred that the 4T07 cells underwent protracted proliferative quiescence in response to BMP proteins that had been produced by both epithelial and mesenchymal cells and deposited in the stroma between alveoli.

Intriguingly, a small fraction (approximately 2%) of the dormant tumor cells present in the lungs of mice injected with GFP-tagged 4T07 cells expressing Coco displayed no nuclear accumulation of P-Smad 1, 5, 8 (FIGS. 2H and 2I), suggesting that microenvironmental fluctuations in the rate of accumulation of Coco in the pericellular matrix or availability of BMP proteins limited inhibition of BMP signaling to a subpopulation of solitary tumor cells transduced with Coco. In agreement with the hypothesis that these rare cells were fated to produce metastatic outgrowths, both the incipient lesions and the macrometastases displayed no nuclear accumulation of BMP-responsive P-Smad proteins (FIGS. 2H and 2I).

In a reciprocal set of experiments, we evaluated if silencing of Coco induced reactivation of Smad signaling in solitary 4T1 cells that would have otherwise been destined to outgrow into metastatic lesions. Analysis of lung sections from mice injected with control 4T1 cells indicated that a small but sizeable fraction of solitary tumor cells (about 5%) and all tumor cells within outgrowing metastases did not display nuclear accumulation of BMP-responsive P-Smad proteins, suggesting that the metastases arise from P-Smad-negative cells (FIG. 2J). This subpopulation of P-Smad-negative cells was not detected in the lungs of mice injected with Coco-silenced 4T1 cells. In fact, more than 99.3% of the dormant Coco-silenced 4T1 cells displayed strong nuclear accumulation of BMP-induced P-Smad proteins (FIG. 2J). Taken together, these findings suggest that Coco promotes exit from dormancy by alleviating the ability of stromal BMP to enforce a dormant state in extravasated tumor cells.

To examine if activation of β-catenin contributed to tumor dormancy, lung sections from mice injected with GFP-tagged 4T07 cells expressing Coco or not were stained with antibodies to β-catenin and to GFP. Virtually all (>99.9%) the solitary tumor cells present in both samples as well as the metastatic lesions derived from those expressing Coco had very low levels of β-catenin and did not exhibit a detectable accumulation of the protein in the nucleus or at cell junctions (FIGS. 9N and 9O), suggesting that activation of β-catenin does not contribute to exit from dormancy. Taken together, these findings suggest that Coco promotes exit from dormancy by inhibiting BMP signalling but not by activating β-catenin signalling.

### Example 5

### Coco Promotes Tumor Sphere Formation In Vitro

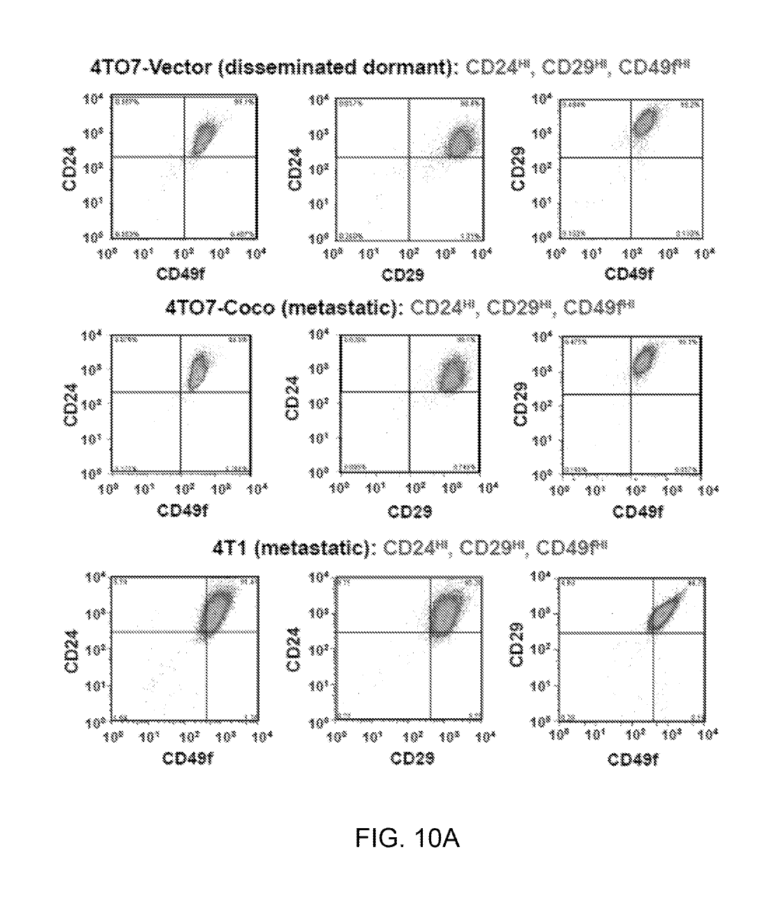

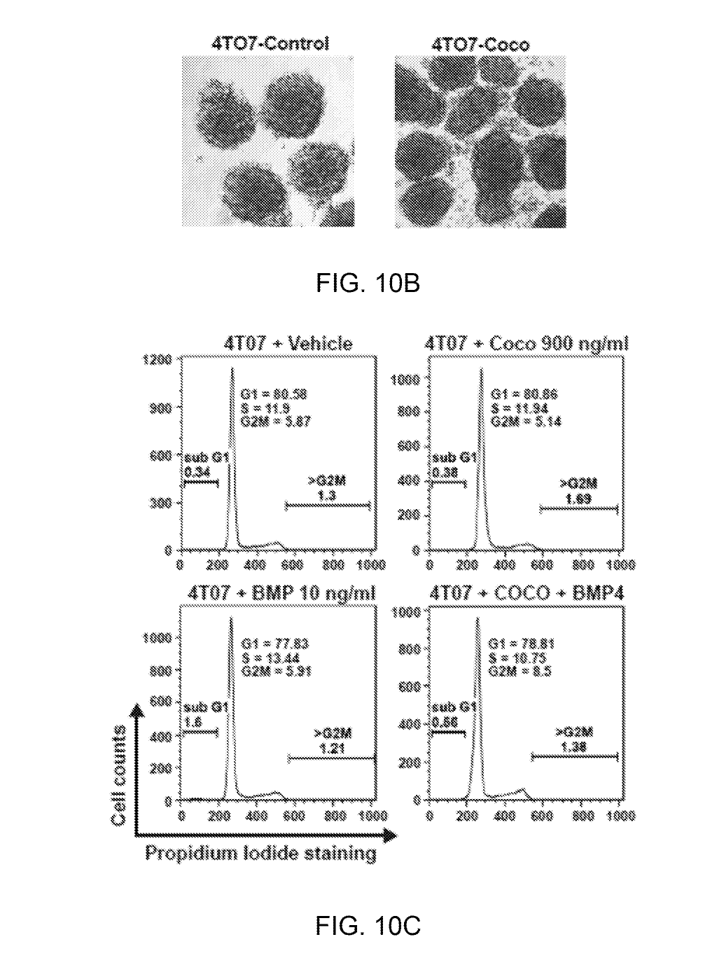

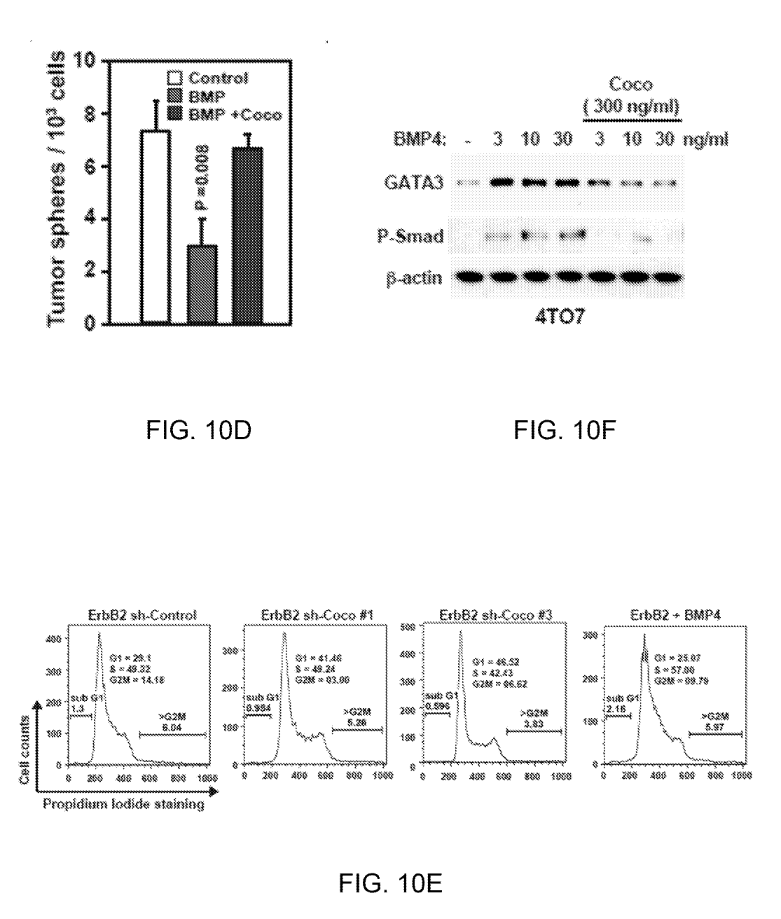

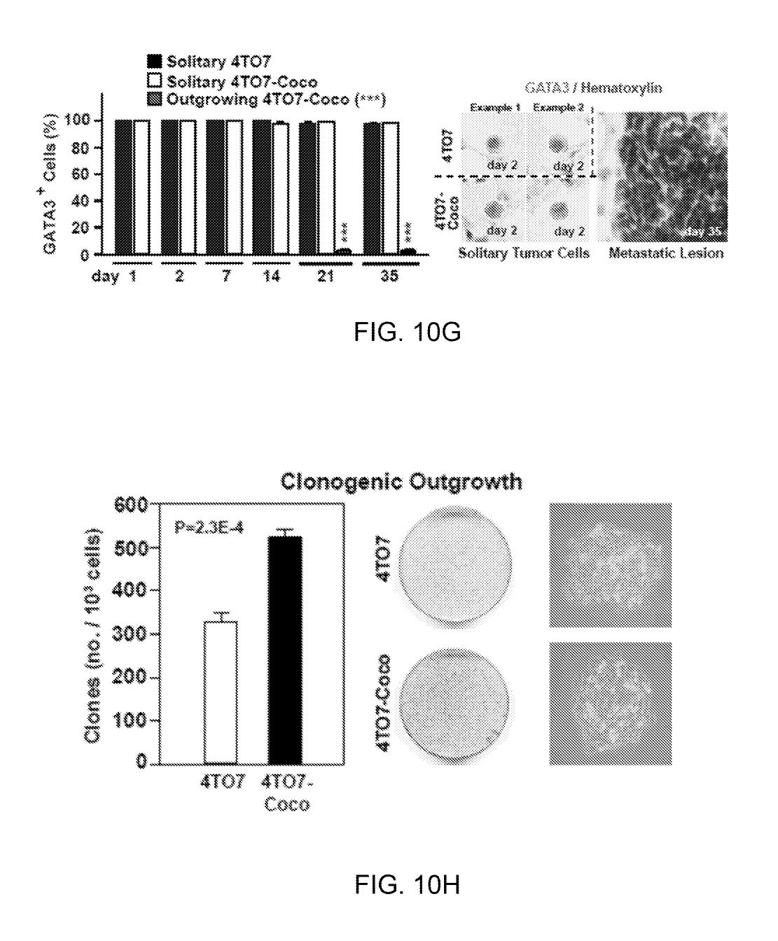

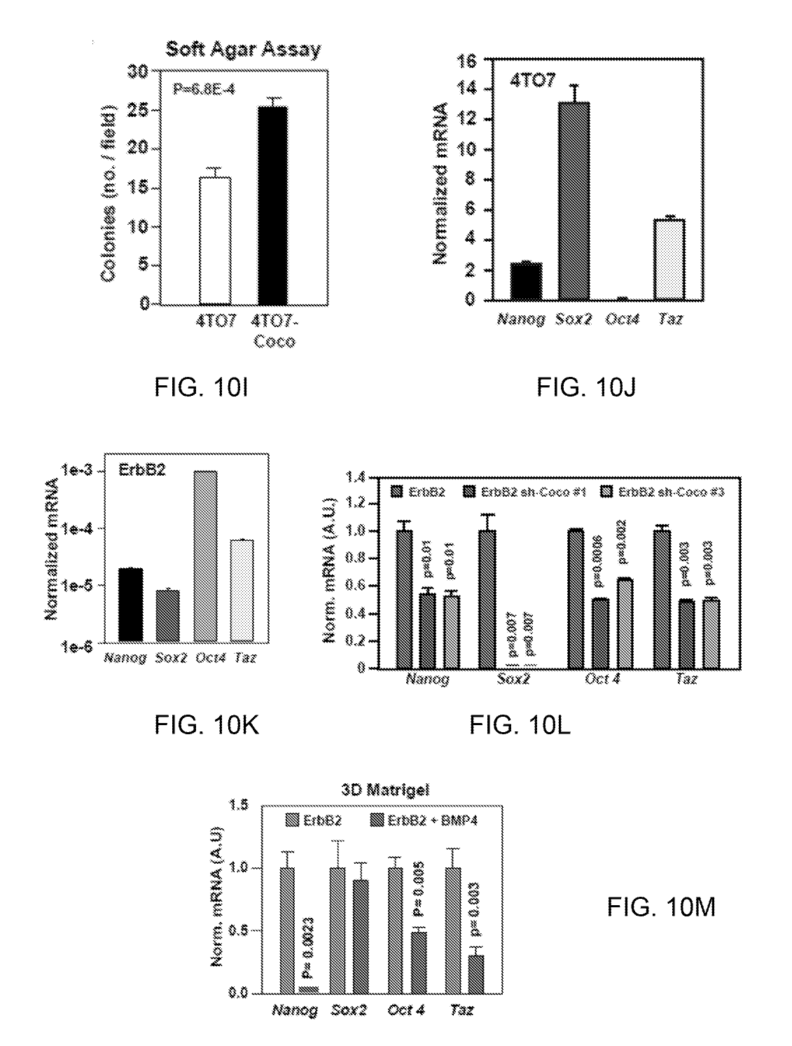

Since the disseminated tumor cells that are fated to outgrow into metastases, the metastasis-initiating cells, share functional properties with cancer stem cells (Nguyen et al., 2009; Valastyan and Weinberg, 2011), we considered the possibility that Coco promotes the manifestation of cancer stem cell traits. FACS analysis revealed that the 4T07 cells and the 4T1 cells express similarly high levels of the mammary epithelial stem cell markers CD24, CD29, and CD49f (FIG. 10A). In addition, the transcriptomic profile of the indicated mammary carcinoma lines was compared to that of normal mammary stem cells, luminal progenitors, and mature luminal cells. DNA microarray analysis indicated that they are endowed with a program of gene expression similar to that of normal mammary epithelial stem cells (see Table 2).

TABLE 2
Molecular characterization of mammary carcinoma lines

| Comparison | Correlation coefficient | p-value |
|---|---|---|
| 4TO7 vs. luminal progenitor | 0.019076068 | 0.914056275 |
| 4TO7 vs. mature luminal cell | 0.047379132 | 0.625434026 |
| 4TO7 vs. mammary stem cell | 0.295191807 | 9.01E−11 |
| 4TO7 vs. luminal progenitor | −0.030013322 | 0.865142264 |
| 4TO7 vs. mature luminal cell | 0.065894856 | 0.496875853 |
| 4TO7 vs. mammary stem cell | 0.279205209 | 9.88E−10 |

Finally, immunoblotting and immunofluorescence analysis indicated that both types of cells do not express markers associated with normal bi-potential progenitors (CK18/CK14, CK19/CK14) or with cells differentiated along the luminal (CK8, ERα, PR, Muc1, β-casein, ZO-1) or myoepithelial lineage (CK5, SMα-actin, integrin β4, laminin γ2, p63, CD10), but they express EpCAM, low levels of ErbB2 and very low levels of the transcription factor GATA3, a master regulator of luminal differentiation (Table 3).

TABLE 3
Phenotypic characterization of mammary carcinoma lines

| | | | 4TO7 + | | | |
|---|---|---|---|---|---|---|
| | | 4TO7 | BMP | 4T1 | MCF-7 | MCF-10A |
| Bipotential | CK18/CK14 | − | − | − | − | − |
| | CK19/CK14 | − | − | − | − | − |
| Luminal | EpCAM | +++ | +++ | +++ | +++ | n.d. |
| Progenitor | ErbB2 | −/+ | −/+ | −/+ | + | n.d. |
| Luminal | GATA3 | −/+ | ++ | −/+ | ++ | n.d. |
| | CK8 | − | − | − | +++ | n.d. |
| | CK18 | − | − | − | +++ | n.d. |
| | CK19 | − | − | − | +++ | n.d. |
| | ERα | − | − | − | +++ | n.d. |
| | PR | − | − | − | + | n.d. |
| | Muc1 | − | − | − | −/+ | n.d. |
| | β-casein | − | − | − | −/+ | n.d. |
| | ZO-1 | − | − | − | + | n.d. |
| Basal | CK5 | − | − | − | n.d. | + |
| | CK14 | − | − | − | n.d. | + |
| | SM-actin | − | − | − | n.d. | + |
| | Integrin β4 | − | − | − | n.d. | + |
| | Laminin γ2 | − | − | − | n.d. | + |
| | P63 | − | − | − | n.d. | + |
| | CD10 | − | − | − | n.d. | + |

− no staining
−/+ borderline detectable
+ weak expression
++ moderate expression
+++ strong expression
n.d. not determined

This marker profile is very similar to that displayed by tumor-initiating cells isolated from mouse models of ErbB2-mediated mammary tumorigenesis (Liu et al., 2007; Lo et al., 2011).

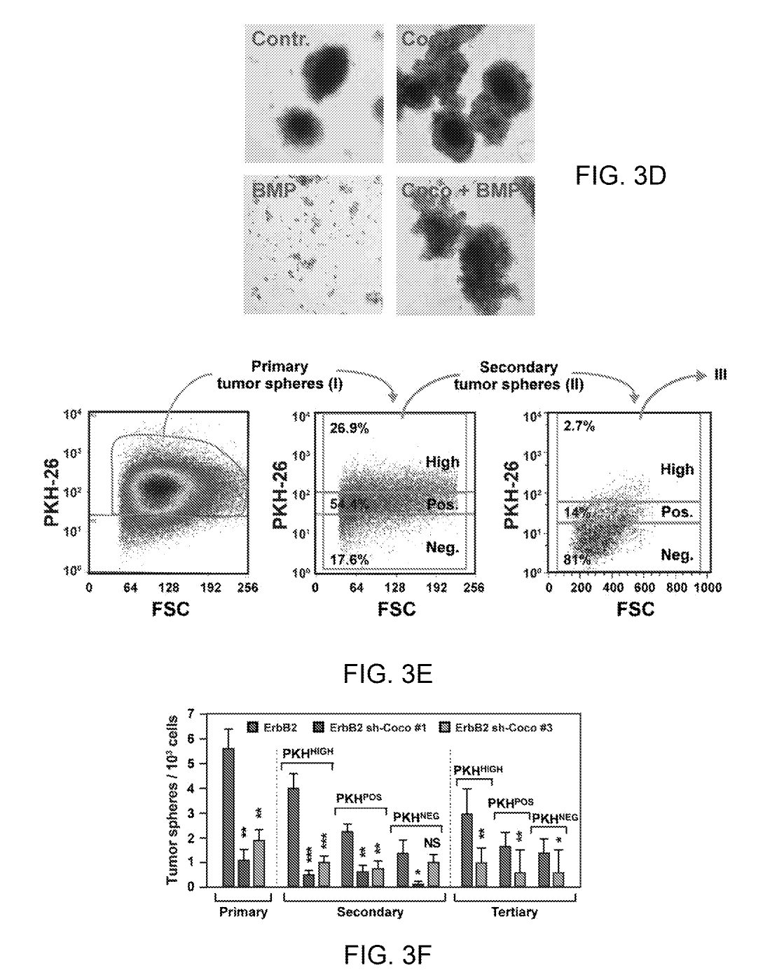

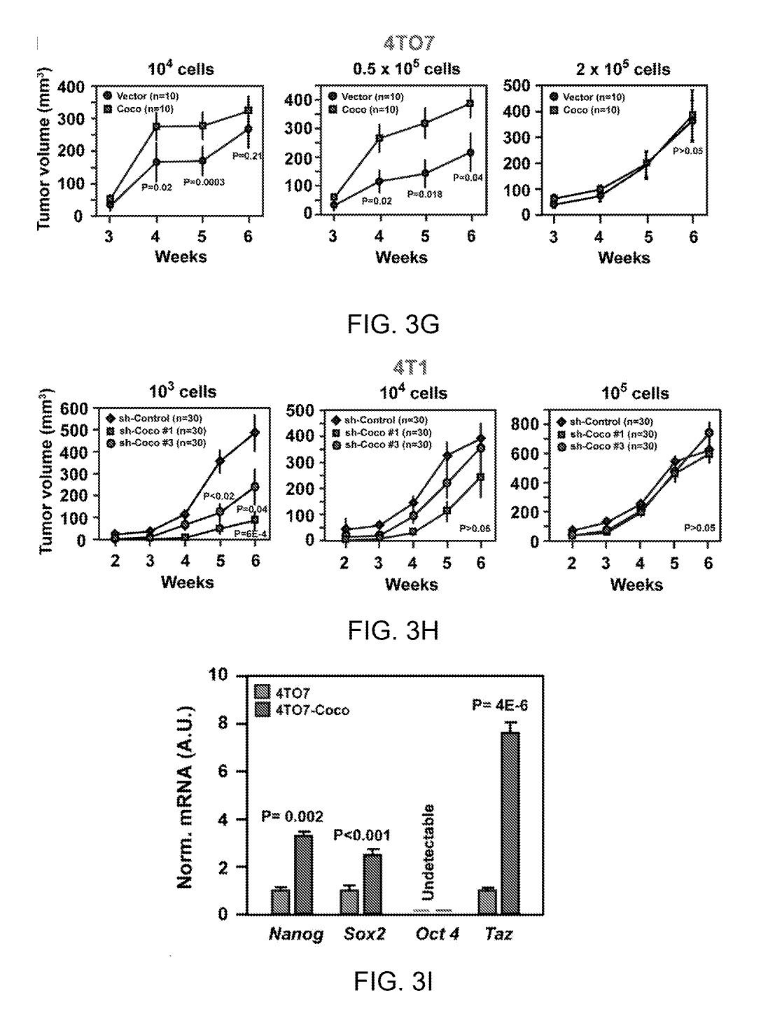

The cancer stem cells are defined by an inherent capability to undergo self-renewal, to give rise to an aberrantly differentiated progeny, and to seed tumors in vivo (Clevers, 2011; Gupta et al., 2009; Shackleton et al., 2009). To examine the hypothesis that Coco promotes exit from dormancy by affecting cancer stem cell function, the ability of Coco to influence the ability of 4T07 cells to form mammospheres under non-adherent conditions was examined. This assay is thought to measure the ability of mammary epithelial stem cells to undergo self-renewal in vitro (Dontu et al., 2003). The 4T07 cells expressing Coco formed approximately twice as many tumor spheres as compared to control cells at each of three subsequent passages (FIGS. 3A and 10B). Similarly, administration of recombinant Coco to naïve 4T07 cells induced a dose-dependent increase in tumor sphere seeding efficiency (FIG. 3B). Cells exposed to the maximal dose of Coco tested, 0.9 □g/ml, formed as many tumor spheres as those transduced with the vector encoding Coco. Since the 4T07 cells express low levels of the mRNAs encoding BMP2 and BMP5 (FIG. 9L), Coco may expand the ability of 4T07 cells to give rise to tumor spheres by blocking the small amounts of BMP that these cells produce. In agreement with this interpretation, treatment of naïve 4T07 cells with 10 ng/ml of exogenous BMP4 caused a profound inhibition of tumor sphere formation, which was reversed by concurrent administration of Coco (FIGS. 3C and 3D).

FACS analysis after propidium iodide staining indicated that Coco does not increase the ability of 4T07 cells to survive or modifies their distribution in the various phases of the cell cycle under the conditions of the mammosphere assay (FIG. 10C). BMP4 caused a very modest increase in apoptosis (1.6% as compared to 0.34% cells with sub-G1 DNA content) but also in the proportion of cells in S-phase (from 11.9% to 13.4%). These small changes were reversed by concurrent administration of Coco (FIG. 10C). In addition, although BMP4 completely disrupted tumor sphere formation, it did not reduce the total number of live cells present at the end of the assay as measured by MTT assay (not shown). These results suggest that Coco-mediated blockage of BMP does not promote tumor sphere formation by increasing tumor cell survival or proliferation, but rather it specifically affects the capability of tumor sphere-forming cells to outgrow under the conditions of the assay.

Next, whether BMP can cause partial or aberrant differentiation of breast cancer cells was evaluated. Interestingly, treatment of 4T07 cells with BMP4 induced expression of the transcription factor GATA3, a master regulator of luminal differentiation, whereas concurrent administration of recombinant Coco reversed this process (FIG. 10F). In addition, whereas the 4T07 cells that became dormant upon extravasation in the lung stroma displayed strong reactivity for GATA3 irrespective of whether they expressed Coco, the metastatic outgrowths formed by the 4T07 cells expressing Coco were uniformly negative for GATA3 (FIG. 10G). These results suggest that the high levels of BMP present in the lung stroma induce the 4T07 cells to express GATA3, and they further imply that expression of Coco reverses this process in tumor cells fated to give rise to metastatic outgrowths. However, additional experiments indicated that BMP4 is not able to induce expression in vitro of several markers associated with differentiated luminal cells (Table 2). Furthermore, DNA microarray analysis indicated that treatment with BMP4 is not sufficient to induce expression of differentiated genes in 4T07 cells placed in 3D Matrigel under conditions that favor the differentiation of normal mammary epithelial cells (not shown). These results suggest that BMP can poise breast cancer cells toward luminal differentiation by inducing GATA3, but is insufficient to induce partial or aberrant differentiation en face of the ability of oncogenic signalling to block differentiation.

To confirm and extend these results, primary tumor cells isolated from MMTV-Neu(YD) mice were examined. Prior studies have suggested that transgenic ErbB2 mammary tumors follow a cancer stem cell model (Cicalese et al., 2009). The tumor spheres formed by their constituent primary cells derive from the clonal expansion of single cells endowed with the ability to undergo self-renewal in vitro. Moreover, staining with the lipophilic dye PKH-26, which is diluted after each cell division, has indicated that the cells that retain the dye during the mammosphere assay possess the highest self-renewal capacity in vitro and the highest tumor initiation capacity in vivo (Cicalese et al., 2009). To examine if Coco affects self-renewal in vitro, Coco-silenced and control ErbB2-transformed cells were stained with PKH-26 and subjected to primary tumor sphere assay. The primary spheres were dissociated and their constituent cells were sorted according to staining intensity (PKH^{HIGH}, PKH^{POS}, and PKH^{NEG}) and subjected to secondary tumor sphere assay. Finally, the secondary tumor spheres were subjected to the same protocol to derive tertiary tumor spheres (FIG. 3E). As previously reported, replating of the PKH^{HIGH}, PKH^{LOW}, and PKH^{NEG} subsets led to tumor sphere formation in all cases, albeit with decreasing efficiency. Notably, knock down of Coco inhibited tumor sphere formation at each passage (FIG. 3F). Treatment of naïve ErbB2-transformed cells with BMP4 exerted the same effect and Coco reversed it (FIG. 10D). FACS analysis of propidium iodide-stained cells indicated that silencing of Coco or treatment with BMP does not inhibit tumor sphere formation by decreasing tumor cell survival or proliferation (FIG. 10E). In addition, silencing of Coco did not alter the level of expression of the stem cell markers Sca-1, CD24, and CD49f in these ErbB2-transformed cells (not shown). Finally, DNA microarray analysis indicated that BMP4 does not induce expression of differentiated genes when these cells are plated in 3D Matrigel (not shown). These results suggest that Coco selectively sustains the ability of ErbB2-transformed cells to undergo self-renewal in vitro.

### Example 6

### Coco Promotes Tumor Initiation In Vivo

Having found that Coco enhances the ability of breast cancer cell to form tumor spheres in vitro, we examined if it also enhanced clonogenic outgrowth in vitro and tumor initiation in vivo. We found that Coco increases clonogenic outgrowth under standard culture conditions as well as in soft agar (FIGS. 10H and 10I). Furthermore, Coco increased the ability of 4T07 cells to seed tumors in vivo (FIG. 3G). In agreement with the hypothesis that Coco affects tumor initiation capacity but not subsequent tumor growth, this effect was evident only when limiting numbers of tumor cells were injected in the mammary fat pad (10⁴ and 5×10⁴ cells, but not 2×10⁵ cells). Conversely, silencing of Coco inhibited the capacity of 4T1 cells to initiate orthotopic tumors under similar conditions (10³ cells, but not 10⁴ or 10⁵ cells (FIG. 3H). Semiquantitative RT-PCR experiments indicated that while the normal cell types present in the mammary fat pad express BMP7, the primary tumors generated by 4T07 or 4T1 cells injected at this site did not express any of the genes encoding BMP proteins (FIG. 9L). These findings suggest that Coco promotes tumor initiation in the mammary gland as well as reactivation at lung metastatic sites because it opposes the ability of BMP to block clonogenic outgrowth at both sites.

### Example 7

### Coco Sustains Expression of Stem Cell Transcription Factors

Since Coco-mediated inhibition of endogenous BMP did not cause changes in the levels of stem cell markers at the surface of 4T07 or ErbB2-transformed cells, we asked whether it modulated the expression of transcription factors implicated in cancer stem cell activity. Recent studies have indicated that the transcriptional program regulated by the embryonic stem cell transcription factors Sox2, Nanog, and Oct4 is often reactivated in aggressive and metastatic human carcinomas, including breast cancers (Ben-Porath et al., 2008; Wong et al., 2008). Furthermore, the transcriptional coactivator Taz, which is inhibited by the Hippo tumor suppressor pathway, has been implicated in breast cancer stem cell maintenance (Cordenonsi et al., 2011). Q-PCR experiments indicated that the 4T07 cells express high levels of Sox2 and lower levels of Taz and Nanog, whereas the ErbB2-transformed cells express high levels of Oct4 and lower levels of the remaining three transcriptional regulators (FIGS. 10J and 10K). Notably, expression of Coco increased the levels of expression of Nanog, Sox2, and Taz in 4T07 cells (FIG. 3I). In contrast, treatment with BMP completely suppressed their expression (FIG. 3J). Furthermore, silencing of Coco significantly reduced the level of expression of Nanog, Oct4, and Taz and completely ablated expression of Sox2 in ErbB2-transformed cells (FIG. 10L). Since Nanog, Sox2, and Oct4 are part of a self-sustaining circuit that powers stem cell maintenance (Young, 2011) and Taz appears to be specifically required in breast tumor progenitor cells (Cordenonsi et al., 2011), Coco may contribute to the manifestation of breast cancer stem cell traits by influencing the expression of these transcription factors.

### Example 8

### Coco Promotes Tumor Sphere Formation In Vitro and Metastatic Reactivation In Vivo by Inhibiting BMP Signaling

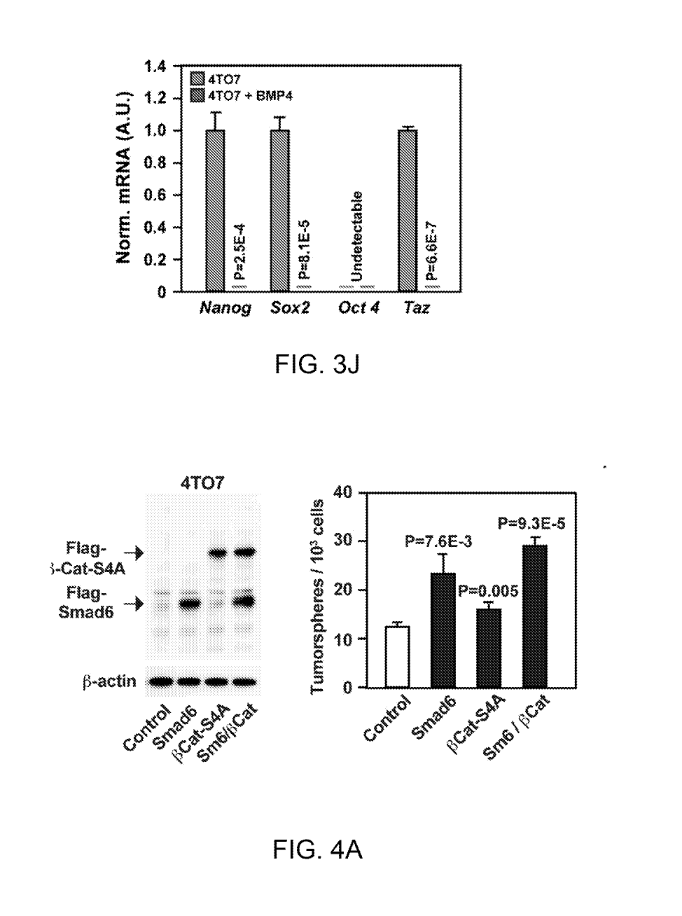

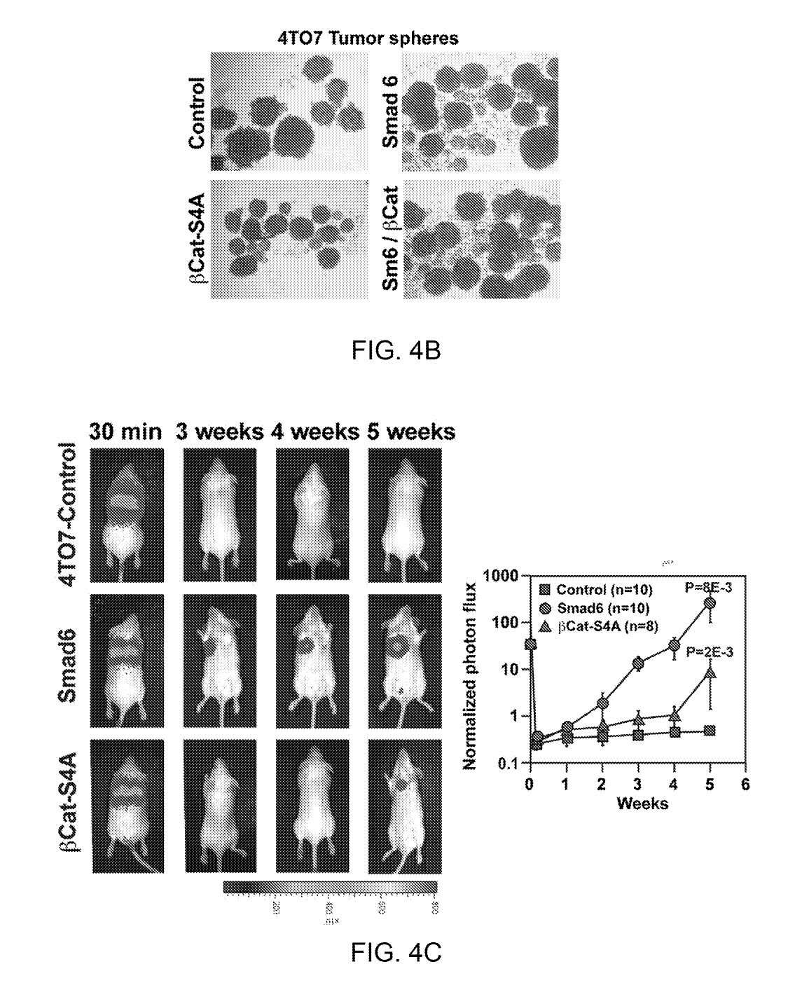

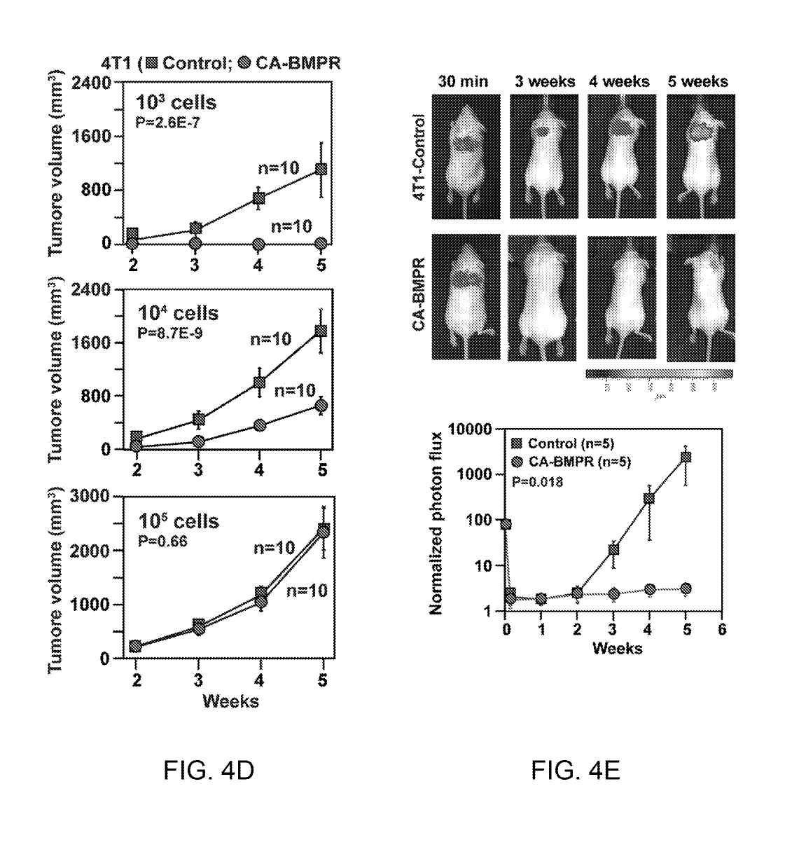

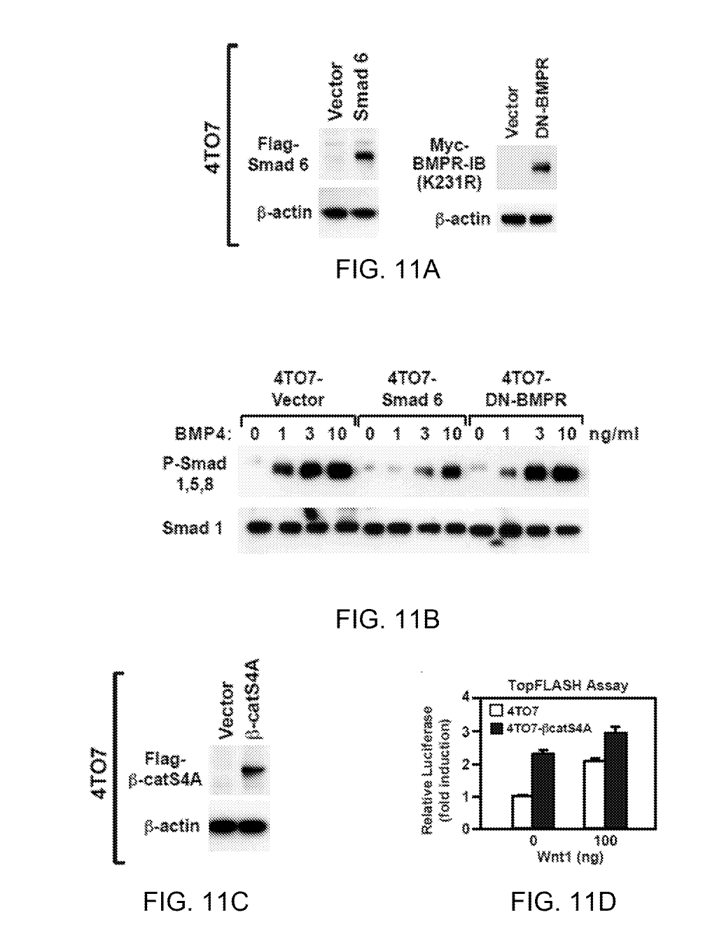

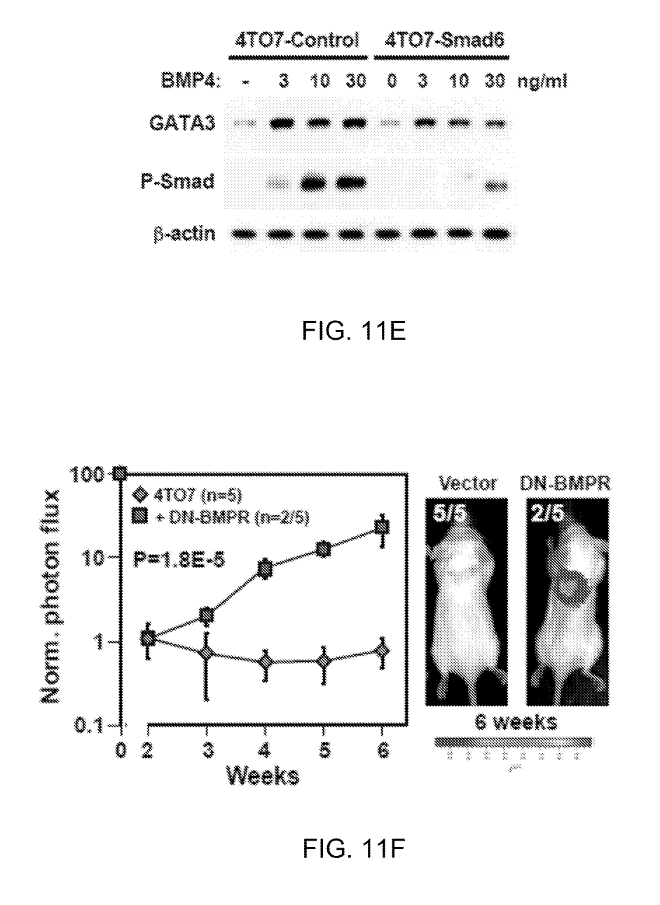

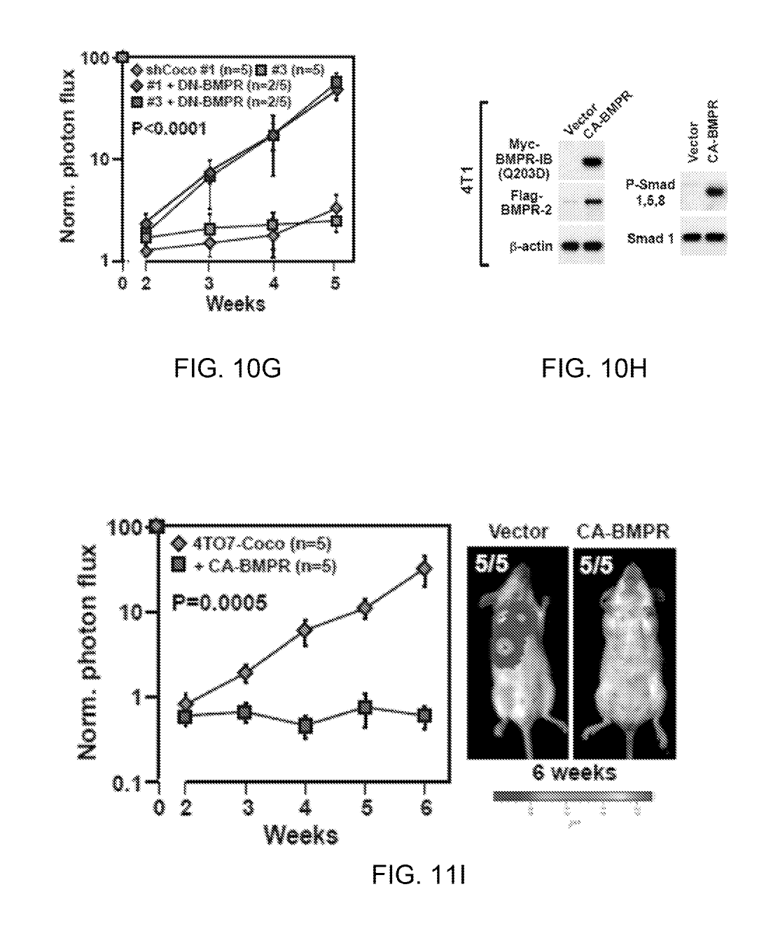

To examine the mechanism through which Coco enhances the capability of metastasis-initiating cells to outgrow in the tumor sphere assay in vitro and during lung colonization in vivo, 4T07 cells were transduced with Smad6, which inhibits canonical BMP signalling (Hata et al.), with the activated β-catenin mutant S4A, or with both Smad6 and β-catenin-S4A (FIG. 4A, left). Control experiments confirmed that expression of Smad6 inhibits C-terminal phosphorylation of Smad 1, 5, 8 in response to BMP, whereas expression of β-catenin-S4A activates the Wnt-responsive TopFLASH reporter in 4T07 cells (FIGS. 11A-D). Smad6 increased the capability of 4T07 cells to form tumor spheres by approximately twofold, similar to the effect of Coco expression. In contrast, activated β-catenin did not induce this effect, either alone or in cooperation with Smad6 (FIGS. 4A and 4B). Furthermore, Smad6 partially attenuated the induction of GATA3 in response to exogenous BMP (FIG. 11E). The ability of Smad6 to replicate the effects that Coco exerts on stem cell behavior suggests that Coco functions predominantly, if not exclusively, by inhibiting BMP signaling.

To examine if inhibition of BMP signaling promotes metastatic reactivation, 4T07 cells expressing Smad6 or activated β-catenin were injected in the tail vein of mice. Whereas activated β-catenin promoted metastatic colonization to the lung to a modest extent, Smad6 induced this process as efficiently as Coco (FIG. 4C, see also FIG. 1G). In general agreement with the observation that expression of a dominant negative form of BMPR accelerates progression to lung metastasis in MMTV-PyMT mice (Owens et al., 2011), expression of a dominant negative BMPR-IB induced the 4T07 cells to colonize the lung after tail vein injection. However, this construct inhibited BMP signaling and therefore promoted lung metastasis less efficiently as compared to Smad6 (FIGS. 11A, 11B, and 11F). Dominant negative BMPR-IB exerted a similar effect in Coco-silenced 4T1 cells (FIG. 11G). These results suggest that inhibition of BMP signaling is sufficient to rescue 4T07 cells and Coco-silenced 4T1 cells from tumor dormancy.

As an alternative approach, we examined if expression of a constitutively active BMP receptor decreased the ability of 4T1 cells to initiate tumorigenesis. To test this hypothesis, an activated form of BMPR-IB (Q203D) was co-expressed together with BMPR-II (jointly termed CA-BMPR) in these cells. The 4T1 cells expressing CA-BMPR exhibited constitutive C-terminal phosphorylation of Smad 1, 5, 8 (FIG. 11H) and were significantly less tumorigenic upon injection in the mammary fat pad of syngeneic mice as compared to vector-transduced controls (FIG. 4D). Whereas 10³ control cells gave rise to sizeable tumors (>1 cc at 5 weeks) and 10⁴ control cells produced large tumors (>1.75 cc at 5 weeks), 10³ cells expressing CA-BMPR were non tumorigenic and 10⁴ cells expressing CA-BMPR gave rise to relatively small tumors (<0.75 cc at 5 weeks). When 10⁵ cells were injected, both control 4T1 cells and those expressing CA-BMPR gave rise to similarly large tumors (FIG. 4D). These results indicate that BMP signaling is sufficient to significantly suppress the tumor initiating capacity of 4T1 cells.

To further study the connection between tumor initiating capacity and metastatic outgrowth, we examined the ability of 4T1 cells expressing CA-BMPR to metastasize to the lung upon tail vein injection. Whereas control 4T1 cells were highly metastatic in this assay, those expressing CA-BMPR were unable to colonize the lung (FIG. 4E), confirming that BMP signaling opposes metastatic colonization. The constitutively active BMPR exerted a similar effect in 4T07 cells expressing Coco (FIG. 11I). These findings suggest that Coco induces metastasis-initiating cells to exit from dormancy by alleviating the capacity of lung-derived BMP proteins to activate canonical signalling through Smad 1, 5, 8 proteins.

### Example 9

### Coco Promotes Human Breast Cancer Metastasis

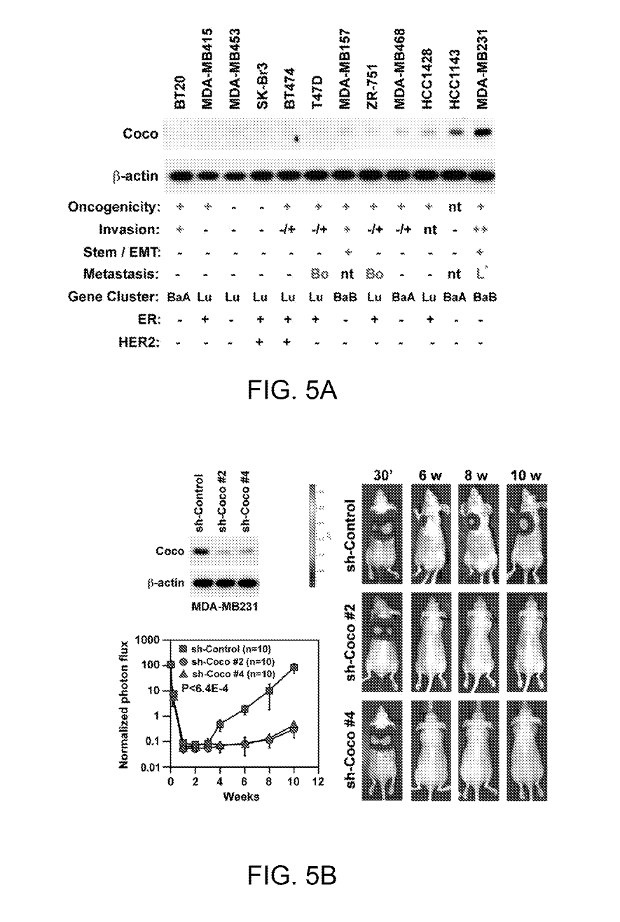

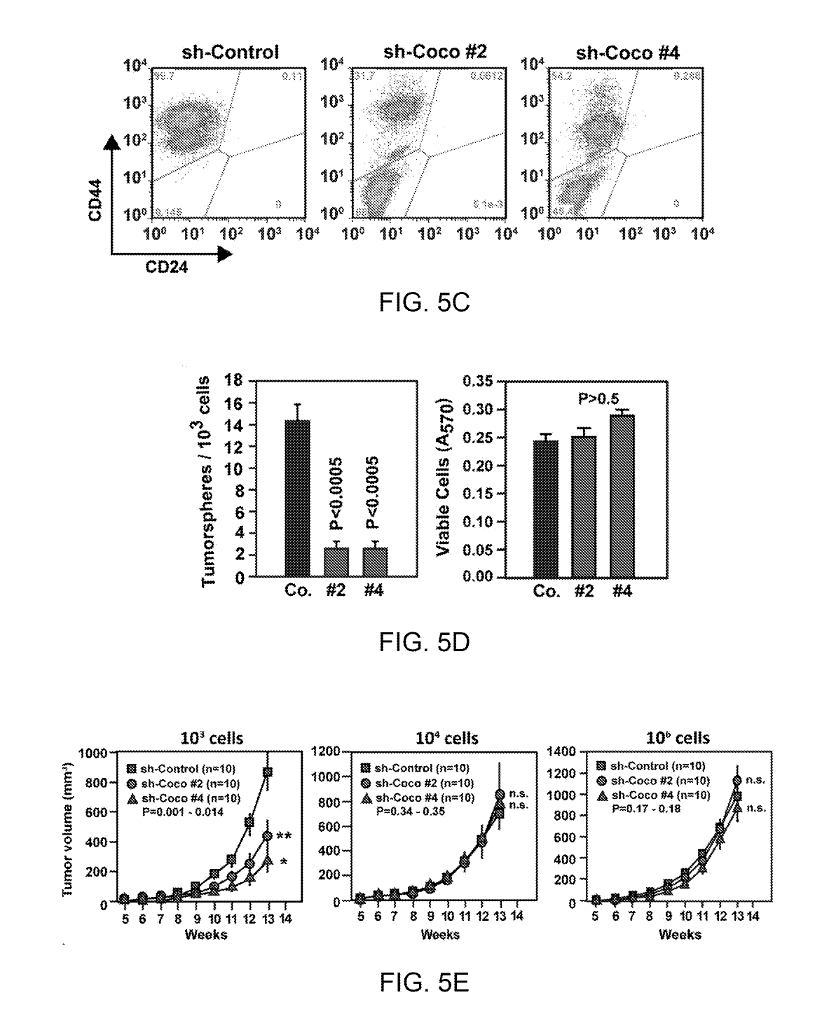

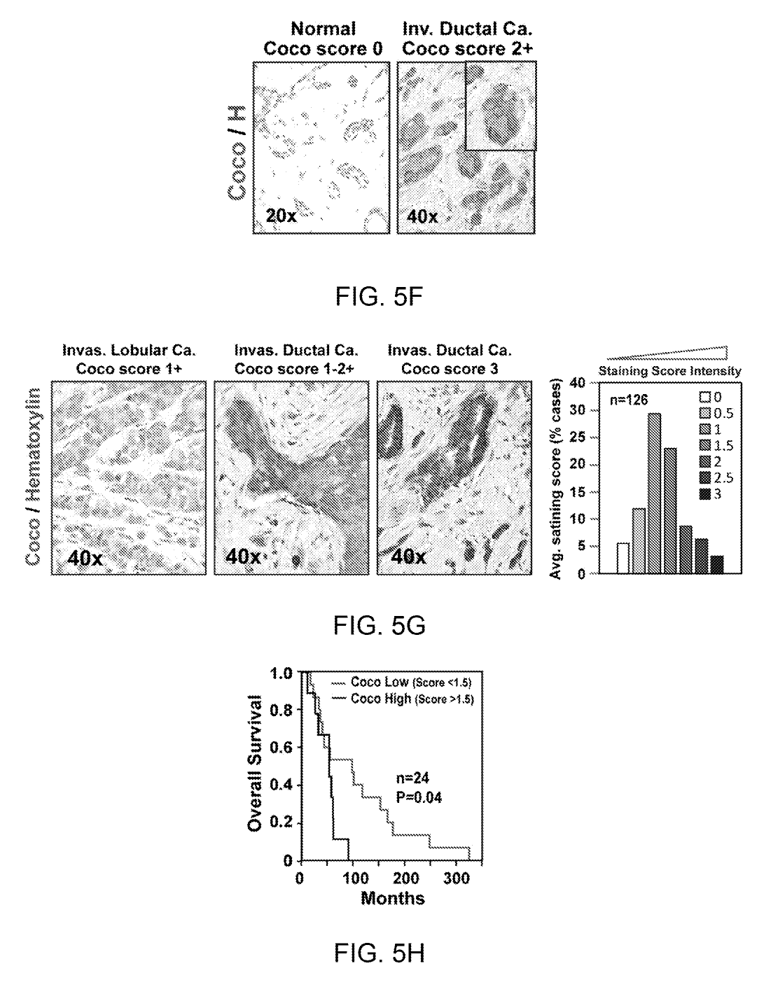

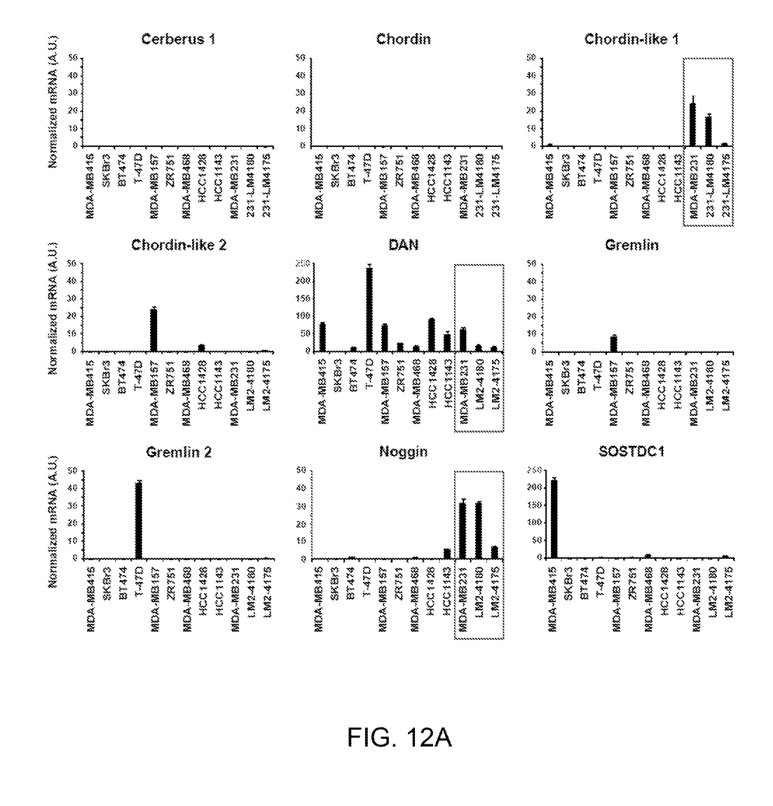

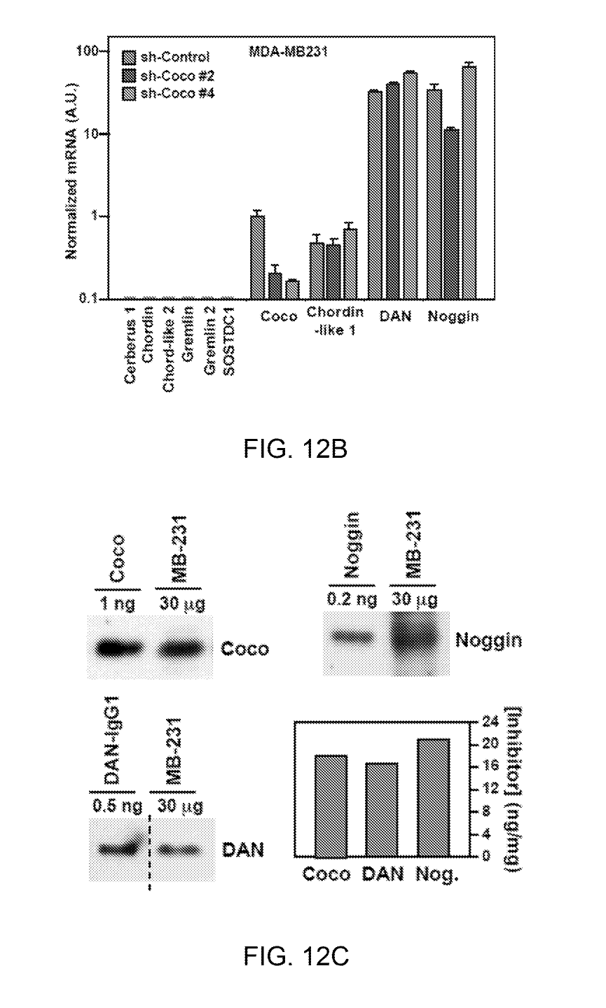

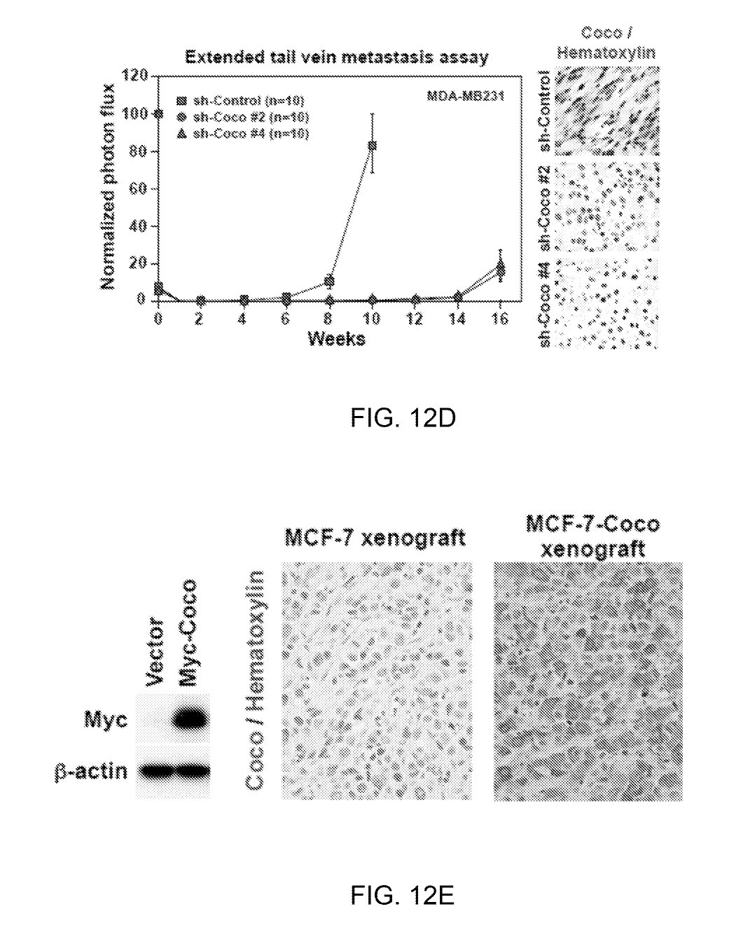

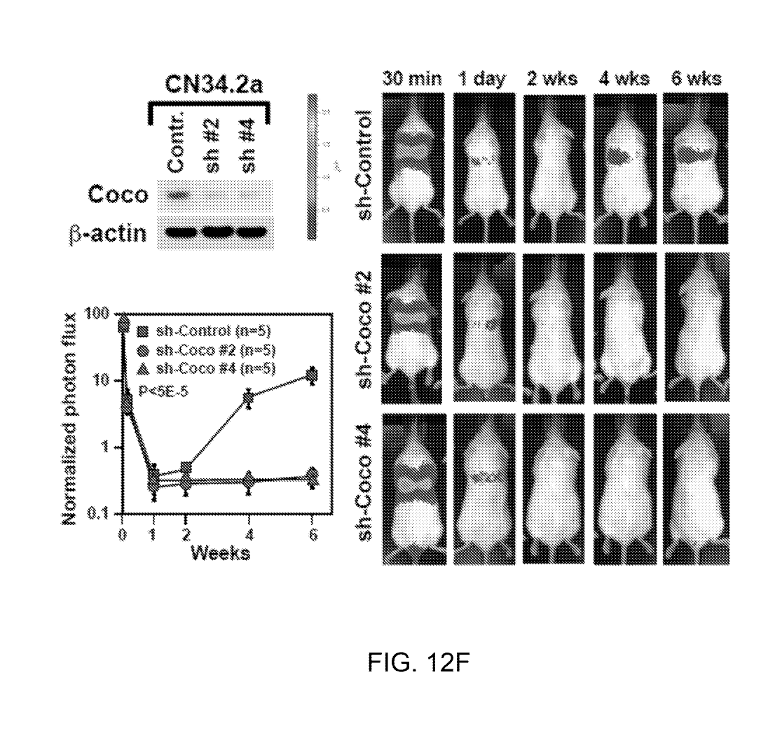

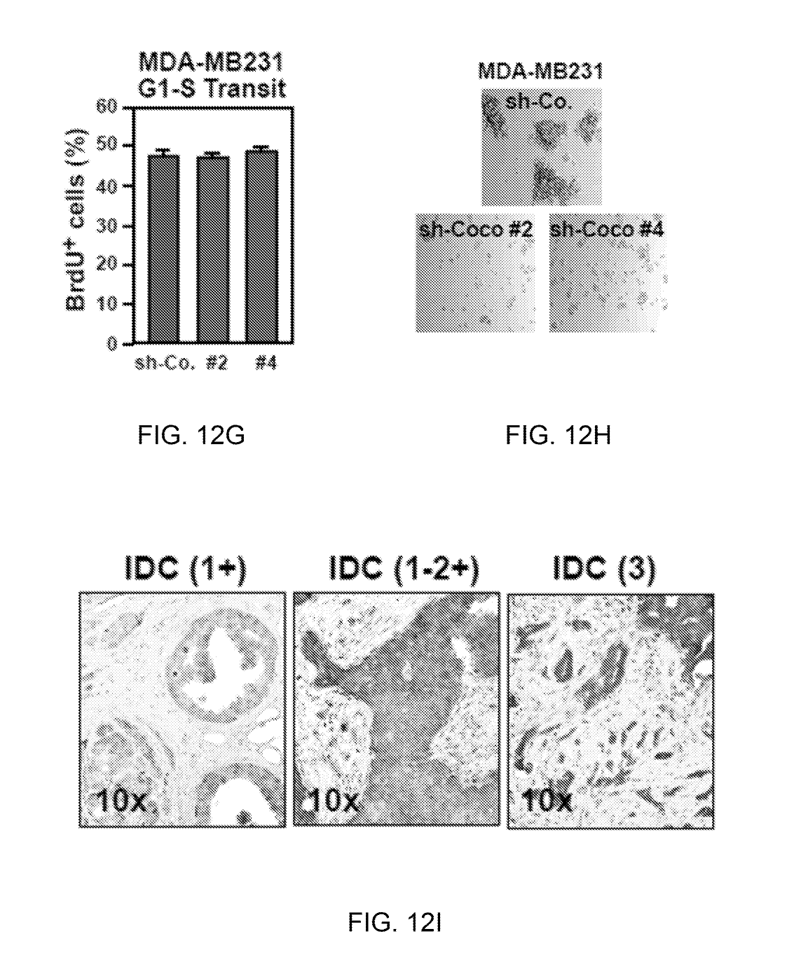

To explore the role of Coco in human breast cancer metastasis, we examined the expression of the protein in a panel of 12 human breast cancer cell lines. Interestingly, Coco was expressed at low or undetectable levels in non-tumorigenic (MDA-MB453 and SK-Br3) (Lacroix and Leclercq, 2004) or non-invasive cell lines (HCC1143) (Neve et al., 2006) as well as in ER+ cell lines capable of colonizing the bone upon intracardiac injection in estrogen-supplemented mice (T47D and ZR-751) (Yin et al., 2003) (FIG. 5A). In contrast, high levels of Coco were detected in the highly metastatic MDA-MB231 cells, which can colonize the lung efficiently upon tail-vein injection, and less efficiently, the bone and brain upon intracardiac injection (Bos et al., 2009) and belong to the basal B gene expression cluster associated with a stem cell phenotype (Blick et al., 2010) (FIG. 5A). Q-PCR analysis indicated that 3 of the 9 additional secreted BMP inhibitors, Noggin, DAN and Chordin-like 1, were also selectively expressed in MDA-MB231 cells (FIG. 12A).

To test if Coco mediates human breast cancer metastasis to the lung, Coco was silenced in MDA-MB231 cells using two distinct shRNAs. As anticipated, silencing of Coco downregulated the expression of Coco at the mRNA and protein level without affecting the level of expression of Noggin, DAN, and Chordin-like 1 (FIGS. 5B and 12B). Although MDA-MB231 cells express levels of Noggin and DAN proteins and Chordin-like 1 mRNA comparable to those of Coco (FIGS. 12B and 12C), silencing of Coco with 2 distinct shRNAs was sufficient to suppress the ability of these cells to colonize the lung upon tail-vein injection (FIG. 5B). Monitoring of the mice for 6 additional weeks revealed that the Coco-silenced cells eventually gave rise to micrometastatic lesions (FIG. 12D, left). Since affinity-purified antibodies reacting selectively with Coco (FIG. 12E) did not react with these lesions (FIG. 12D, right), we presume that they did not arise as a result of re-expression of Coco but rather through the acquisition of genetic or epigenetic modifications able to bypass its requirement. To further test the generality of the effect of Coco, we silenced its expression in the CN34.2a cells, which are derived from the pleural effusion of a patient affected by metastatic breast cancer like the MDA-MB231 cells but are less aggressive (Gupta et al., 2007a; Padua et al., 2008). As shown in FIG. 12F, silencing of Coco suppressed lung colonization by these cells. Together with previous observations, these results suggest that Coco is a general mediator of metastatic reactivation in the lung.

Sorting according to the surface expression of CD44 and CD24 has been successfully used to identify cancer stem cells within primary breast tumors or mammary cell lines, including the MDA-MB231 cells (Al-Hajj et al., 2003; Cordenonsi et al., 2011). Whereas the CD44^{HIGH}/CD24^{LOW/-}cells comprise cells endowed with self-renewal and tumor initiating capacity, the remaining cells are thought to represent a more differentiated progeny devoid of stemness. We therefore examined if silencing of Coco influenced the levels of expression of these surface markers in MDA-MB231 cells. As anticipated, the large majority of control cells were CD44^{HIGH}/CD24^{LOW/-}. Silencing of Coco led to the appearance of a large subpopulation of cells that did not express CD44, suggesting that expression of Coco is necessary to maintain a stem cell phenotype in MDA-MB231 (FIG. 5C). In consonance with this observation, silencing of Coco profoundly inhibited the ability of MDA-MB231 cells to form tumor spheres in vitro without affecting overall survival and proliferation (FIGS. 5D, 12G and 12H). Finally, knock down of Coco inhibited the capacity of MDA-MB231 cells to initiate tumorigenesis upon orthotopic injection in NOD-SCID-IL2γR^{-/-} mice (FIG. 5E). These results provide evidence that Coco promotes maintenance of various stem cell traits by MDA-MB231 cells.

To study the expression of Coco in human breast cancer, 3 distinct tissue microarrays comprising 15 normal or dysplastic glands and 126 breast cancers were stained with affinity-purified antibodies reacting selectively with Coco (Table 3 and Experimental Procedures). Whereas normal or displastic mammary epithelial cells did not express detectable levels of Coco, the tumor cells and scattered stromal cells in a large fraction of breast tumors produced variable amounts of Coco (FIGS. 5F, 5G and 12I). The intensity of staining varied from weak to strong (FIGS. 5G and 12I). Statistical analysis did not reveal any correlation between the intensity of staining for Coco and pathological grade, clinical stage, ER, or HER2 status in these samples (not shown). Whereas the primary tumors in the two commercial tissue microarrays are not annotated for metastatic relapse, precluding an analysis of the correlation between intensity of staining and metastasis, the MSKCC tissue microarray comprises primary tumors and lung metastatic lesions from clinically and molecularly annotated cases. Interestingly, Kaplan Meyer analysis of the MSKCC dataset indicated that the patients with primary tumors exhibiting high levels of Coco proteins had a significantly shorter overall survival (46.2+7.7 months) as compared to the remaining patients (104.9+23.2 months)(FIG. 5H). However, we were unable to uncover other correlations, possibly because of the limited number of cases present in this tissue microarray and the paucity of paired samples. These observations are consistent with the hypothesis that tumor cell-intrinsic production of Coco confers a selective advantage during both tumor initiation and progression.

### Example 10

### A Coco-Dependent Gene Expression Signature Predicts Metastatic Relapse to the Lung in Human Breast Cancer

To examine if the level of expression of Coco correlates with metastatic relapse, four existing DNA microarray datasets (MSK82, EMC192, EMC286, and NKI295), which include patients that were followed for several years and are well annotated for metastasis (Bos et al., 2009; Minn et al., 2005; van de Vijver et al., 2002; Wang et al., 2005) were analyzed (Tables 5a and 5b).

TABLE 5a
Clinical characteristics of the cohorts of patient in the MSK82,
EMC 192, EMC286 and NKI295 datasets - site of relapse

| | All | Site of Relapse | | | | | | | | | |
|---|---|---|---|---|---|---|---|---|---|---|---|
| | Tumors | Brain | | Lung | | Bone | | Liver | | Lymph Node | |
| Cohort | Tumors | (%) | | (%) | | (%) | | (%) | | (%) | |
| MSK82 | 82 | 5 | (6.1) | 14 | (17.1) | 14 | (17.1) | NA | | 54 | (65.9) |
| EMC286 | 286 | 10 | (3.5) | 25 | (8.7) | 69 | (24.1) | 15 | (5.2) | 0 | |
| NKI295 | 295 | 13 | (4.4) | 52 | (17.6) | 102 | (34.6) | 49 | (16.6) | 0 | |
| EMC192 | 192 | 22 | (11.5) | 21 | (10.9) | 53 | (27.6) | 41 | (21.4) | 144 | (75) |
| MSK82 + | 560 | 37 | (6.6) | 60 | (10.7) | 136 | (24.3) | 56 | (10) | 198 | (35.4) |
| EMC286 + | | | | | | | | | | | |
| EMC192 | | | | | | | | | | | |
| All Datasets | 855 | 50 | (5.8) | 112 | (13.1) | 238 | (27.8) | 105 | (13.6) | 198 | (23.2) |
| combined | | | | | | | | | | | |

TABLE 4b
Clinical characteristics of the cohorts of patient in the MSK82, EMC192,
EMC286 and NKI295 datasets - treatment

| | | Treatment | | | |
|---|---|---|---|---|---|
| | | Adjuvant | Hormonal | | |
| | All Tumors | Chemotherapy | Therapy | ER− Tumors | ER+ Tumors |
| Cohort | Tumors | (%) | (%) | (%) | (%) |
| MSK82 | 82 | 69 (84.1) | 53 (64.6) | 36 (43.9) | 46 (56.1) |
| EMC286 | 286 | 0 | 0 | 77 (26.9) | 209 (73.1) |
| NKI295 | 295 | 110 (37.3) | 40 (13.6) | 67 (22.7) | 228 (77.3) |
| EMC192 | 192 | 37 (19.3) | 38 (19.8) | 80 (41.7) | 124 (64.6) |
| MSK82 + | 560 | 106 (18.9) | 91 (16.3) | 193 (34.5) | 379 (67.7) |
| EMC286 + | | | | | |
| EMC192 | | | | | |
| All Datasets | 855 | 216 (25.3) | 131 (15.3) | 260 (30.4) | 607 (71) |
| combined | | | | | |

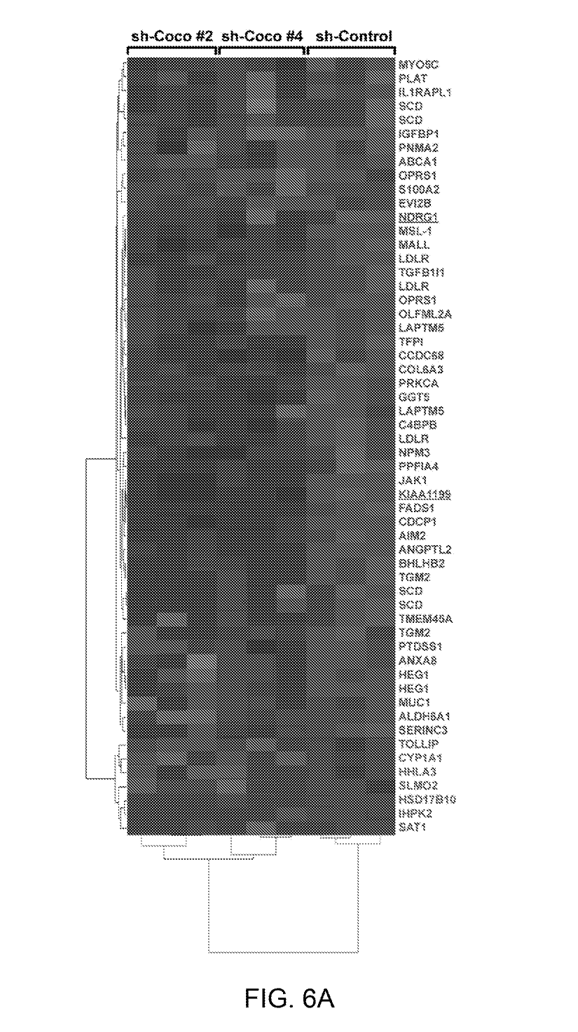

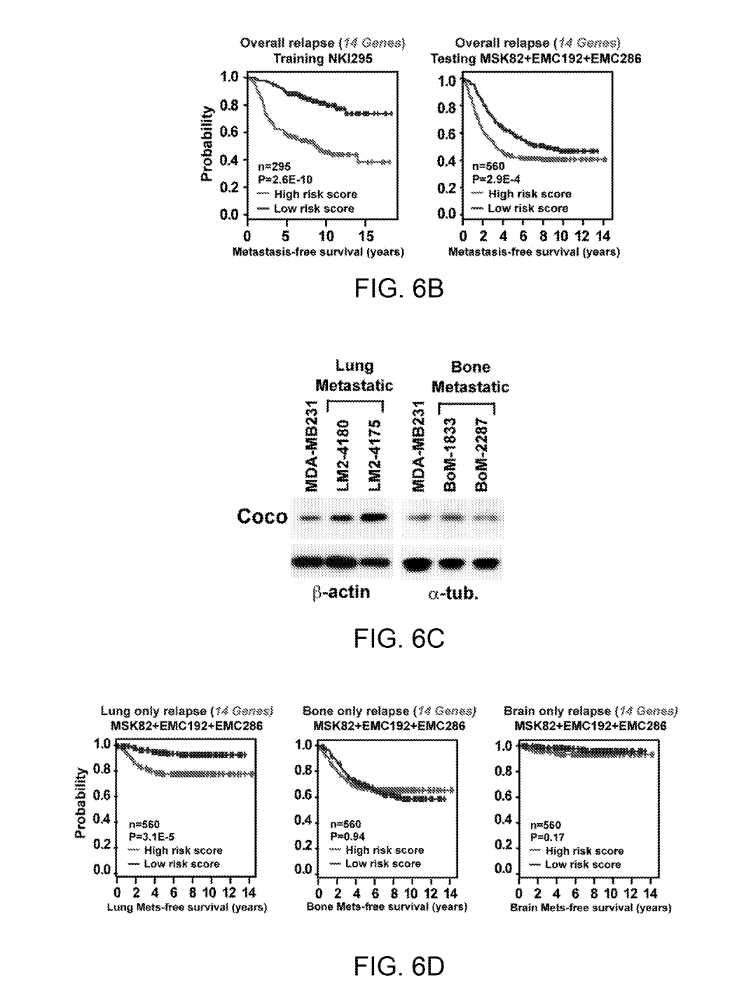

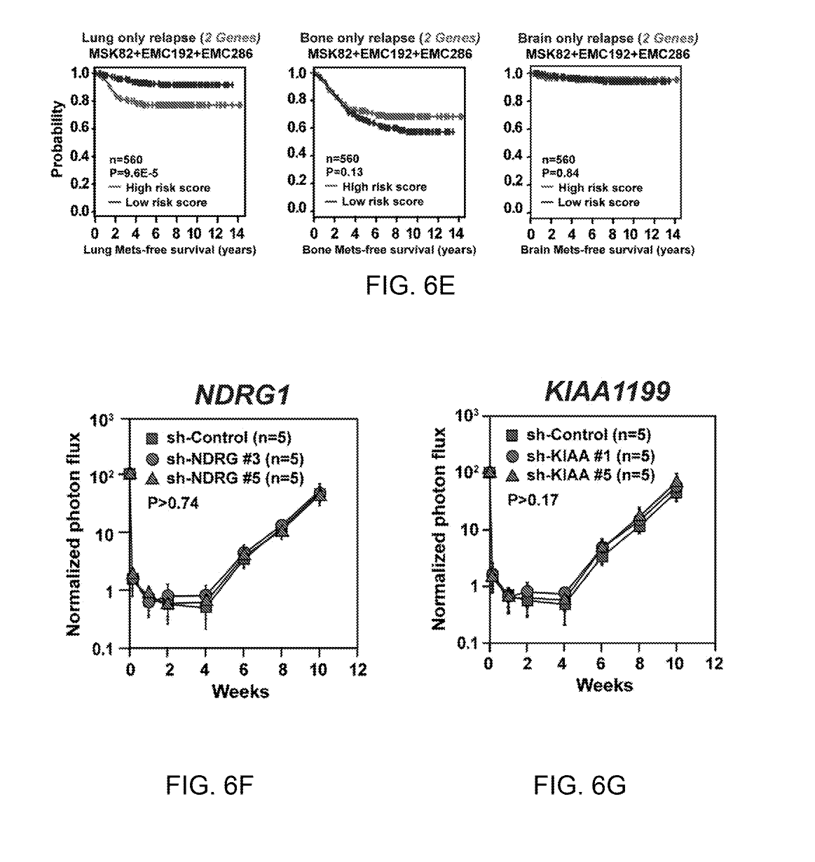

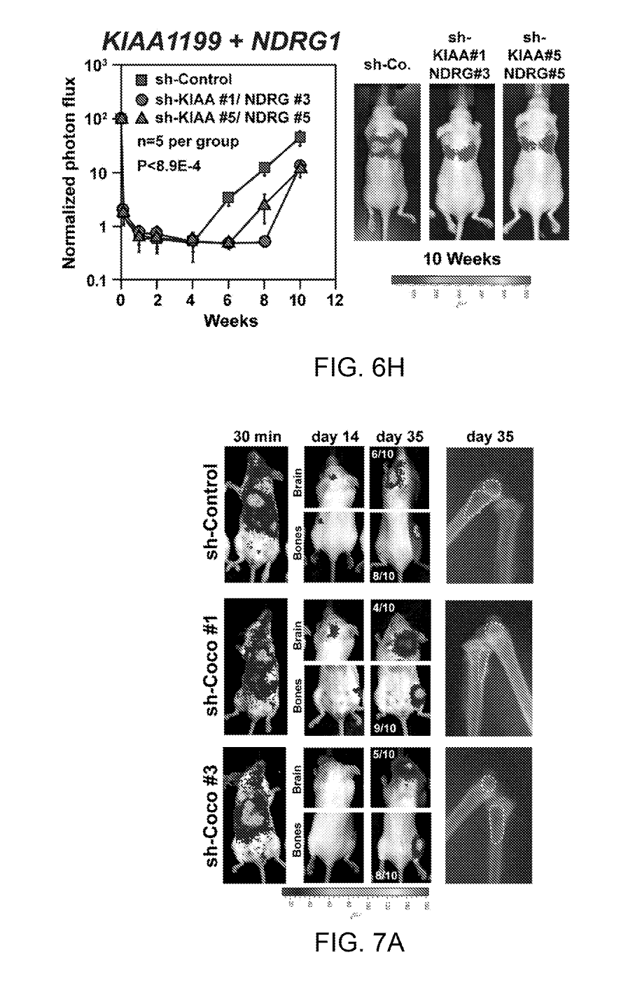

The Affymetrix HG-U133A and Agilent platforms, which were used to build these datasets, do not contain probes for Coco, preventing a direct analysis of the correlation of the expression of Coco with metastatic relapse. We reasoned that the definition of a Coco-dependent signature of gene expression would have provided a more powerful tool to validate the relevance of Coco in human breast cancer. The changes in gene expression caused by silencing of Coco were examined in MDA-MB231 cells in vitro and the resulting signature comprising 56 genes was used as a functional read out of Coco expression (FIG. 6A).

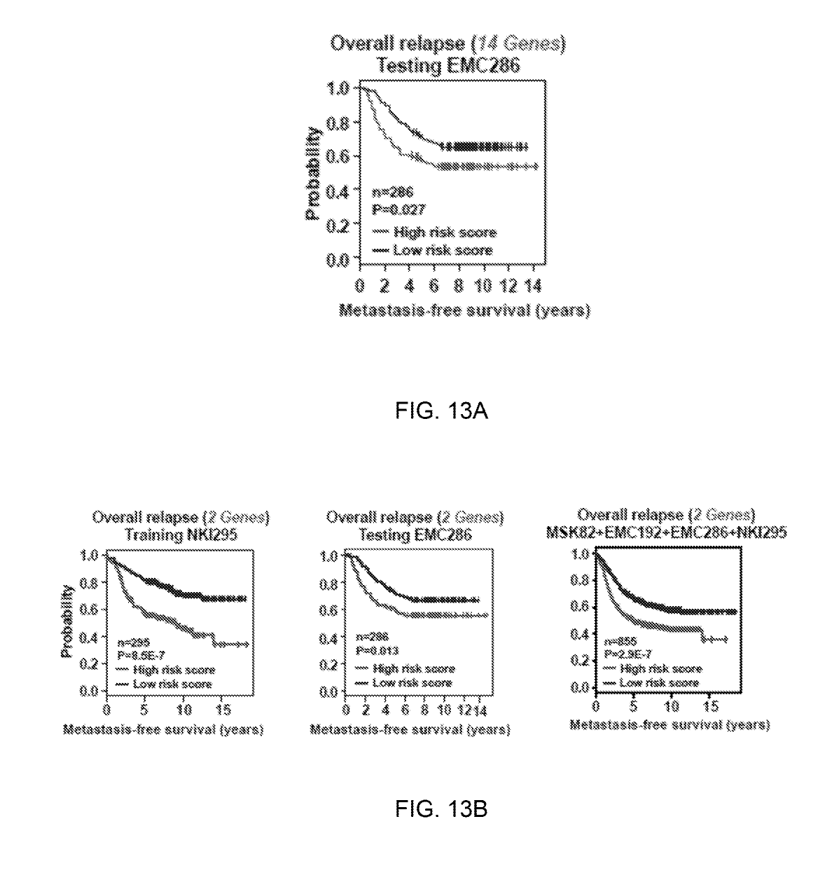

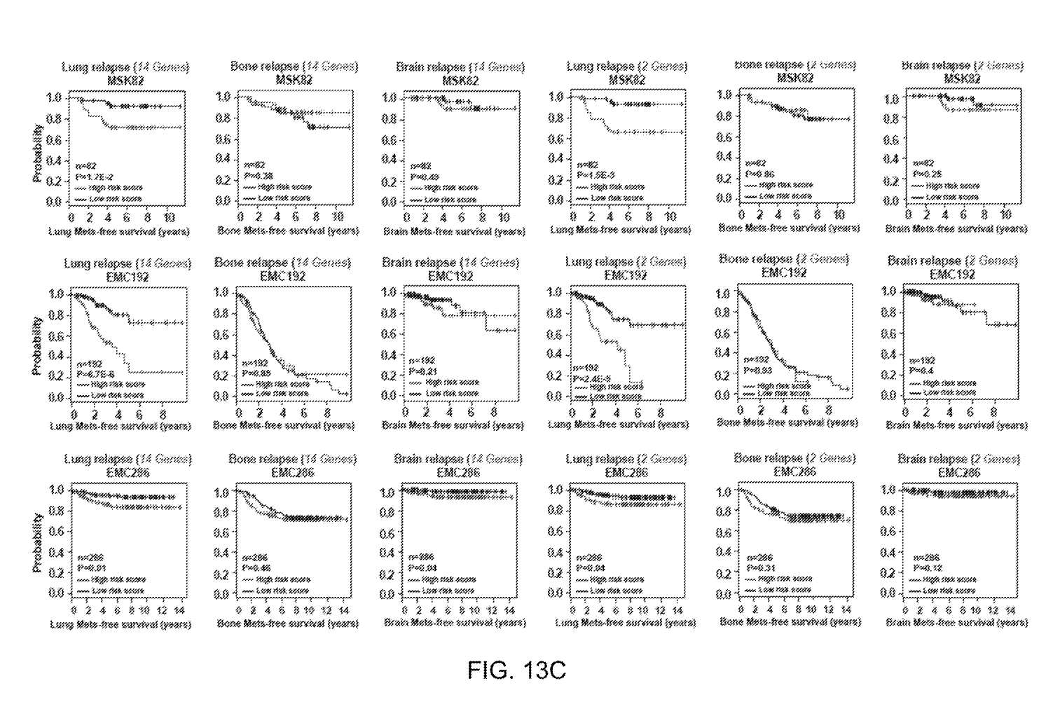

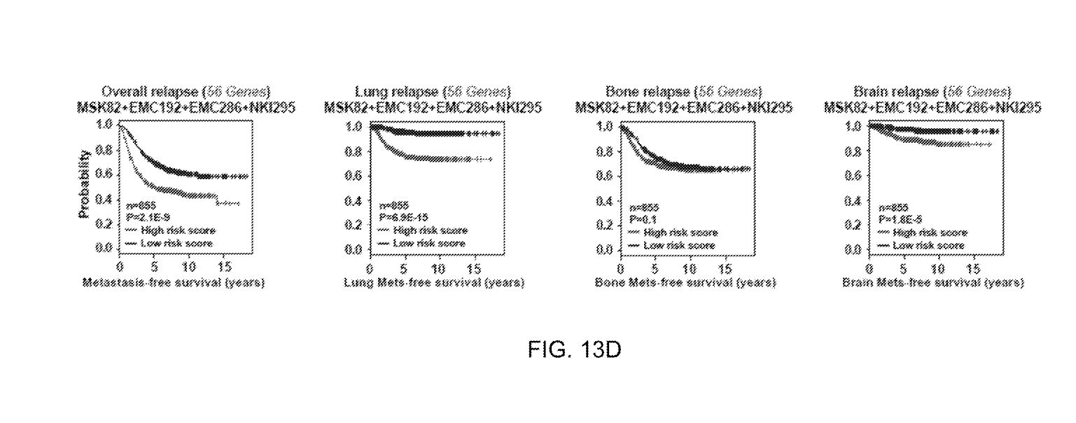

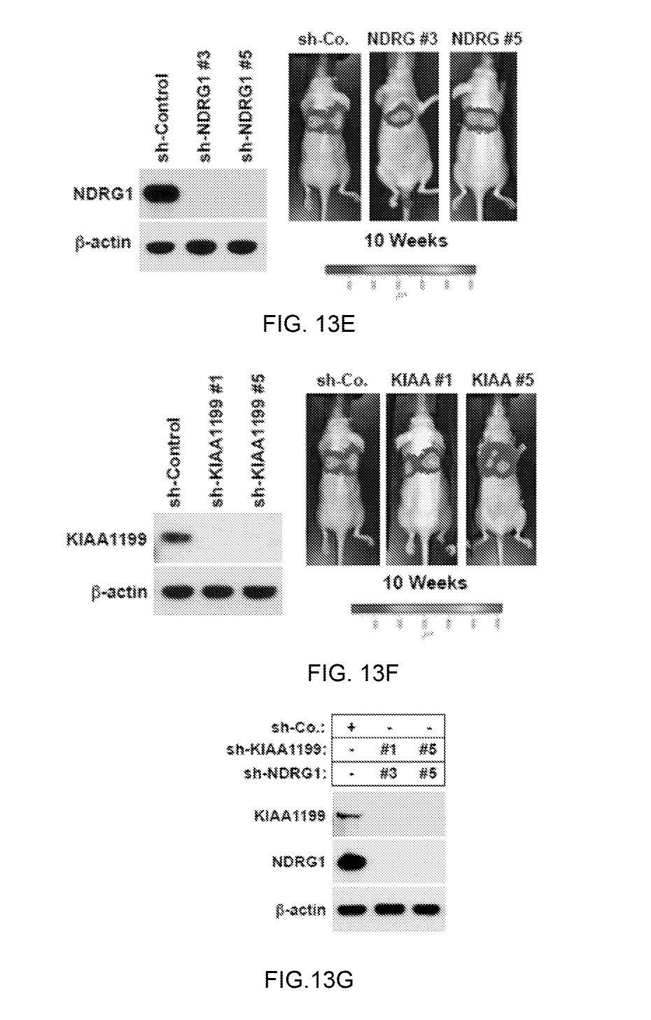

To evaluate the predictive power of the Coco signature, we selected the NKI295 dataset for training because this dataset comprises early stage, lymph-node negative cases (van de Vijver et al., 2002) (Tables 4a and 4b). Using a leave-one-out cross validation method, we obtained an optimal risk score from the NKI295 dataset based on the expression profile of the 14 genes most relevant in predicting overall metastatic relapse (FIG. 6B, left). After validating the predictive power of the 14-gene signature on the EMC286 dataset (FIG. 13A), which comprises early stage tumors from patients who did not undergo adjuvant chemotherapy or hormonal chemotherapy after surgery (Table 4b), its ability to predict overall metastatic relapse in a combined dataset comprising the MSK82, EMC192, and EMC286 cohorts was tested. This combined dataset comprise patient populations with distinct clinical, pathologic and treatment characteristics. Approximately three quarters of the cases were lymphnode negative at diagnosis and did not receive adjuvant chemotherapy after resection of the primary tumor. The remainder cases were lymph-node positive or received hormonal therapy and/or chemotherapy (Bos et al., 2009). The cumulative frequency of metastasis to bone (24.3%), lung (10.7%), liver (10%), and brain (6.6%) was similar to that generally observed (Table 4a). Interestingly, we found that the 14-gene signature was strongly associated with overall metastatic relapse in this large cohort (P=2.9E-4; n=560) (FIG. 6B, right).

Further distillation of the 14-gene signature led to the identification of 2 of its component genes, KIAA1199 and NDRG1, whose combined overexpression predicted overall relapse in the EMC286 dataset with efficiency similar to that of the 14-gene signature (FIG. 9B). Although both genes encode for proteins of unknown cellular function, prior studies have suggested that KIAA1199 is induced by Wnt/β-catenin signaling during colorectal adenomatous transformation (Sabates-Bellver et al., 2007) and NDRG1 is activated by HIF-1α, through inactivation of Myc-mediated repression, and by AP-1 (Ellen et al., 2008). Interestingly, expression of the 2 genes strongly correlated with poor prognosis also in the large cohort comprising the MSK82, EMC192, EMC286, and NKI295 datasets (P=2.9E-7; n=855) (FIG. 13B).

To examine if expression of Coco correlated specifically with metastasis to the lung, we compared the levels of Coco protein in lung and bone metastatic variants of MDA-MB231 cells (Zhang et al., 2009). LM2-4180 and LM2-4175 cell lines, which display enhanced tropism to the lung as a consequence of in vivo selection in the mouse, expressed levels of Coco proportional to their lung metastatic capacity (Minn et al., 2005). In contrast, Coco was not upregulated in the bone metastatic variants Bo-1833 and Bo-2287 (FIG. 6C). Q-PCR analysis indicated that none of 9 additional secreted BMP inhibitors had a similar pattern of upregulation in lung metastatic variants. For example, the expression of Noggin and Chordin-like 1 was confined to the metastatic cells lines, but it decreased as lung metastatic ability increased, suggesting that passage in vivo and selection for lung tropism led to the isolation of cells with increased levels of Coco but decreased levels of other BMP inhibitors (FIG. 12A).

To corroborate the hypothesis that expression of Coco underlies organ-specific metastasis to the lung, we examined if the 14-gene and 2-gene signatures were able to selectively predict lung metastasis. Preliminary analyses indicated that both signatures were able to predict relapse to the lung but not to the bone or brain in the MSK82, EMC192, and EMC286 datasets (FIG. 13C). To avoid the potentially confounding effect of patients with multi-site metastases, we examined the ability of the 14-gene and the 2-gene signature to predict organ-specific metastasis in the large combined dataset (MSK82+EMC192+EMC286) after exclusion of such patients. The results indicated that both signatures were strongly associated with lung but not bone or brain metastasis (FIGS. 6D and 6E). Notably, multivariate analysis indicated that both signatures were able to predict overall survival as well as lung metastasis independently of transcriptomic subtype (Table 6), tumor size, lymphnode positivity, ER status, HER2 status, pathological grade or expression of the NKI 70-gene poor survival signature (see Tables 7 and 8 in APPENDIX 3).

TABLE 6

| | Variable | p-value | HR (95% CI) |
|---|---|---|---|
| Metastasis-free | Basal | 0.57 | 1.39 (.044, 4.41) |
| Survival | ERBB2 | 0.24 | 1.95 (0.64, 5.99) |
| | LuminalA | 0.34 | 1.69 (0.57, 5.04) |
| | LuminalB | 0.46 | 1.51 (0.50, 4.50) |
| | 14-gene signature | <0.0001 | 3.92 (2.12, 7.24) |
| | Basal | 0.20 | 2.09 (0.68, 6.44) |
| | ERBB2 | 0..37 | 3.17 (1.07, 9.38) |
| | LuminalA | 0.34 | 1.69 (0.57, 5.04) |
| | LuminalB | 0.13 | 2.29 (0.79, 6.64) |
| | 2-gene signature | <0.0001 | 2.65 (1.62, 4.33) |
| Lung-metastasis- | Basal | 0.90 | 1.06 (0.40, 2.84) |
| free | ERBB2 | 0.30 | 1.64 (0.64, 4.18) |
| Survival | LuminalA | 0.55 | 1.32 (0.53, 3.30) |
| | LuminalB | 0.70 | 1.20 (0.48, 3.01) |
| | 14-gene signature | <0.0001 | 3.87 (2.22, 6.73) |
| | Basal | 0.25 | 1.74 (0.67, 4.52) |
| | ERBB2 | 0.027 | 2.76 (1.12, 6.79) |
| | LuminalA | 0.55 | 1.32 (0.53, 3.29) |
| | LuminalB | 0.14 | 1.93 (0.80, 4.68) |
| | 2-gene signature | 0.0002 | 2.28 (1.47, 3.55) |

Finally, although the original Coco signature comprising 56 genes displayed target organ-specificity similar to that of the previously described Lung Metastasis Signature (FIG. 13D) and was able to predict overall survival with similar efficiency (Table 9), the two signatures only shared 3 genes, consistent with the involvement of biologically distinct mechanisms (Minn et al., 2005). The patient classification concordance method was used to compare the performance of the Coco signatures to that of the previously described Lung Metastasis Signature (LMS) (Minn et al., 2005). DNA microarray data were normalized across platforms and a uniform method was used to generate the risk scores. The 56-gene Coco signature and the LMS share three genes (LAPTM5, KIAA1199, AND OLFML2A). KIAA1199 is a shared component of all Coco signatures and the LMS. The 56-gene Coco signature and the LMS have similar predictive power (p<0.0001; n=368.

TABLE 9
Performance of the Coco signatures and Lung Metastasis
Signature in the MSD82 and EMC286 combined dataset
using the patient classification concordance method.

| | | # of | | # of | |
|---|---|---|---|---|---|
| | # of | deceased | # of | deceased | |
| | patients | patients | patients | patients | |
| | with high | with high | with low | with high | Log-rank |
| | risk score | risk score | risk score | risk score | p-value |
| 2-gene Coco | 148 | 25 | 220 | 14 | 0.00032 |
| signature | | | | | |
| 14-gene | 184 | 29 | 184 | 10 | 0.00052 |
| Coco | | | | | |
| signature | | | | | |
| (contains 13 | | | | | |
| unique | | | | | |
| genes) | | | | | |
| 56-gene | 184 | 31 | 184 | 8 | 3.16e−05 |
| Coco | | | | | |
| signature | | | | | |
| (47 | | | | | |
| unique | | | | | |
| genes) | | | | | |
| LMS (54 | 184 | 31 | 184 | 8 | 1.5e−05 |
| unique | | | | | |
| genes) | | | | | |

To examine if NDRG1 and KIAA1199 predict lung metastasis as a consequence of a functional role that they play in this process, the expression of the two genes was silenced, either singly or in combination, in MDA-MB231 cells (FIG. 13E-G). Single silencing of NDRG1 or KIAA1199 did not inhibit lung colonization (FIGS. 6F, 6G, 13E, and 13F). However, simultaneous down regulation of both genes led to a significant inhibition of lung colonization (more than 80% inhibition at 10 weeks; FIGS. 6H and 13G). These observations suggest that NDRG1 and KIAA1199 are Coco-regulated genes involved in lung colonization.

### Example 11

### Coco is not Required for Colonization of the Bone or Brain

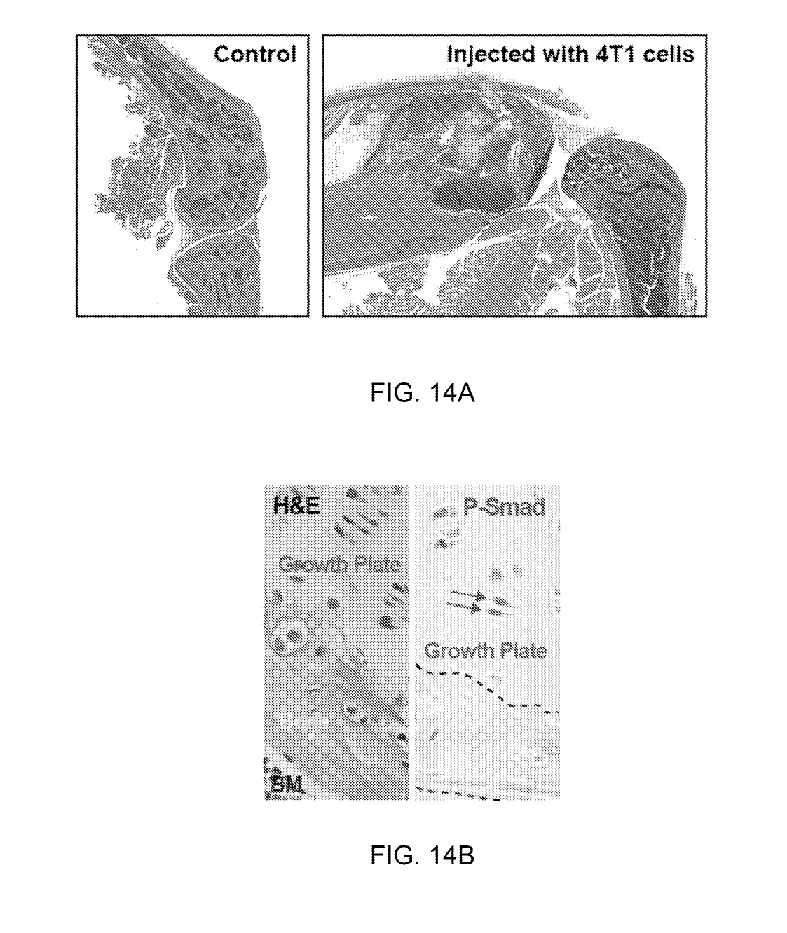

To directly assess if Coco participates in metastasis to the bone or brain, we injected control and Coco-silenced 4T1 cells in the left ventricle of syngeneic mice. Bioluminescent imaging indicated that a large fraction of mice injected with control cells developed bone, brain and adrenal gland metastases (FIGS. 7A and 7B). X-ray imaging as well as histological analysis confirmed that the 4T1 cells produce osteolytic bone lesions in more than 80% of the injected mice (FIGS. 7A and 14A). Notably, silencing of Coco did not reduce the rate of metastasis to bone, brain or adrenal gland and it did not inhibit the growth of individual lesions (FIGS. 7A and 7B), suggesting that Coco is not required for successful colonization of the bone, brain, or adrenal gland.

Early studies employing Northern blotting and in situ hybridization have suggested that the mRNAs encoding BMP3, BMP5, and BMP6 are highly enriched in the lung as compared to other organs of adult mice (King et al., 1994; Knittel et al., 1997; Vukicevic et al., 1994), suggesting that Coco may mediate organ-specific metastasis to the lung because the stroma of this organ contains particularly high levels of bioactive BMP proteins. Since the action of BMP proteins is regulated by multiple secreted inhibitors, some of which can neutralize each other or also function as direct activators, as well as by complex additional mechanisms (Walsh et al., 2010), it is impossible to estimate the amount of bioavailable BMP present in a given tissue by using direct methods. We therefore decided to gauge the amount of active BMP present in the bone marrow stroma by examining the activation of P-Smad signaling in tumor cells that had infiltrated this microenvironment. Preliminary analysis of bone sections from control mice indicated strong nuclear accumulation of BMP-responsive P-Smad proteins in chondrocytes in the growth plate (FIG. 14B). In contrast, although most hematopoietic cells stained weakly positive for anti-P-Smad 1, 5, 8, we noticed nuclear accumulation of the stain only in a minority of them (FIGS. 7C and 7D). Intriguingly, a large fraction of the solitary 4T1 cells that were detected in the bone marrow stroma 7 days after intracardiac injection did not display nuclear accumulation of BMP-responsive P-Smad proteins (FIG. 7C). Furthermore, virtually all the constituent cells in all micrometastases and osteolytic lesions detected at 5 weeks were similarly P-Smad-negative (FIG. 7D), suggesting that these outgrowths had originated from P-Smad-negative solitary tumor cells. Similar results were obtained after examining the micrometastatic lesions that arose in the bone of 2 out of 5 mice injected with 4TO 7 cells 7.5 weeks earlier (FIG. 7D). Finally, also all of the solitary tumor cells detected in the brain parenchyma of mice injected 7 days earlier with 4T1 cells did not display nuclear accumulation of BMP-responsive P-Smad proteins (FIG. 7C). In contrast, we had found that more than 94% of the solitary 4T1 cells and virtually all the solitary 4T07 that had seeded the lung after tail vein injection were P-Smad-positive (FIGS. 2H and 2J). These results suggest that the stromal microenvironments of the bone and brain contain lower levels of bioactive BMP proteins capable of engaging their cognate receptors on tumor cells as compared to those of the lung. Collectively, these results suggest that Coco mediates lung-specific colonization because it shields tumor cells from the inhibitory action of the particularly high levels of BMP proteins that they encounter upon infiltrating this organ.

### Example 12

### Materials and Methods

Cell Lines

The 67NR, 168FARN, 4T07, 66cl4 and 4T1 cell lines were generously provided by Dr. Fred R. Miller (Wayne State University, Detroit, Mich.) and cultured as described (Aslakson and Miller, 1992). To measure rates of cell growth, cells were seeded at 1×105 in 100 mm dishes and counted daily. The lung metastatic MDA-MB231 variants LM2-4180 and LM2-4175 and the bone-metastatic variants BoM-1833 and BoM-2287 and CN34.2a were a kind gift of Dr. Joan Massague (Cancer Biology and Genetics Program, MSKCC) and were cultured in DME-HG supplemented with 10% FBS. BT20, MDA-MB-415, MDA-MB-453, MDA-MB-157, and MDA-MB-231 cells (from ATCC) were cultured in the same medium. SK-Br3 cells (ATCC) were cultured in McCoys 5A supplemented with 10% FBS. BT474, T47D, ZR-751, HCC1428, and HCC1143 cell (ATCC) cells were cultured in RPMI 1640 supplemented with 10% FBS. The mouse ErbB2 cell line was setup in our lab as previously described (Guo et al., 2006). MDA-MB-468 cells (ATCC) were cultured in L15 supplemented with 10% FBS. For bioluminescent tracking, cell lines were infected with a retroviral vector encoding herpes simplex virus thymidine kinase 1, green fluorescent protein (GFP) and firefly luciferase (Ponomarev et al., 2004). GFP-positive cells were isolated by FACS.

### Antibodies

Immunoblotting experiments were performed with mouse Mabs to Myc-tag (9B11) form Cell Signaling, to β-actin (AC-74) and Flag-tag (M2) from Sigma, to GATA3 (HG3-31), laminin5-y2 chain (E-6), and SM-actin (B4) from Santa Cruz, to E [symbol] cadherin (36/E-cadherin), α-catenin (5/α-catenin), β-catenin (14/β-catenin), y [symbol] catenin (151y-catenin), fibronectin (10/fibronectin), vimentin (RV202), N-cadherin (32), and EpCAM (29) from BD Biosciences, to cytokeratin 8 (Troma-1) from Developmental Studies Hybridoma Bank, to ERa (AER611) from Thermo scientific, and to p63 (4A4) from NeoMarkers; with rabbit Mabs to Smad1 (EP565Y) and cytokeratin 18 (E431-1) from Abcam; with hamster Mab to Muc1 (MH1) from NeoMarkers; with goat antibodies to mouse Coco, human Coco, and CD10 from R&D and to cytokeratin 19 from Santa Cruz; with rabbit antibodies to P-Smad1,5,8 from Cell Signaling, to ZO-1 from Zymed, to PR, β-casein, ErbB2 and integrin β4 (H101) from Santa Cruz; with mouse antibodies to cytokeratin 14 and cytokeratin 5 from Covance, and with rabbit antibodies to NDRG1 from Invitrogen and to KIAA1199 from Abcam. Immunofluorescence experiments were conducted with mouse Mabs to PECAM-1 (MEC13.3), E-cadherin (36/E-cadherin), and β-catenin (14/β-catenin) from BD Biosciences, to GATA3 (HG3-31) from Santa Cruz; with rabbit Mab to cytokeratin 18 (E431-1) and chicken antibody to GFP from Abcam; with rat Mab to integrin β4 (346-11A) from BD Biosciences; with mouse antibodies to cytokeratin 14 from Covance; with goat antibodies to laminin5-β3 chain from Santa Cruz; with rabbit antibody to ZO-1 from Zymed. Antibodies used for immunohistochemistry included mouse Mabs MM1 to Ki-67 (Novocastra) and 14/β-catenin to β-catenin; rabbit antibodies to cleaved caspase 3 and P-Smad1,5,8 (Cell Signaling), and GFP (Abcam); and goat antibodies to human Coco (R&D). The CD24-PE (M1/69), CD49f-FITC (GoH3), CD24-PE (ML5), and CD44-APC (G44-26) antibodies used for FACS analysis were obtained from BD Bioscience, and the CD29-PE-Cy7 (HMβ1-1) was obtained from Biolegend.

Genome-Wide Retroviral cDNA Screen

The retroviral cDNA libraries were constructed essentially as described previously (Koh et al., 2004). Three high-complexity cDNA libraries were constructed from size-fractionated mRNA (3 pools: >3 Kb, 1-3 Kb, <1 Kb) isolated from 4T1 cells and cloned into the modified retroviral shuttle vector pEYK3.1, which uses the MoLV-LTR promoter to drive expression of N terminally Flag-tagged proteins (total complexity ˜2×10⁶ independent clones). The 67NR, 168FARN, or 4T07 cells were then infected independently with the three libraries at an MOI of 3:1 and injected subcutaneously in the mammary fat pad of BALB/c mice (1×10⁶ cells per #4 mammary gland). Macroscopic metastatic lesions were dissected and minced to isolate tumor cells. Clonogenic tumor cells were expanded in selective medium as described previously (Aslakson and Miller, 1992). To rescue proviral DNA, genomic DNA was isolated, digested with Ascl, re-ligated and transformed into Escherichia coli. Finally, proviral cDNA inserts from positive clones were analysed by DNA sequencing (Koh et al., 2002).

### Animal Studies

All mouse studies were conducted in accordance with protocols approved by the Institutional Animal Care and Use Committee of MSKCC.

67NR, 168FARN, 4T07, 66cl4 and 4T1 cells were transplanted in 6-12 weeks-old syngeneic BALB/c mice; MDA-MB231 cells were xenografted in 6-12 weeks-old nude BALB/c mice for tail vein injection or NOD/SCID/IL2Rynull mice for tumor initiation assay; ErbB2 cells were xenografted in 6-12 weeks-old nude BALB/c mice; and CN34.2a cells were xenografted in 6-12 weeks-old NOD/SCID/IL2Rynull mice. Orthotopic injections in the mammary fat pad and experimental metastasis assays were performed essentially as previously described (Guo et al., 2006; Pylayeva et al., 2009). To analyze spontaneous metastasis, 4T07 cells and their derivatives expressing cDNA1 or Coco cells were transduced with the TGL vector, resuspended at 1×10⁶ in 20 μl DME-HG supplemented with 10% FBS, and injected in the No. 4 mammary gland. Lung colonization was assessed by bioluminescent imaging at the indicated times. The size of primary tumors was measured 46 days following tumor implantation. To examine lung colonization, cells transduced with the TGL vector (Ponomarev et al., 2004) were harvested by trypsinization, washed twice in PBS, resuspended at 3×10⁵ (4T07 cells and their derivatives transduced with cDNA1, Coco, Smad6, or βcat-S4A), 1×10⁵ (4T1 cells and their derivatives transduced shCoco #1, shCoco #3, or CA-BMPR), or 5×10⁶ (ErbB2 cells and their derivatives transduced shCoco #1, shCoco #3), 1×10⁶ (66cl4 cells and their derivatives transduced shCoco #1, shCoco #3, and CN34.2a cells, MDA-MB-231 cells, and their derivatives transduced with shCoco #2 and shCoco #4) in 50 μl of PBS, and injected in the tail vein. To examine P-Smad 1,5,8 activity and colonization of the bone, brain, and adrenal gland, 4T1 cells and their derivatives were injected in the left ventricle of mice at 3×10⁴ in 100 μl of PBS. Bioluminescent imaging was used to verify successful injection and to monitor metastatic outgrowth. Metastatic lesions were confirmed by histological analysis.

For tumor initiation experiments, the indicated numbers of cells were suspended in 20 μl DME-HG with 10% FBS and injected in the No. 4 mammary gland. Primary tumor growth was monitored weekly by taking measurements of tumor length (L) and width (W). Tumor volume was calculated using the formula πLW2/6.

For EdU incorporation assay, we followed the protocol from Invitrogen (A10044). Briefly, 4T07-TGL cells stably expressing Coco or not were inoculated intravenously into syngeneic mice. Mice were sacrificed at day 7, 21, and 35, and each mouse was continually injected of EdU at 400 ug/day for 3 days before sacrifice. Lung sections were processed for immunofluorescent detection of GFP and Edu (010339, Invitrogen).

### Immunostaining

Immunostaining was performed as previously described (Guo et al., 2006; Pylayeva et al., 2009). Mice were sacrificed and perfused with PBS through the left ventricle. Lungs were inflated with ice-cold 4% paraformaldehyde through an intra [symbol] tracheal injection, fixed overnight, and embedded in OCT. Tumors were also fixed with 4% paraformaldehyde overnight and embedded in OCT. To examine extravasation and outgrowth in the lung, 50 μm-thick non-consecutive sections were cut on a Leica microtome, and immunoflurorescence was performed with anti-PECAM-1 by using the Tyramide Signal Amplification Kit (Invitrogen) to visualize lung capillaries and anti-GFP to visualize tumor cells, followed by detection with fluorescently conjugated secondary antibodies (Jackson Immunoresearch). Solitary tumor cells were randomly counted in 10 fields per section, and metastatic lesions were counted in whole sections (2-3 sections per mouse). We performed immunohistochemistry on paraffin-embedded sections by using the peroxidase-DAB or alkaline phosphatase-BCIP/NBT staining kits (MOM. and VECTASTAIN ABC) from Vector Laboratories. Immunohistochemical double staining for Ki-67, cleaved caspase-3, P-Smad1, 5, 8, β-catenin or GATA3 with GFP was performed on 8 μm-thick non-consecutive lung sections. Tumor cells were randomly counted in 10-30 fields per section (2-3 sections per mouse), or roughly 300-600 cells per lung (Ki-67). Human Coco immunohistochemical staining was performed using paraffin-embedded tissue microarrays (BioChain Institute, lmgenex and MSKCC breast center). Results were analysed by two people in a blind manner. The expression score was determined by combining staining intensity and the percentage of immunoreactive cells. Tissues with no staining were rated as 0, with a faint or moderate staining in less than 25% of cells as 0.5, with strong staining in less than 25% of cells or with moderate staining in 25-50% of cells as 1, with strong staining in 25-50% of cells or with moderate staining in more than 50% of cells as 1.5, with strong staining in more than 50% of cells as >2. Cells were fixed with 4% paraformaldehyde for immunofluorescent staining with antibodies to E-cadherin and β-catenin, whereas they were fixed with ice-cold methanol or acetone for immunofluorescent staining with antibodies to ZO-1, integrin β4, and laminin-5.

Bioluminescent and X-ray Imaging

For bioluminescent imaging, mice were anaesthetized and injected retro-orbitally with 1.5 mg of D-luciferin at the indicated times after xenografting. Animals were imaged in an IVIS 100 chamber within 5 minutes after D-luciferin injection, and data were recorded using Living Image software (Xenogen). To measure lung colonization, photon flux was calculated for each mouse by using a circular region of interest encompassing the thorax of the mouse. After subtracting a background value obtained from a control mouse injected only with D-luciferin, photon flux was normalized to the value obtained 30 minutes after injection of the tumor cells. This latter value was arbitrarily set at 100. To measure colonization of other organs after intracardiac injection, mice were imaged as described above except that values were not normalized after subtracting the background. Bone metastases were confirmed by X-Ray imaging. Mice were anesthetized by using a solution of Ketamine (100 mg/kg) and xylazine (10 mg/kg), placed on x-ray films (X-OMAT AR, Eastman Kodak, Rochester, N.Y.), and exposed at 25 kV for 15 s using a Faxitron instrument (Model MX-20; Faxitron Corp. Buffalo, Ill.).

### Tumor Sphere Assays

Mammosphere assays were performed as previously described (Dontu et al., 2003). Single-cell suspensions of 4T07 cells containing 1000 cells were plated on ultra-low attachment 6-well plates (Corning Costar) in serum-free mammary epithelial growth medium (MEBM, BioWhittaker) supplemented with 1:50 B27 (Invitrogen), 20 ng/mL EGF and 10 ng/mL bFGF (BD Biosciences), 4 μg/mL heparin, 4 μg/mL insulin, and 0.4% BSA (Sigma). Cells were cultured for 4-7 days and the tumorspheres with a diameter of >75 μm were counted. For serial passage, tumorspheres were collected using 70 μm cell strainers and dissociated with trypsin for 15 min to obtain single-cell suspensions.

Single cells suspension of ErbB2 and MDA-MB231 cells (1000 cells/ml) were plated on ultra low attachment plate for tumorsphere culture. Cells were cultured in serum free MEBM supplemented with 5□g/ml insulin, 0.5 □g/ml hydrocortisone, 1:50 B27, 20 ng/ml bFGF and EGF and 4□g/ml insulin heparin for 7 days. Tumor spheres were visualized under phase contrast microscope, photographed and counted and represented graphically.

PKH-26 staining was performed as described earlier (Cicalese et al, Cell 2009). ErbB2 cells were stained with PKH-26 dye (Sigma; 1:250 dilutions for 5 minutes) as manufactures instruction. The staining was stopped by adding 1% BSA to the cell suspension. PKH-26 positive cells were isolated by FACS and using for primary tumorsphere formation. PKH-26 positive and negative cells were sorted from primary tumor sphere and secondary tumor sphere and used for secondary and tertiary tumor sphere formation respectively.

### DNA Microarray Analysis

Extraction, labelling and hybridization of the samples to HG-U133A 2.0 gene expression chip (Affymetrix) were performed by the MSKCC Genomics Core Facility using standard methodology.

### Bioinformatics Analysis

DNA microarray results were analyzed by using Partek 6.4. Class comparison between triplicate MDA-MB-231-shControl, shCoco #2 and shCoco #4 samples was performed to identify gene expression changes of >=2-fold associated with Coco knockdown. As a result, we obtained a list of 56 probe sets, corresponding to 47 genes. This gene set was used to classify samples from human primary breast cancer data sets (MSK82, EMC192, EMC286, and NKI295) into groups whose gene expression patterns resembled Coco-silenced or control MDA-MB-231 cells. For each dataset, the gene expression values of each patient sample were normalized by subtracting the sample mean and then dividing by the sample standard deviation. Cox proportional hazards regression was used to evaluate whether genes were univariately associated with the metastasis-free survival (MFS) for the three data sets respectively. To determine whether gene expression profiles were associated with the MFS, we used one of the data sets as the training set, and then the remainder as the testing sets. Significant genes (p-value <0.05) in the training set were used to create a risk score. In addition, the genes NDRG1 and KIAA1199 had p-values less than 0.05 in all three datasets and were also used to create a risk score. The risk score was defined as a linear combination of the gene expression values of the significant genes weighted by their estimated regression coefficients (Beer et al., 2002). Patients were assigned to a low risk group if their risk index valued less than the 50th (for 14 gene signature) or 60th (for the 2 gene signature) percentile of the risk score values and to a high risk group otherwise. Kaplan-Meier survival curves and log rank tests were used to evaluate whether the assignment using the risk score values was validated in the test sets. Cox proportional hazards regression model was used for multivariate analysis. For the categorical variables, the term following the variable name denotes the group that is used to compare with the reference. For example, ER− follows ER, which means ER+ (not shown in the table) is the reference.

### Constructs

cDNAs encoding full-length mouse Coco, human BMPRII, mouse BMPRIB Q203D, mouse BMPRIB K231R, and Smad6 were obtained from Open Biosystems, Massague lab or Addgene and sub-cloned into pBabepuro or pQCXIP (Clontech) retroviral vectors. β-catenin S4A (S35/37/41/45A) (Okada et al., 2005) was also sub-cloned in pBabehygro. pLKO.1 vectors encoding short hairpin RNAs against mouse Coco (TRCN0000097620 and TRCN0000097621), human NDRG1 (TRCN0000084045 and TRCN0000084045), and human KIAA1199 (TRCN0000118787 and TRCN0000118791) as well as control vectors were obtained from Open Biosystems. pLKO.1 vectors encoding short hairpin RNAs against human Coco (TRCN0000149666 and TRCN0000148148) were obtained from Sigma.

For immunoblotting, cells were washed with PBS and lysed in RIPA buffer (50 mM Tris-HCl pH 7.4, 150 mM NaCl, 1 mM EDTA, 1% Triton X-100, 1% sodium deoxycholate, and 0.1% SDS) supplemented with 100 μM NaVO3 and protease inhibitors (Calbiochem). Protein concentrations were measured by using the DC Protein Assay. To quantify the amount of Coco secreted in the medium, 1.5×10⁶ cells were seeded in 100 mm plates and cultured in 4 ml of serum-free medium for 24 hours. Proteins that had accumulated in the conditioned medium were collected by TCA precipitation and subjected to immunoblotting.

Total RNA was extracted using RNeasy Mini kit coupled with RNase-free DNase set (Qiagen) and reverse transcribed with SuperScript III First-Strand Synthesis SuperMix (Invitrogen). cDNA corresponding to approximately 10 ng of starting RNA was used for one reaction. Q-PCR was performed with Taqman Gene Expression Assay (Applied Biosystems). All quantitations were normalized to endogenous GAPDH. Primers sequence used to amplify genes are listed as below.

ErbB2 cells were cultured in matrigel for 7 days with or without 100 ng/ml BMP4 (R&D). Cells were recovered from matrigel using Cell recovery solution (BD Bioscience). Total RNA was isolated from the cells and analyzed by Q-PCR.

| Primer | Primer | Primer | Primer |
|---|---|---|---|
| Name | Sequence | Name | Sequence |
| BMPRIA5 | atgcaaggattcaccgaaag | Cripto-5 | tgtgttctgggcagtttctg |
| BMPR-IA-3 | atagcggcctttaccaacct | Cripto-3 | ggcagtacagggctgagaag |
| BMPR-IB-5 | gtcgtgacactcccattcct | TGF6R- | ggcgaaggcattacagtgtt |
| | | I-5 | |
| BMPRIB3 | atagcggccttttccaatct | TGF6R- | tgcacatacaaatggcctgt |
| | | I-3 | |
| BMPRII5 | atggaacataccgcttttgc | TGF6R- | gcaagttttgcgatgtgaga |
| | | II-5 | |
| BMPRII3 | gtgagcctctcgtctccaac | TGF6R- | ggcatcttccagagtgaagc |
| | | II-3 | |
| ActRIA-5 | gtttcccacatcaagctggt | LRP5.5 | caggtgcttgtgtggagaga |
| ActRIA-3 | gtttcccacatcaagctggt | LRP5.3 | catgttggtgtccaggtcag |
| ActRIIA-5 | acacagcccacttcaaatcc | LRP6.5 | ggtgtcaaagaagcctctgc |
| ActRIIA-3 | ggtgcctcttttctctgcac | LRP6.3 | acctcaatgcgatttgttcc |
| ActRIIB-5 | catcatcacgtggaacgaac | BMP1.5 | cactccacagcaggaagtga |
| ActRIIB-3 | cttgtggacaaccacctcct | BMP1.3 | ctcagtgaaagctccggttc |
| ActR-IB-5 | aactgcttgcagtgggaagt | BMP2.5 | tggaagtggcccatttagag |
| ActR-IB-3 | ggaaacttccggttcctctc | BMP2-3 | tgacgcttttctcgtttgtg |
| ActR-IC-5 | tctggtctgcctctcttggt | BMP3-5 | tgctgtggctctatgacagg |
| ActR-10-3 | tctcgtgtctcagcatcacc | BMP3-3 | accccttcgtttgaggagtt |
| Frizzled | caaggtttacgggctcatgt | BMP4.5 | tgagcctttccagcaagttt |
| 1-5 | | | |
| Frizzled | gagaaagccagcgatgtagg | BMP4-3 | cttcccggtctcaggtatca |
| 1-3 | | | |
| Frizzled | ttagcggcctgagagatgtt | BMP5.5 | ttcaaggcaagcgaggtact |
| 2-5 | | | |
| Frizzled | caggagagacggttgagagc | BMP5-3 | aatgcagcatacccttctgg |
| 2-3 | | | |
| Frizzled | tgggttggaagcaaaaagac | BMP6.5 | ttcttcaaggtgagcgaggt |
| 3-5 | | | |
| Frizzled | cctgctttgcttctttggtc | BMP6-3 | tagttggcagcgtagccttt |
| 3-3 | | | |
| Frizzled | ctgcagcatgcctaatgaga | BMP7.5 | gaaaacagcagcagtgacca |
| 4-5 | | | |
| Frizzled | cgtctgcctagatgcaatca | BMP7-3 | ggtggcgttcatgtaggagt |
| 4-3 | | | |

TaqMan Gene Expression Assays

| Gene Name | Assay ID |
|---|---|
| Cerberus 1 | Hs00193796_m1 |
| Chordin | Hs00415315_m1 |
| Chordin-like 1 | Hs00292767_m1 |
| Chordin-like 2 | Hs00248808_m1 |
| DAN | Hs00185054_m1 |
| Coco | Hs00541488_m1 |
| Gremlin 1 | Hs00171951_m1 |
| Gremlin 2 | Hs00254699_m1 |
| Noggin | Hs00271352_s1 |
| SOSTDC1 | Hs00383602_m1 |
| GAPDH | Hs99999905_m1 |

### Xenopus Studies

Transcription assays for BMP, Nodal, and Wnt signalling were performed as previously described (Hata et al., 2002; Liu et al., 1997; van de Wetering et al., 1997). 4-cell stage embryos were injected in the animal pole with 25 pg of reporter gene DNA fused to luciferase (Bmp response element, BRE; nodal/activin response element, A3Luc; Wnt responsive element, TOP-FLASH) in combination with RNA encoding ligand (100 pg Bmp4 RNA, 30 pg Nodal RNA, or 10 pg Wnt8 RNA) with or without the indicated amounts of Dante RNA. Embryos were recovered at stage 10 and subjected to transcription assays using the Luciferase Assay (Promega) as described (Vonica and Brivanlou, 2007).

### Invasion Assays

Invasion assays were performed as previously described (Guo et al., 2006; Pylayeva et al., 2009). Cells (1×10⁵) were placed on Transwell inserts coated with 8 μg Matrigel (BD Biosciences) in SFM. After incubation in wells containing SFM+10% FBS for 24 hrs, the inserts were fixed with 4% paraformaldehyde and stained with crystal violet. Experiments were performed in triplicate.

### Luciferase Reporter Assays

Cells (1×10⁵) were plated in 24-well dishes. For Nodal transcription assay, 200 ng Nodal reporter gene construct and 3 ng Renilla luciferase vector were co [symbol] transfected per well. Nodal was added after 24 hrs, and cell extracts were prepared 48 hrs later. For Wnt transcription assay, 200 ng Wnt reporter gene construct, Wnt1 and 3 ng Renilla luciferase vector were co-transfected per well. Cell extracts were prepared 48 hr after transfection. The luciferase activity was measured using the Dual-Luciferase Reporter Assay System (Promega). All experiments were performed in triplicate.

### FACS Analysis

4T07 cells were fixed in 4% formaldehyde for 10 min at 37° C. and permeabilized with ice-cold methanol for 30 min. Non-specific binding sites were saturated with rat IgGs (Sigma) and Armenian Hamster IgGs (Biolegend) for 10 min. Antibody incubations were performed at 4° C. overnight. Cell sorting was carried out on a FACS Vantage cell sorter (Becton Dickinson).

MDA-MB-231 cells were detached from culture plate using Accutase (Innovative Cell Tec) and washed with cold HBSS supplemented with 5% FBS. Antibody incubation was performed at room temperature for 45 minutes. Cell sorting was carried out on a FACS Vantage cell sorter (Becton Dickinson).

### Propidium Iodide (PI) Staining and Cell Cycle Analysis

4T07 and ErbB2 cells were plated on ultra low attachment plate for and maintained for three days in tumorsphere formation medium. After 3 days single cells suspension were prepared using Accutase (Innovative Cell Tec), wash in PBS and then fixed in 70% ethanol. Cells were incubated in 2 mg/ml RNAase A and 0.1 mg/ml PI (Sigma) for 45 minutes in room temperature, washed and then analyzed by flow cytometry.

### MTT Assay

1×10³ MDA-MB-231 cells (Scrambled or Knockdown for Coco) were plated on each well of 96 well for 24 hour. After 24 Hour cells were incubated in 0.5 mh/ml MTT (3-{4,5-dimethylthiazol-2-yl}-2,5-diphenyl tetrazolium bromide; Invitrogen) for 1 h at 370 c. MTT crystals were dissolved in isopropanol and absorbance was measured in a plate reader at 570 nM.

### BrdU Incorporation Assay

MDA-MB-231 cells were seeded on coverslip for 24 hour followed by incubated with BrdU for another 12 h. Cells were fixed with 100% cold methanol and stained with BrdU labeling and detection kit (Roche), visualized under fluorescence microscope and counted.

### Colony Formation Assays

To measure clonogenicity, cells were plated at clonal density (1000 cells/100 mm plate) under standard culture conditions. Holoclones were counted 8 days later. For soft agar assays, 10⁴ cells were suspended in 0.3% agar in normal growth medium supplemented with 10% FBS and plated in triplicate over a layer of 0.6% agar base medium in 6-well plates. After 8 days, colonies were stained with 0.05% crystal violet and counted.

### Human Metastasis Samples

Paraffin-embedded tissue microarray sections with multiple primary breast tumor cores were from the MSKCC Department of Pathology in compliance with protocols approved by the MSKCC Institutional Review Board and after the subjects gave their informed consent. Coco expression was determined using immunohistochemistry. Total Coco immunoreactivity was evaluated and scored by a clinical pathologist (E.B.). We analyzed overall survival as a function of Coco abundance in breast tumor samples.

### Statistical Analysis

Results are reported as mean±SD or ±SEM unless otherwise noted. Comparisons between two groups were performed using an unpaired two-sided t test (p<0.05 was considered significant).

### Accession Numbers

Gene expression data of MDA231-sh-control, sh-Coco #2, and sh-Coco #4 cells are deposited at Gene Expression Omnibus (GSE28049).

### REFERENCES

Aguirre-Ghiso, J. A. (2007). Models, mechanisms and clinical evidence for cancer dormancy. Nat Rev Cancer 7, 834-846.
Al-Hajj, M., Wicha, M. S., Benito-Hernandez, A., Morrison, S. J., and Clarke, M. F. (2003). Prospective identification of tumorigenic breast cancer cells. Proceedings of the National Academy of Sciences of the United States of America 100, 3983-3988.
Aslakson, C. J., and Miller, F. R. (1992). Selective events in the metastatic process defined by analysis of the sequential dissemination of subpopulations of a mouse mammary tumor. Cancer Res 52, 1399-1405.
Bell, E., Munoz-Sanjuan, I., Altmann, C. R., Vonica, A., and Brivanlou, A. H. (2003). Cell fate specification and competence by Coco, a maternal BMP, TGFbeta and Wnt inhibitor. Development 130, 1381-1389.
Ben-Porath, I., Thomson, M. W., Carey, V. J., Ge, R., Bell, G. W., Regev, A., and Weinberg, R. A. (2008). An embryonic stem cell-like gene expression signature in poorly differentiated aggressive human tumors. Nature genetics 40, 499-507.
Blick, T., Hugo, H., Widodo, E., Waltham, M., Pinto, C., Mani, S. A., Weinberg, R. A., Neve, R. M., Lenburg, M. E., and Thompson, E. W. (2010). Epithelial mesenchymal transition traits in human breast cancer cell lines parallel the CD44(hi/)CD24 (lo/-) stem cell phenotype in human breast cancer. J Mammary Gland Biol Neoplasia 15, 235-252.
Bos, P. D., Zhang, X. H., Nadal, C., Shu, W., Gomis, R. R., Nguyen, D. X., Minn, A. J., van de Vijver, M. J., Gerald, W. L., Foekens, J. A., et al. (2009). Genes that mediate breast cancer metastasis to the brain. Nature 459, 1005-1009.
Brabletz, T., Jung, A., Spaderna, S., Hlubek, F., and Kirchner, T. (2005). Opinion: migrating cancer stem cells—an integrated concept of malignant tumour progression. Nat Rev Cancer 5, 744-749.
Buijs, J. T., Henriquez, N. V., van Overveld, P. G., van der Horst, G., Que, I., Schwaninger, R., Rentsch, C., Ten Dijke, P., Cleton-Jansen, A. M., Driouch, K., et al. (2007). Bone morphogenetic protein 7 in the development and treatment of bone metastases from breast cancer. Cancer research 67, 8742-8751.
Buijs, J. T., van der Horst, G., van den Hoogen, C., Cheung, H., de Rooij, B., Kroon, J., Petersen, M., van Overveld, P. G., Pelger, R. C., and van der Pluijm, G. (2011). The BMP2/7 heterodimer inhibits the human breast cancer stem cell subpopulation and bone metastases formation. Oncogene.
Cameron, M. D., Schmidt, E. E., Kerkvliet, N., Nadkarni, K. V., Morris, V. L., Groom, A. C., Chambers, A. F., and MacDonald, I. C. (2000). Temporal progression of metastasis in lung: cell survival, dormancy, and location dependence of metastatic inefficiency. Cancer research 60, 2541-2546.
Chen, Q., Zhang, X. H., and Massague, J. (2011). Macrophage binding to receptor VCAM-1 transmits survival signals in breast cancer cells that invade the lungs. Cancer Cell 20, 538-549.
Chi, S. W., Zang, J. B., Mele, A. and Darnell, R. B. (2009). Ago HITS-CLIP decodes miRNA-mRNA interaction maps. Nature 460(7254), 479-486.
Cicalese, A., Bonizzi, G., Pasi, C. E., Faretta, M., Ronzoni, S., Giulini, B., Brisken, C., Minucci, S., Di Fiore, P. P., and Pelicci, P. G. (2009). The tumor suppressor p53 regulates polarity of self-renewing divisions in mammary stem cells. Cell 138, 1083-1095.
Clevers, H. (2011). The cancer stem cell: premises, promises and challenges. Nature medicine 17, 313-319.
Cordenonsi, M., Zanconato, F., Azzolin, L., Forcato, M., Rosato, A., Frasson, C., Inui, M., Montagner, M., Parenti, A. R., Poletti, A., et al. (2011). The Hippo transducer TAZ confers cancer stem cell-related traits on breast cancer cells. Cell 147, 759-772.
Dankort, D., Maslikowski, B., Warner, N., Kanno, N., Kim, H., Wang, Z., Moran, M. F., Oshima, R. G., Cardiff, R. D., and Muller, W. J. (2001). Grb2 and Shc adapter proteins play distinct roles in Neu (ErbB-2)-induced mammary tumorigenesis: implications for human breast cancer. Mol Cell Biol 21, 1540-1551.
De Robertis, E. M., and Kuroda, H. (2004). Dorsal-ventral patterning and neural induction in Xenopus embryos. Annu Rev Cell Dev Biol 20, 285-308.
Dontu, G., Abdallah, W. M., Foley, J. M., Jackson, K. W., Clarke, M. F., Kawamura, M. J., and Wicha, M. S. (2003). In vitro propagation and transcriptional profiling of human mammary stem/progenitor cells. Genes Dev 17, 1253-1270.
Ellen, T. P., Ke, Q., Zhang, P., and Costa, M. (2008). NDRG1, a growth and cancer related gene: regulation of gene expression and function in normal and disease states. Carcinogenesis 29, 2-8.
Fidler, I. J. (2003). The pathogenesis of cancer metastasis: the ‘seed and soil’ hypothesis revisited. Nat Rev Cancer 3, 453-458.
Gonczy, P. 2008. Mechanisms of asymmetric cell division: flies and worms pave the way. Nat Rev Mol Cell Biol 9:355-366.
Guo, W., Pylayeva, Y., Pepe, A., Yoshioka, T., Muller, W. J., Inghirami, G., and Giancotti, F. G. (2006). Beta 4 integrin amplifies ErbB2 signaling to promote mammary tumorigenesis. Cell 126, 489-502.
Gupta, G. P., Nguyen, D. X., Chiang, A. C., Bos, P. D., Kim, J. Y., Nadal, C., Gomis, R. R., Manova-Todorova, K., and Massague, J. (2007a). Mediators of vascular remodelling co-opted for sequential steps in lung metastasis. Nature 446, 765-770.
Gupta, G. P., Perk, J., Acharyya, S., de Candia, P., Mittal, V., Todorova-Manova, K., Gerald, W. L., Brogi, E., Benezra, R., and Massague, J. (2007b). ID genes mediate tumor reinitiation during breast cancer lung metastasis. Proceedings of the National Academy of Sciences of the United States of America 104, 19506-19511.
Gupta, P. B., Chaffer, C. L., and Weinberg, R. A. (2009). Cancer stem cells: mirage or reality? Nat Med 15, 1010-1012.
Hata, A., Lagna, G., Massague, J., and Hemmati-Brivanlou, A. (1998). Smad6 inhibits BMP/Smad1 signaling by specifically competing with the Smad4 tumor suppressor. Genes Dev 12, 186-197.
He, X. C., Zhang, J., Tong, W. G., Tawfik, 0., Ross, J., Scoville, D. H., Tian, Q., Zeng, X., He, X., Wiedemann, L. M., et al. (2004). BMP signaling inhibits intestinal stem cell self-renewal through suppression of Wnt-beta-catenin signaling. Nat Genet 36, 1117-1121.
Hermann, P. C., Huber, S. L., Herrler, T., Aicher, A., Ellwart, J. W., Guba, M., Bruns, C. J., and Heeschen, C. (2007). Distinct populations of cancer stem cells determine tumor growth and metastatic activity in human pancreatic cancer. Cell Stem Cell 1, 313-323.
Husemann, Y., Geigl, J. B., Schubert, F., Musiani, P., Meyer, M., Burghart, E., Forni, G., Eils, R., Fehm, T., Riethmuller, G., et al. (2008). Systemic spread is an early step in breast cancer. Cancer Cell 13, 58-68.
Izzi, L., and Attisano, L. 2004. Regulation of the TGFbeta signalling pathway by ubiquitin-mediated degradation. Oncogene 23:2071-2078.
Karrison, T. G., Ferguson, D. J., and Meier, P. (1999). Dormancy of mammary carcinoma after mastectomy. J Natl Cancer Inst 91, 80-85.
King, J. A., Marker, P. C., Seung, K. J., and Kingsley, D. M. (1994). BMP5 and the molecular, skeletal, and soft-tissue alterations in short ear mice. Developmental biology 166, 112-122.
Knittel, T., Fellmer, P., Muller, L., and Ramadori, G. (1997). Bone morphogenetic protein-6 is expressed in nonparenchymal liver cells and upregulated by transforming growth factor-beta 1. Exp Cell Res 232, 263-269.
Kobielak, K., Stokes, N., de la Cruz, J., Polak, L., and Fuchs, E. (2007). Loss of a quiescent niche but not follicle stem cells in the absence of bone morphogenetic protein signaling. Proc Natl Acad Sci USA 104, 10063-10068.
Koh, E. Y., Chen, T., and Daley, G. Q. (2002). Novel retroviral vectors to facilitate expression screens in mammalian cells. Nucleic Acids Res 30, e142.
Koh, E. Y., Chen, T., and Daley, G. Q. (2004). Genetic complementation of cytokine signaling identifies central role of kinases in hematopoietic cell proliferation. Oncogene 23, 1214-1220.
Korpal, M., Ell, B. J., Buffa, F. M., Ibrahim, T., Blanco, M. A., Celia-Terrassa, T., Mercatali, L., Khan, Z., Goodarzi, H., Hua, Y., et al. (2011). Direct targeting of Sec23a by miR-200s influences cancer cell secretome and promotes metastatic colonization. Nature medicine 17, 1101-1108.
Kouros-Mehr, H., Bechis, S. K., Slorach, E. M., Littlepage, L. E., Egeblad, M., Ewald, A. J., Pai, S. Y., Ho, I. C., and Werb, Z. (2008). GATA-3 links tumor differentiation and dissemination in a luminal breast cancer model. Cancer Cell 13, 141-152.
Lacroix, M., and Leclercq, G. (2004). Relevance of breast cancer cell lines as models for breast tumours: an update. Breast Cancer Res Treat 83, 249-289.
Lim, D. A., Tramontin, A. D., Trevejo, J. M., Herrera, D. G., Garcia-Verdugo, J. M., and Alvarez-Buylla, A. (2000). Noggin antagonizes BMP signaling to create a niche for adult neurogenesis. Neuron 28, 713-726.
Liu, J. C., Deng, T., Lehal, R. S., Kim, J., and Zacksenhaus, E. (2007). Identification of tumorsphere- and tumor-initiating cells in HER2/Neu-induced mammary tumors. Cancer research 67, 8671-8681.
Lo, P. K., Kanojia, D., Liu, X., Singh, U. P., Berger, F. G., Wang, Q., and Chen, H. (2011). CD49f and CD61 identify Her2/neu-induced mammary tumor-initiating cells that are potentially derived from luminal progenitors and maintained by the integrin-TGFbeta signaling. Oncogene.
Lu, X., Mu, E., Wei, Y., Riethdorf, S., Yang, Q., Yuan, M., Yan, J., Hua, Y., Tiede, B. J., Haffty, B. G., et al. (2011). VCAM-1 Promotes Osteolytic Expansion of Indolent Bone Micrometastasis of Breast Cancer by Engaging alpha4beta1-Positive Osteoclast Progenitors. Cancer Cell 20, 701-714.
Luzzi, K. J., MacDonald, I. C., Schmidt, E. E., Kerkvliet, N., Morris, V. L., Chambers, A. F., and Groom, A. G. (1998). Multistep nature of metastatic inefficiency: dormancy of solitary cells after successful extravasation and limited survival of early micrometastases. Am J Pathol 153, 865-873.
Malanchi, I., Santamaria-Martinez, A., Susanto, E., Peng, H., Lehr, H. A., Delaloye, J. F., and Huelsken, J. (2012). Interactions between cancer stem cells and their niche govern metastatic colonization. Nature 481, 85-89.
Mani, S. A., Guo, W., Liao, M. J., Eaton, E. N., Ayyanan, A., Zhou, A. Y., Brooks, M., Reinhard, F., Zhang, C. C., Shipitsin, M., et al. (2008). The epithelial-mesenchymal transition generates cells with properties of stem cells. Cell 133, 704-715.
Mavrakis, K. J., Wolfe, A. L., Oricchio, E., Palomero, T., de Keersmaecker, K., McJunkin, K., Zuber, J., James, T., Khan, A. A., Leslie, C. S., et al. 2010. Genome-wide RNA-mediated interference screen identifies miR-19 targets in Notch-induced T-cell acute lymphoblastic leukaemia. Nature cell biology 12:372-379.
Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436, 518-524.
Neve, R. M., Chin, K., Fridlyand, J., Yeh, J., Baehner, F. L., Fevr, T., Clark, L., Bayani, N., Coppe, J. P., Tong, F., et al. (2006). A collection of breast cancer cell lines for the study of functionally distinct cancer subtypes. Cancer Cell 10, 515-527.
Nguyen, D. X., Bos, P. D., and Massague, J. (2009). Metastasis: from dissemination to organ-specific colonization. Nature reviews Cancer 9, 274-284.
Oricchio, E., Nanjangud, G., Wolfe, A. L., Schatz, J. H., Mavrakis, K. J., Jiang, M., Liu, X., Bruno, J., Heguy, A., Olshen, A. B., et al. 2011. The eph-receptor a7 is a soluble tumor suppressor for follicular lymphoma. Cell 147:554-564.
Oskarsson, T., Acharyya, S., Zhang, X. H., Vanharanta, S., Tavazoie, S. F., Morris, P. G., Downey, R. J., Manova-Todorova, K., Brogi, E., and Massague, J. (2011). Breast cancer cells produce tenascin C as a metastatic niche component to colonize the lungs. Nature medicine 17, 867-874.
Owens, P., Pickup, M. W., Novitskiy, S. V., Chytil, A., Gorska, A. E., Aakre, M. E., West, J., and Moses, H. L. (2011). Disruption of bone morphogenetic protein receptor 2 (BMPR2) in mammary tumors promotes metastases through cell autonomous and paracrine mediators. Proceedings of the National Academy of Sciences of the United States of America.
Padua, D., Zhang, X. H., Wang, Q., Nadal, C., Gerald, W. L., Gomis, R. R., and Massague, J. (2008). TGFbeta primes breast tumors for lung metastasis seeding through angiopoietin-like 4. Cell 133, 66-77.
Pang, R., Law, W. L., Chu, A. C., Poon, J. T., Lam, C. S., Chow, A. K., Ng, L., Cheung, L. W., Lan, X. R., Lan, F L Y., et al. (2010). A subpopulation of CD26+ cancer stem cells with metastatic capacity in human colorectal cancer. Cell Stem Cell 6, 603-615.
Pantel, K., Brakenhoff, R. H., and Brandt, B. (2008). Detection, clinical relevance and specific biological properties of disseminating tumour cells. Nat Rev Cancer 8, 329-340.
Pearce, J. J., Penny, G., and Rossant, J. (1999). A mouse cerberus/Dan-related gene family. Dev Biol 209, 98-110.
Podsypanina, K., Du, Y. C., Jechlinger, M., Beverly, L. J., Hambardzumyan, D., and Varmus, H. (2008). Seeding and propagation of untransformed mouse mammary cells in the lung. Science 321, 1841-1844.
Sabates-Bellver, J., Van der Flier, L. G., de Palo, M., Cattaneo, E., Maake, C., Rehrauer, H., Laczko, E., Kurowski, M. A., Bujnicki, J. M., Menigatti, M., et al. (2007). Transcriptome profile of human colorectal adenomas. Mol Cancer Res 5, 1263-1275.
Scheel, C., Eaton, E. N., Li, S. H., Chaffer, C. L., Reinhardt, F., Kah, K. J., Bell, G., Guo, W., Rubin, J., Richardson, A. L., et al. (2011). Paracrine and autocrine signals induce and maintain mesenchymal and stem cell states in the breast. Cell 145, 926-940.
Shackleton, M., Quintana, E., Fearon, E. R., and Morrison, S. J. (2009). Heterogeneity in cancer: cancer stem cells versus clonal evolution. Cell 138, 822-829.
Shibue, T., and Weinberg, R. A. (2009). Integrin beta1-focal adhesion kinase signaling directs the proliferation of metastatic cancer cells disseminated in the lungs. Proc Natl Acad Sci USA 106, 10290-10295.
Shimono, Y., Zabala, M., Cho, R. W., Lobo, N., Dalerba, P., Qian, D., Diehn, M., Liu, H., Panula, S. P., Chiao, E., et al. (2009). Downregulation of miRNA-200c links breast cancer stem cells with normal stem cells. Cell 138, 592-603.
Silva, J. M., Li, M. Z., Chang, K., Ge, W., Golding, M. C., Rickles, R. J., Siolas, D., Hu, G., Paddison, P. J., Schlabach, M. R., et al. 2005. Second-generation shRNA libraries covering the mouse and human genomes. Nature genetics 37:1281-1288.
Spagnoli, F. M., and Hemmati-Brivanlou, A. (2006). Guiding embryonic stem cells towards differentiation: lessons from molecular embryology. Curr Opin Genet Dev 16, 469-475.
Tavazoie, S. F., Alarcon, C., Oskarsson, T., Padua, D., Wang, Q., Bos, P. D., Gerald, W. L., and Massague, J. (2008). Endogenous human microRNAs that suppress breast cancer metastasis. Nature 451, 147-152.
Valastyan, S., and Weinberg, R. A. (2011). Tumor metastasis: molecular insights and evolving paradigms. Cell 147, 275-292.
Valastyan, S., Reinhardt, F., Benaich, N., Calogrias, D., Szasz, A. M., Wang, Z. C., Brock, J. E., Richardson, A. L., and Weinberg, R. A. 2009. A pleiotropically acting microRNA, miR-31, inhibits breast cancer metastasis. Cell 137:1032-1046.
van de Vijver, M. J., He, Y. D., van′t Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.
Vukicevic, S., Helder, M. N., and Luyten, F. P. (1994). Developing human lung and kidney are major sites for synthesis of bone morphogenetic protein-3 (osteogenin). J Histochem Cytochem 42, 869-875.
Walsh, D. W., Godson, C., Brazil, D. P., and Martin, F. (2010). Extracellular BMP-antagonist regulation in development and disease: tied up in knots. Trends Cell Biol 20, 244-256.
Wang, Y., Klijn, J. G., Zhang, Y., Sieuwerts, A. M., Look, M. P., Yang, F., Talantov, D., Timmermans, M., Meijer-van Gelder, M. E., Yu, J., et al. (2005). Gene-expression profiles to predict distant metastasis of lymph-node-negative primary breast cancer. Lancet 365, 671-679.
Weinberg, R. A. (2008). The many faces of tumor dormancy. APMIS 116, 548-551.
Wong, C. W., Lee, A., Shientag, L., Yu, J., Dong, Y., Kao, G., Al-Mehdi, A. B., Bernhard, E. J., and Muschel, R. J. (2001). Apoptosis: an early event in metastatic inefficiency. Cancer research 61, 333-338.
Wong, D. J., Liu, H., Ridky, T. W., Cassarino, D., Segal, E., and Chang, H. Y. (2008). Module map of stem cell genes guides creation of epithelial cancer stem cells. Cell Stem Cell 2, 333-344.
Yin, J. J., Mohammad, K. S., Kakonen, S. M., Harris, S., Wu-Wong, J. R., Wessale, J. L., Padley, R. J., Garrett, I. R., Chirgwin, J. M., and Guise, T. A. (2003). A causal role for endothelin-1 in the pathogenesis of osteoblastic bone metastases. Proc Natl Acad Sci USA 100, 10954-10959.
Young, R. A. (2011). Control of the embryonic stem cell state. Cell 144, 940-954.
Zhang, X. H., Wang, Q., Gerald, W., Hudis, C. A., Norton, L., Smid, M., Foekens, J. A., and Massague, J. (2009). Latent bone metastasis in breast cancer tied to Src-dependent survival signals. Cancer Cell 16, 67-78.

1. A gene expression panel or array comprising nucleic acids capable of hybridizing to an expression product of each of genes KIAA1199 and NDRG1.

2. The gene expression panel or array of claim 1 further comprising nucleic acids capable of hybridizing to an expression product of a gene selected from the group consisting of ALDH6A1, PTDSS1, SCD, MYO5C, TFPI, PNMA2, TMEM45A, PLAT, HSD17B10, PLAT, EVI2B, and JAK1.

3. The gene expression panel or array of claim 1, wherein said expression product or said nucleic acids capable of hybridizing to said expression product comprises a detectable label.

4. The gene expression panel or array of claim 1, wherein said nucleic acid is from about 10 to about 80 nucleotides in length.

5. The gene expression panel or array of claim 1, wherein said nucleic acid is from about 20 to about 70 nucleotides in length.

6. The gene expression panel or array of claim 1 further comprising a nucleic acid capable of hybridizing to an expression product of a housekeeping gene.

7. The gene expression panel or array of claim 7, wherein said housekeeping gene is GAPDH.

8. A method of predicting the likelihood of metastasis-free survival of a breast cancer patient, comprising
(a) contacting a gene expression product from a breast cancer cell from the patient with a gene expression panel or array comprising nucleic acids capable of hybridizing to an expression product of each of genes KIAA1199 and NDRG1 to determine the expression level of KIAA1199 and NDRG1 in said breast cancer cell from said patient;
(b) comparing the expression level in said breast cancer cell from the patient with a reference expression level of genes characteristic of metastatic breast cancer cells,
wherein expression levels of KIAA1199 and NDRG1 that are equal to or greater than the reference expression level of said genes indicates a decreased likelihood of overall survival.

9. The method of claim 8, wherein said gene expression panel or array comprises nucleic acids capable of hybridizing to an expression product of each of genes selected from the group consisting of ALDH6A1, PTDSS1, SCD, MYO5C, TFPI, PNMA2, TMEM45A, PLAT, HSD17B10, PLAT, EVI2B, and JAK1.

10. The method of claim 8, wherein said expression product is RNA or cDNA.

11. The method of claim 8, wherein said expression product or nucleic acids capable of hybridizing to said expression product comprises a detectable label.

12. The method of claim 8, wherein said nucleic acid is from about 10 to about 80 nucleotides in length.

13. The method of claim 8, wherein said nucleic acid is from about 20 to about 70 nucleotides in length.

14. The method of claim 8, wherein the expression level of a gene product of each of genes tested is normalized against the expression level of all expression products in said breast cancer cell, or of a reference set of expression products.

15. The method of claim 8, wherein said expression product is isolated from a fixed, embedded breast cancer tissue specimen, a core biopsy of cancer tissue or fine needle aspirate of breast cancer cells of said patient.

16. A method of predicting the metastatic capacity of an isolated breast cancer cell, the method comprising:
(a) contacting a sample containing a gene expression product from the isolated breast cancer cell with a gene expression panel or array comprising nucleic acids capable of hybridizing to an expression product of each of genes KIAA1199 and NDRG1 to determine the expression level of KIAA1199 and NDRG1 in said isolated breast cancer cell;
(b) comparing said expression level in said isolated breast cancer cell with a reference expression level of said genes characteristic of metastatic breast cancer cells,
wherein expression of KIAA1199 and NDRG1 in said isolated breast cancer cell that is equal to or greater than the reference expression level indicates that the isolated breast cancer cell has metastatic capacity.

17. The method of claim 16, further comprising contacting said sample containing a gene expression product from the isolated breast cancer cell with a gene expression panel or array comprising nucleic acids capable of hybridizing to an expression product of a gene selected from the group consisting of ALDH6A1, PTDSS1, SCD, MYO5C, TFPI, PNMA2, TMEM45A, PLAT, HSD17B10, PLAT, EVI2B, and JAK1.

18. The method of claim 16, further comprising determining the expression level of from 2-12 genes selected from the group consisting of ALDH6A1, PTDSS1, SCD, MYO5C, TFPI, PNMA2, TMEM45A, PLAT, HSD17B10, PLAT, EVI2B, and JAK1.

19. The method of claim 16, wherein genes ALDH6A1, PTDSS1, SCD, MYO5C, TFPI, PNMA2, TMEM45A, PLAT, PLAT, EVI2B, and JAK1 are upregulated in metastatic breast cancer and HSD17B10 is downregulated.

20. A method for predicting the likelihood of relapse in a breast cancer patient, said method comprising:
a. contacting a sample containing a gene expression product from an isolated breast cancer cell or tissue from the patient with a gene expression panel or array comprising nucleic acids capable of hybridizing to an nucleic acid expression product of Coco or an anti-Coco antibody capable of binding a protein expression product of Coco;
b. comparing the level of expression product in said sample with the level of Coco expression in normal tissue,
wherein increased expression of Coco positively correlates with increased likelihood of breast cancer relapse in said patient.

21. A method of suppressing the metastatic capacity of breast cancer cells, said method comprising contacting said breast cancer cells with a therapeutic agent that reduces the expression and/or activity of Coco in said cells.

22. A method for reducing the risk of relapse and/or metastasis to the lung in a breast cancer patient comprising:
a) contacting a sample containing a gene expression product from a isolated breast cancer cell from the patient with a gene expression panel or array comprising nucleic acids capable of hybridizing to an expression product of each of genes KIAA1199 and NDRG1 to determine the expression level of KIAA1199 and NDRG1 in said isolated breast cancer cell;
b) comparing said expression level of said genes with a reference expression level of said genes characteristic of metastatic breast cancer cells;
wherein expression of KIAA1199 and NDRG1 in said isolated breast cancer cell that is equal to or greater than the reference expression level indicates that the isolated breast cancer cell has metastatic capacity; and
c) administering a therapeutically effective amount of a Coco inhibitor or BMP agonist if the patient is at risk for relapse and/or metastasis to the lung.

23. The method of claim 22, further comprising contacting said sample containing a gene expression product from the isolated breast cancer cell with a gene expression panel or array comprising nucleic acids capable of hybridizing to an expression product of a gene selected from the group consisting of ALDH6A1, PTDSS1, SCD, MYO5C, TFPI, PNMA2, TMEM45A, PLAT, HSD17B10, PLAT, EVI2B, and JAK1.

24. The method of claim 22, wherein genes ALDH6A1, PTDSS1, SCD, MYO5C, TFPI, PNMA2, TMEM45A, PLAT, PLAT, EVI2B, and JAK1 are upregulated in metastatic breast cancer and HSD17B10 is downregulated.

25. A method of identifying genes involved in metastasis and outgrowth, the method comprising:
(a) transducing a poorly metastatic cell with a retroviral cDNA library from highly metastatic cells;
(b) introducing transduced cells into a first site of a syngeneic non-human mammal;
(c) recovering cells that have colonized a second site remote from the first site;
(d) rescuing and sequencing integrated provirus recovered from a cell from said second site; and
(e) identifying a cDNA from said provirus that confers a highly metastatic phenotype on poorly metastatic cells when said cDNA is introduced into untransduced cells.

26. The method of claim 25, wherein said non-human mammal is a mouse.

27. The method of claim 25, wherein said first site is mammary fat pad and second site is lung.

28. The method of claim 25, wherein said poorly metastatic cell is a mammary carcinoma cell.

29. A method of treatment for a breast cancer patient, comprising
(a) contacting a sample containing a gene expression product from a breast cancer cell or tissue sample from the patient with at least one nucleic acid capable of hybridizing to genes KIAA1199 and NDRG1 to determine an expression level of KIAA1199 and NDRG1 in the sample from said patient;
(b) comparing the expression level in said sample from the patient with a reference expression level of said genes in a cell characteristic of metastatic breast cancer,
(c) treating the patient aggressively when expression of KIAA1199 and NDRG1 in said isolated breast cancer cell is equal to or greater than the reference expression level of said genes in a cell characteristic of metastatic breast cancer cells.
